SEQUENCE LISTING (39 sequences)

SEQ ID NO: 1 - protein, 11 aa, Mus musculus
GASENIYGALN

SEQ ID NO: 2 - protein, 7 aa, Mus musculus
GASNLAD

SEQ ID NO: 3 - protein, 9 aa, Mus musculus
QNVLSSPYT

SEQ ID NO: 4 - protein, 11 aa, Homo sapiens
QASENIYGALN

SEQ ID NO: 5 - protein, 7 aa, Homo sapiens
GASNLAT

SEQ ID NO: 6 - protein, 9 aa, Homo sapiens
QQVLSSPYT

SEQ ID NO: 7 - protein, 10 aa, Mus musculus
GYTFTDYAMH

SEQ ID NO: 8 - protein, 17 aa, Mus musculus
LISTYSGDTKYNQNFKG

SEQ ID NO: 9 - protein, 12 aa, Mus musculus
GDYSGSRYWFAY

SEQ ID NO: 10 - protein, 17 aa, Homo sapiens
LISTYSGDTKYNQNFQG

SEQ ID NO: 11 - protein, 12 aa, Homo sapiens
GDYSGSRYWFAY

SEQ ID NO: 12 - DNA, 965 bp, Mus musculus
  source 1..965: mol_type = unassigned DNA
gcggggcagcctcacacagaacacacacagatatgggtgtacccactcagctcctgttgctgtggcttacagtcgtagttgtcagatgtgacatccagatgactcagtctccagcttcactgtctgcatctgtgggagaaactgtcaccatcacatgtggagcaagtgagaatatttacggtgctttaaattggtatcagcggaaacagggaaaatctcctcagctcctgatttatggcgcaagtaatttggcagatggcatgtcatcgaggttcagtggcagtggatctggtagacagtattctctcaagatcagtagcctgcatcctgacgatgttgcaacgtattactgtcaaaatgtattaagtagtccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacaccaacttcacccattgtcaagagcttcaacaggaatgagtgttagagacaaaggtcctgagacgccaccaccagctccccagctccatcctatcttcccttctaaggtcttggaggcttccccacaagcgacctaccactgttgcggtgctccaaacctcctccccacctccttctcctcctcctccctttccttggcttttatcatgctaatatttgcagaaaatattcaataaagtgagtctttgcacaaaaaaaaaaaaaaaaaaaaaaaaaa

SEQ ID NO: 13 - DNA, 1575 bp, Mus musculus
  source 1..1575: mol_type = unassigned DNA
acgcgggacacagtagtctctacagtcacaggagtacacaggacattgccatgggttggagctgtatcatcttctttctggtagcaacagctacaggtgtgcactcccaggtccagctgcagcagtctgggcctgaggtggtgaggcctggggtctcagtgaagatttcctgcaagggttccggctacacattcactgattatgctatgcactgggtgaagcagagtcatgcaaagagtctcgagtggattggacttattagtacttacagtggtgatacaaagtacaaccagaactttaagggcaaggccacaatgactgtagacaaatcctccaacacagcctatatggaacttgccagattgacatctgaggattctgccatctattactgtgcaagaggggattattccggtagtaggtactggtttgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatcccagtgtccttggagccctctggccctacaggactttgacacctacctccacccctccctgtataaataaagcacccagcactgcctcgggaccctgcataaaaaaaaaaaaaaaaaaaaaaaaaaaa

SEQ ID NO: 14 - protein, 107 aa, Mus musculus
LMTQSPASLSASVGETVTITCGASENIYGALNWYQRKQGKSPQLLIYGASNLADGMSSRFSGSGSGRQYSLKISSLHPDDVATYYCQNVLSSPYTFGGGTKLEIKKG

SEQ ID NO: 15 - protein, 116 aa, Mus musculus
LEESGPEVVRPGVSVKISCKGSGYTFTDYAMHWVKQSHAKSLEWIGLISTYSGDTKYNQNFKGKATMTVDKSSNTAYMELARLTSEDSAIYYCARGDYSGSRYWFAYWGQGTTVTR

SEQ ID NO: 16 - protein, 107 aa, Mus musculus
DIQMTQSPASLSASVGETVTITCGASENIYGALNWYQRKQGKSPQLLIYGASNLADGMSSRFSGSGSGRQYSLKISSLHPDDVATYYCQNVLSSPYTFGGGTKLEIK

SEQ ID NO: 17 - protein, 107 aa, synthetic construct
  REGION 1..107: note = Humanized antibody sequence
DIQMTQSPSSLSASVGDRVTITCGASENIYGALNWYQRKPGKSPKLLIYGASNLADGMPSRFSGSGSGRQYTLTISSLQPEDVATYYCQNVLSSPYTFGGGTKLEIK

SEQ ID NO: 18 - protein, 107 aa, synthetic construct; identical to SEQ ID NO: 17
  REGION 1..107: note = Humanized antibody sequence

SEQ ID NO: 19 - protein, 107 aa, synthetic construct
  REGION 1..107: note = Humanized antibody sequence
DIQMTQSPSSLSASVGDRVTITCQASENIYGALNWYQRKPGKSPKLLIYGASNLATGMPSRFSGSGSGTDYTFTISSLQPEDIATYYCQNVLSSPYTFGGGTKLEIK

SEQ ID NO: 20 - protein, 107 aa, synthetic construct
  REGION 1..107: note = Humanized antibody sequence
DIQMTQSPSSLSASVGDRVTITCQASENIYGALNWYQRKPGKSPKLLIYGASNLATGMPSRFSGSGSGRQYTFTISSLQPEDIATYYCQQVLSSPYTFGGGTKLEIK

SEQ ID NO: 21 - protein, 107 aa, synthetic construct
  REGION 1..107: note = Humanized antibody sequence
DIQMTQSPSSLSASVGDRVTITCGASENIYGALNWYQRKPGKSPNLLIYGASNLADGMPSRFSGSGSGRQYTLTISSLQPEDVATYYCQNVLSSPYTFGGGTKLEIK

SEQ ID NO: 22 - protein, 121 aa, Mus musculus
QVQLQQSGPEVVRPGVSVKISCKGSGYTFTDYAMHWVKQSHAKSLEWIGLISTYSGDTKYNQNFKGKATMTVDKSSNTAYMELARLTSEDSAIYYCARGDYSGSRYWFAYWGQGTLVTVSA

SEQ ID NO: 23 - protein, 121 aa, synthetic construct
  REGION 1..121: note = Humanized antibody sequence
QVQLVQSGAEVKKPGASVKVSCKGSGYTFTDYAMHWVRQAPGQRLEWIGLISTYSGDTKYNQNFKGKATMTVDKSASTAYMELSSLRSEDTAVYYCARGDYSGSRYWFAYWGQGTLVTVSS

SEQ ID NO: 24 - protein, 121 aa, synthetic construct
  REGION 1..121: note = Humanized antibody sequence
QVQLVQSGAEVKKPGASVKVSCKGSGYTFTDYAMHWVRQAPGQGLEWIGLISTYSGDTKYNQNFKGKATMTVDTSISTAYMELSRLRSDDTAIYYCARGDYSGSRYWFAYWGQGTLVTVSS

SEQ ID NO: 25 - protein, 121 aa, synthetic construct
  REGION 1..121: note = Humanized antibody sequence
QVQLVQSGAEVKKPGASVKVSCKASGYTFTDYAMHWVRQAPGQRLEWMGLISTYSGDTKYSQKFQGRVTMTVDKSASTAYMELSSLRSEDTAVYYCARGDYSGSRYWFAYWGQGTLVTVSS

SEQ ID NO: 26 - protein, 121 aa, synthetic construct
  REGION 1..121: note = Humanized antibody sequence
QVQLVQSGAEVKKPGASVKVSCKASGYTFTDYAMHWVRQAPGQRLEWIGLISTYSGDTKYNQKFQGKATMTVDKSASTAYMELSSLRSEDTAVYYCARGDYSGSRYWFAYWGQGTLVTVSS

SEQ ID NO: 27 - protein, 121 aa, synthetic construct
  REGION 1..121: note = Humanized antibody sequence
QVQLVQSGAEVKKPGASVKVSCKGSGYTFTDYAMHWVRQVHAQGLEWIGLISTYSGDTKYNQNFKGKATMTVDKSTSTAYMELSSLRSEDTAVYYCARGDYSGSRYWFAYWGQGTLVTVSS

SEQ ID NO: 28 - protein, 470 aa, synthetic construct
  REGION 1..470: note = Humanized antibody sequence (h16C3-Abb* heavy chain)
  REGION 1..19: note = MISC_FEATURE - Leader sequence
  REGION 20..140: note = MISC_FEATURE - Variable region
  REGION 141..470: note = MISC_FEATURE - Constant region
  REGION 45..54: note = MISC_FEATURE - CDR1
  REGION 70..85: note = MISC_FEATURE - CDR2
  REGION 118..129: note = MISC_FEATURE - CDR3
MGWSCIIFFLVATATGVHSQVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 233 aa, synthetic construct
  REGION 1..233: note = Humanized antibody sequence (h16C3-Abb* light chain)
  REGION 1..19: note = MISC_FEATURE - Leader sequence
  REGION 20..127: note = MISC_FEATURE - Variable region
  REGION 128..233: note = MISC_FEATURE - Constant region
  REGION 43..53: note = MISC_FEATURE - CDR1
  REGION 69..75: note = MISC_FEATURE - CDR2
  REGION 108..116: note = MISC_FEATURE - CDR3
MGVPTQLLLLWLTVVVVRCDIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 17 aa, Homo sapiens
LISTYSGDTKYSQKFQG

SEQ ID NO: 31 - protein, 17 aa, synthetic construct
  REGION 1..17: note = Humanized antibody sequence
LISTYSGDTKYNQKFQG

SEQ ID NO: 32 - protein, 10 aa, synthetic construct
  REGION 1..10: note = Humanized antibody sequence (h16C3-Abb* heavy chain CDR1)
GYTFTDYAMH

SEQ ID NO: 33 - protein, 16 aa, synthetic construct
  REGION 1..16: note = Humanized antibody sequence (h16C3-Abb* heavy chain CDR2)
ISTYSGDTKYNQNFQG

SEQ ID NO: 34 - protein, 12 aa, synthetic construct
  REGION 1..12: note = Humanized antibody sequence (h16C3-Abb* heavy chain CDR3)
GDYSGSRYWFAY

SEQ ID NO: 35 - protein, 11 aa, synthetic construct
  REGION 1..11: note = Humanized antibody sequence (h16C3-Abb* light chain CDR1)
QASENIYGALN

SEQ ID NO: 36 - protein, 7 aa, synthetic construct
  REGION 1..7: note = Humanized antibody sequence (h16C3-Abb* light chain CDR2)
GASNLAT

SEQ ID NO: 37 - protein, 9 aa, synthetic construct
  REGION 1..9: note = Humanized antibody sequence (h16C3-Abb* light chain CDR3)
QQVLSSPYT

SEQ ID NO: 38 - protein, 121 aa, synthetic construct
  REGION 1..121: note = Humanized antibody sequence (h16C3-Abb* heavy chain variable)
  REGION 26..35: note = MISC_FEATURE - CDR1
  REGION 51..66: note = MISC_FEATURE - CDR2
  REGION 99..110: note = MISC_FEATURE - CDR3
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSS

SEQ ID NO: 39 - protein, 108 aa, synthetic construct
  REGION 1..108: note = Humanized antibody sequence (h16C3-Abb* light chain)
  REGION 24..34: note = MISC_FEATURE - CDR1
  REGION 50..56: note = MISC_FEATURE - CDR2
  REGION 89..97: note = MISC_FEATURE - CDR3
DIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIKR

(19) United States
(12) Patent Application Publication
(10) Pub. No.: US 2026/0083840 A1
(43) Pub. Date: Mar. 26, 2026

(54) METHODS FOR ABLATING MYELOID DERIVED SUPPRESSOR CELLS USING NEO-201 ANTIBODY

(71) Applicant: PRECISION BIOLOGICS, INC., Bethesda, MD (US)

(72) Inventors: Kwong Y. TSANG, Bethesda, MD (US); Massimo FANTINI, Bethesda, MD (US); Philip M. ARLEN, Bethesda, MD (US)

(21) Appl. No.: 18/867,032

(22) PCT Filed: May 18, 2023
(86) PCT No.: PCT/US2023/067188

§ 371 Date: Nov. 19, 2024

Related U.S. Application Data
(60) Provisional application No. 63/343,559, filed on May 19, 2022.
(60) Provisional application No. 63/494,094, filed on Apr. 4, 2023.

(51) Int. Cl. A61K 39/395; A61P 35/00; C07K 16/28; C07K 16/30; G01N 33/574

(52) U.S. Cl. CPC A61K 39/39558; A61K 39/39541; A61P 35/00; C07K 16/2803; C07K 16/3007; G01N 33/5758; C07K 2317/24; C07K 2317/732; C07K 2317/734; C07K 2317/76

(57) ABSTRACT

NEO-201, an antibody that specifically binds to glycosylated peptides carrying core-1 and/or extended core-1 O-glycans comprised in CEACAM5 and CEAMCAM6 but not to aglycosylated CEACAM5 or aglycosylated CEAMCAM6 surprisingly has been shown to bind to and kill granulocyte myeloid derived suppressor cells (gMDSCs). gMDSCs are known to suppress innate immunity in different cancers and infectious diseases, among other conditions. Based thereon the use of NEO-201 alone or in combination for treating cancers and infectious diseases and other conditions wherein gMDSCs suppress innate immunity against the disease are provided. These methods optionally include detecting gMDSCs before, during or after NEO-201 treatment. Diagnostic methods, therapeutic methods, and combination therapies using NEO-201 optionally in combination with another agent in order to ablate gMDSCs and disease cells are also described.

### CROSS-REFERENCE TO RELATED APPLICATION

This application claims priority to U.S. Provisional Application No. 63/343,559 filed on May 19, 2022, and U.S. Provisional Application No. 63/494,094 filed on Apr. 4, 2023, the contents of both of which are incorporated by reference in their entirety.

### SEQUENCE LISTING INFORMATION

This application includes as part of its disclosure a biological sequence listing in the file named “1143282o005000.txt”, created on May 18, 2022, having a size of 32,427 bytes, which is hereby incorporated by reference in its entirety.

### BACKGROUND

The human carcinoembryonic antigen (CEA) family is a composed of 29 genes tandemly arranged on chromosome 19q13.2. Based on nucleotide homologies, these genes are classified into two major subfamilies, the CEACAM and pregnancy-specific glycoprotein subgroups. The CEACAM-encoded proteins include CEA (CEACAM5), CEA-related cell adhesion molecules (CEACAM1, CEACAM3, CEACAM4, CEACAM6, CEACAM7 and CEACAM8. CEACAM family belongs to the Ig superfamily. Structurally, each of the human CEACAMs contain one N-terminal domain that includes 108-110 amino acid and is homologous to Ig variable domains, followed by a different number (zero to six) of Ig C2-type constant-like domains. The CEACAM proteins can interact homophilically and heterophilically with each other. CEACAM1 is a unique protein within this family because it contains an ITIM (immunoreceptor tyrosine-based inhibitory motif) like PD1 in its cytoplasmic domain. This inhibitory effect is triggered by phosphorylation of tyrosine residues with the ITIM, which results in recruitment of the Src homology 2 domain-containing tyrosine phosphatase-1 and -2. The CEACAM1 protein is expressed on a variety of immune cells including monocytes, granulocytes, activated T cells, B cells and NK cells. CEACAM1 occurs as several isoforms, the two major ones being CEACAM1-L and CEACAM1-S that have long (L), or short (S) cytoplasmic domains, respectively. CEACAM1-S expression is totally lacking in human leukocytes. CEACAM1-L is expressed on subpopulation of activated human NK cells that are negative for CD16 but positive for CD56. Heterophilic interactions between CEA on tumor cells and CEACAM1 on NK cells inhibit NK cell cytotoxicity against tumor cells.

NEO-201 is a humanized IgG1 mAb that binds to cancer proteins carrying core-1 and/or extended core-1 O-glycans, including tumor-associated variants of CEACAM family members, particularly cancer-associated variants of CEACAM5 and CEACAM6 (Zeligs et al., Cancer Res. Jul. 1, 2017 (77) (13 Supplement) 3025) and Tsang K Y, Fantini M, Zaki A, Mavroukakis S A, Morelli M P, Annunziata C M, Arlen P M. “Identification of the O-Glycan Epitope Targeted by the Anti-Human Carcinoma Monoclonal Antibody (mAb) NEO-201”, Cancers (Basel). 2022 Oct. 12; 14(20):4999. doi: 10.3390/cancers14204999. NEO-201 has been demonstrated to be reactive against certain carcinomas, but not reactive against most normal tissues. Applicant's prior U.S. Pat. Nos. 5,688,657, 7,314,622, 7,491,801, 7,763,720, 7,829,678, 8,470,326, 8,524,456, 8,535,667, 8,802,090, 9,034,588, 9,068,014, 9,371,375, 9,592,290, 9,718,866, and RE39,760, disclose the use of NEO-201 for the diagnosis and treatment of colon and pancreas cancers. Additionally, recently in PCT Application No.: ______, Applicant disclosed the use of NEO-201 for the treatment of hematological malignancies which express cancer proteins carrying core-1 and/or extended core-1 O-glycans, including CEACAM5 and/or CEACAM6.

However, to Applicant's knowledge, the use of NEO-201, or any other antibody which targets CEACAM5 and/or CEACAM6 to detect and/or deplete myeloid-derived suppressor cells (MDSCs) has not earlier been reported. As is known in the art MDSCs are a population of myeloid cells generated during a large array of pathologic conditions ranging from cancer, infection to obesity. These cells represent a pathologic state of activation of monocytes and relatively immature neutrophils. MDSCs are characterized by a distinct set of genomic and biochemical features, and can be distinguished from granulocytes and other cells by their expression of specific surface molecules. The salient feature of these cells is their ability to inhibit T cell function and thus contribute to the pathogenesis of various diseases. Particularly these cells are known to contribute to the pathology of diseases including cancer, infectious diseases, autoimmunity, obesity and pregnancy.

Accordingly, methods for detecting and/or ablating MDSCs have great therapeutic potential.

### BRIEF DESCRIPTION AND EXEMPLARY EMBODIMENTS

We have previously shown NEO-201 to bind to cancer-associated variants of CEACAM5 and CEACAM6, specifically via a cancer-associated glycosylation variant of these proteins carrying core-1 and/or extended core-1 O-glycans. NEO-201 is a humanized IgG1 monoclonal antibody that was derived from an immunogenic preparation of tumor-associated antigens from pooled allogeneic colon tumor tissue extracts. NEO-201 is reactive against a majority of tumor tissues from many different carcinomas, but is not reactive to the majority of the normal tissues. Functional analysis revealed that NEO-201 is capable of mediating both antibody-dependent cellular cytotoxicity (ADCC) and complement-dependent cytotoxicity (CDC) against tumor cells. Previous studies have demonstrated that NEO-201 attenuates the grown of human tumor xenografts in mice, and demonstrates safety and tolerability in non-human primates with a transient decrease in circulating neutrophils being the only adverse effect observed.

Applicants show herein that NEO-201 binds to granulocytes and further binds to granulocyte derived MDSCs (gMDSCs) and can kill gMDSCs via ADCC. As noted previously, MDSCs are a population of myeloid cells generated during a large array of pathologic conditions ranging from cancer to obesity which inter alia inhibit T cell function and contribute to the pathogenesis of various diseases. Particularly these cells contribute to the pathology of diseases including cancer, infectious diseases, autoimmunity, obesity and pregnancy.

MDSCs have emerged as a universal regulator of immune function in many pathologic conditions. MDSCs consist of two large groups of cells: granulocytic or polymorphonuclear (PMN-MDSC or gMDSC) and monocytic (M-MDSC). PMN-MDSCs or gMDSCs are phenotypically and morphologically similar to neutrophils, whereas M-MDSCs are more similar to monocytes (Gabrilovich D I et al., “Coordinated regulation of myeloid cells by tumors”, Nat Rev Immunol. 2012; 12(4):253-268). Also the existence of a third small population of MDSCs that are represented by cells with colony forming activity and other myeloid precursors has been reported which are referred to as early-stage MDSC (eMDSC) (Dumitru C A et al., “Neutrophils and granulocytic myeloid-derived suppressor cells: immunophenotyping, cell biology and clinical relevance in human oncology”, Cancer Immunol Immunother. 2012; 61(8):1155-11673).

Accordingly, based on its demonstrated capability to detect and/or ablate gMDSCs, NEO-201 potentially may be used in treating and/or monitoring the disease status of any condition where gMDSCs are involved in the disease pathology, most particularly cancer, and particularly may be useful in treating cancers that do not express CEACAM5 and/or CEACAM6, and for treating chronic infectious conditions where gMDSCs are known to suppress innate immunity. Moreover, NEO-201 especially should be useful in combination therapies, e.g., in combination with other therapeutics, e.g., other drugs or treatments which ablate and/or or inhibit the activity of MDSCs, as well as other therapeutic antibodies, checkpoint inhibitors, chemotherapeutics, and the like since NEO-201, because of its ability to deplete gMDSCs, should potentiate the efficacy of other therapeutics, e.g., by potentiating innate immunity, e.g., innate anti-tumor or anti-infectious agent responses, even in subjects who previously were resistant to treatment.

Based on the foregoing, in one embodiment the invention provides a method of killing or ablating granulocyte derived myeloid derived suppressor cells (gMDSCs) in a patient in need thereof, comprising administering to the patient an effective amount of an antibody or antibody fragment which binds to glycosylated CEACAM 5 and CEACAM6 carrying core-1 and/or extended core-1 O-glycans but not to aglycosylated CEACAM 5 or aglycosylated CEACAM6, optionally wherein said antibody or antibody fragment recognizes an O-glycosylated epitope binding to the threonine in the region of amino acids from 310 to 318 (RTTVTTITV) of CEACAM5 and to the Threonine and Serine in the region of amino acids 312 to 320 (TVTMITVSG) of CEACAM6.

In another embodiment the invention provides a method of killing or ablating granulocyte myeloid derived suppressor cells (gMDSCs) in a patient in need thereof, comprising administering to the patient an effective amount of NEO-201 or an antigen binding fragment thereof.

In another embodiment the invention provides a method of reversing tolerance and/or restoring innate immunity, e.g., innate antitumor immunity or innate anti-infectious immunity in a patient in need thereof by killing or ablating granulocyte myeloid derived suppressor cells (gMDSCs) in the patient, comprising administering to the patient an effective amount of NEO-201 or an antigen binding fragment thereof.

In another embodiment the invention provides a method of reversing resistance or tolerance to an anti-cancer or anti-infectious agent treatment, e.g., an immune modulatory antibody, a checkpoint inhibitor antibody or fusion protein, or a chemotherapeutic agent, which resistance or tolerance involves granulocyte myeloid derived suppressor cells, by administering NEO-201 alone or in combination with another treatment, e.g., an immune modulatory antibody, a checkpoint inhibitor antibody or fusion protein, or a chemotherapeutic agent in order to reverse such resistance or tolerance.

In another embodiment the invention provides a method of treating or preventing cancer or infection reoccurrence by administering NEO-201 alone or in combination with another treatment, e.g., an immune modulatory antibody, a checkpoint inhibitor antibody or fusion protein, or a chemotherapeutic agent in order to suppress the proliferation of MDSCs, thereby reestablishing innate immunity.

In exemplary embodiments, in any one of the previous methods, the gMDSCs are expressing O-glycans selected from one or more of O1, O2, O6, O23, O26 and O39 O-glycans having the structure shown in the array in FIG. 2 and/or in FIG. 5.

In exemplary embodiments, in any one of the previous methods, the gMDSCs are derived from neutrophils expressing O6, O1 or O2 O-glycans having a structure shown in the array in FIG. 2 and/or in FIG. 5.

In exemplary embodiments, in any one of the previous methods, the gMDSCs are derived from neutrophils expressing express O6 O-glycans as shown in the array in FIG. 2 and/or in FIG. 5.

In exemplary embodiments, in any one of the previous methods, the gMDSCs can express Tn antigens or Core 1, 2, 4 or 4 O-glycans having the structures shown in FIG. 1.

In exemplary embodiments, in any one of the previous methods, the patient has a cancer or infectious disease wherein the disease pathology and/or immunosuppression of innate immunity against the disease involves gMDSCs.

In exemplary embodiments, in any one of the previous methods, the antibody or antigen binding fragment is directly or indirectly linked to a cytotoxic agent, optionally a radionuclide or chemotherapeutic.

In exemplary embodiments, in any one of the previous methods, the antibody or antigen binding fragment is directly or indirectly linked to a label, optionally a fluorescent or radioactive label.

In exemplary embodiments, in any one of the previous methods, the treated subject has a cancer where MDSCs are involved in disease pathology, optionally wherein the treated cancer cells do not express or overexpress an antigen bound by NEO-201.

In exemplary embodiments, in any one of the previous methods, the treated subject has a cancer where MDSCs are involved in disease pathology, optionally Adrenal Cancer, Anal Cancer, Bile Duct Cancer, Bladder Cancer, Bone Cancer, Brain/CNS Tumors In Adults, Brain/CNS Tumors In Children, Breast Cancer, Breast Cancer In Men, Cancer in Adolescents, Cancer in Children, Cancer in Young Adults, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Leukemia—Acute Lymphocytic (ALL) in Adults, Leukemia—Acute Myeloid (AML), Leukemia—Chronic Lymphocytic (CLL), Leukemia—Chronic Myeloid (CML), Leukemia—Chronic Myelomonocytic (CMML), Leukemia in Children, Liver Cancer, Lung Cancer, Lung Cancer—Non-Small Cell, Lung Cancer—Small Cell, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Skin Cancer—Basal and Squamous Cell, Skin Cancer—Melanoma, Skin Cancer—Merkel Cell, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor, optionally wherein the treated cancer cells do not express or overexpress an antigen bound by NEO-201.

In exemplary embodiments, in any one of the previous methods, the treated subject has a cancer where MDSCs are involved in disease pathology, optionally a cancer and/or a tumor selected from the group consisting of lung cancer, breast cancer, triple negative breast cancer (TNBC), colorectal cancer, liver cancer, stomach cancer, colon cancer, non-small cell lung cancer (NSCLC), bone cancer, pancreatic cancer, skin cancer, head or neck cancer, cutaneous or intraocular melanoma, uterine cancer, ovarian cancer, colorectal cancer, small intestine cancer, rectal cancer, anal cancer, fallopian tube cancer, endometrial cancer, cervical cancer, vaginal cancer, vulva cancer, Hodgkin's disease, esophageal cancer, small intestine cancer, lymph node cancer, bladdercancer, gallbladder cancer, endocrine cancer, thyroid cancer, parathyroid cancer, adrenal cancer, soft tissue sarcoma, urethra cancer, penis cancer, prostate cancer, adenocarcinoma, chronic or acute leukemia, lymphocytic lymphoma, bladder cancer, kidney or ureter cancer, renal cell carcinoma, renal pelvic carcinoma, central nervous system tumor, primary CNS tumor, spinal cord tumor, brainstem glioma, and pituitary adenoma, optionally wherein the treated cancer cells do not express or overexpress an antigen bound by NEO-201.

In exemplary embodiments, in any one of the previous methods, the treated cancer or infection is not characterized by the expression of glycosylated CEACAM 5 and/or glycosylated CEACAM6; and/or is not characterized by the increased expression of glycosylated CEACAM 5 and/or glycosylated CEACAM6.

In exemplary embodiments, in any one of the previous methods, the treated subject has a cancer where MDSCs are involved in disease pathology, and treatment elicits one or more of (i) increased T-cell response, (ii) increased antigen presentation, (iii) reduced proliferation of MDSCs and/or (iv) reduced Treg recruitment.

In exemplary embodiments, in any one of the previous methods, the treated subject has stage I, stage II, stage III, or stage IV cancer involving MDSCs.

In exemplary embodiments, in any one of the previous methods, the antibody or fragment, optionally NEO-201, reduces, eliminates or slows or arrests the growth of tumors wherein in a patient wherein antitumor immunity was previous suppressed by gMDSCs, reduces tumor burden in the individual, inhibits tumor growth, and/or increases survival of the individual.

In exemplary embodiments, in any one of the previous methods, the subject has an infectious condition wherein the disease pathology involves MDSCs.

In exemplary embodiments, in any one of the previous methods, the subject has a bacterial infection involving MDSCs, optionally Bacillus anthraces, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis and Enterococcus faecium, Escherichia coli (generally), Enterotoxigenic Escherichia coli (ETEC), Enteropathogenic E. coli, E. coli O157:H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia, Salmonella typhi, Salmonella typhimurium, Shigella sonnei, and/or Staphylococcus aureus infection.

In exemplary embodiments, in any one of the previous methods, the subject has a chronic or acute viral infection involving MDSCs, optionally associated with Respiratory Viruses, such as, Adenoviruses, Avian influenza, Influenza virus type A, Influenza virus type B, Measles, Parainfluenza virus, Respiratory syncytial virus (RSV), Rhinoviruses, SARS-CoV, Gastro-enteric Viruses, such as, Coxsackie viruses, Enteroviruses, Poliovirus, Rotavirus, Hepatitis Viruses, such as, Hepatitis B virus, Hepatitis C virus, Bovine viral diarrhea virus (surrogate), Herpes Viruses, such as, Herpes simplex 1, Herpes simplex 2, Human cytomegalovirus, Varicella zoster virus, Retroviruses, such as, Human immunodeficiency virus 1 (HIV-1), Human immunodeficiency virus 2 (HIV-2), Simian immunodeficiency virus (SIV), Simian human immunodeficiency virus (SHIV), Viral Select Agents/Emerging Viral Pathogens, such as, Avian influenza, Dengue virus, Hantavirus, Hemorrhagic fever viruses, Lymphocytic choriomeningitis virus, Smallpox virus surrogates, Cowpox, Monkeypox, Rabbitpox, Vaccinia virus, Venezuelan equine encephalomyelitis virus (VEE), West Nile virus, Yellow fever virus.

In exemplary embodiments, in any one of the previous methods, the subject has a condition involving MDSCs wherein MDSCs are involved in suppressing innate immunity, optionally acquired immune deficiency syndrome (AIDS), acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, anti-synthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behcet's disease, Berger's disease, Bickerstaff's encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barre syndrome (GBS), Hashimoto's encephalopathy, Hashimoto's thyroiditis, heart disease, Henoch-Schönlein purpura, herpes gestationis (aka gestational pemphigoid), hidradenitis suppurativa, HIV infection, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenic purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosis, linear IgA disease (LAD), lupoid hepatitis (aka autoimmune hepatitis), lupus erythematosus, lymphomatoid granulomatosis, Majeed syndrome, malignancies including cancers (e.g., sarcoma, Kaposi's sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, ocular cicatricial pemphigoid, opsoclonus myoclonus syndrome, Ord's thyroiditis, palindromic rheumatism, PANDAS (pediatric autoimmune neuropsychiatric disorders associated with Streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, sarcoidosis, schizophrenia, Schmidt syndrome, Schnitzler syndrome, scleritis, scleroderma, sepsis, serum Sickness, Sjogren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome,) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

In exemplary embodiments, in any one of the previous methods, the gMDSCs in the patient may be detected and monitored prior, during and after treatment has been completed and/or after the patient has gone into remission.

In exemplary embodiments, in any one of the previous methods, the gMDSCs in the patient may be detected prior to treatment in determining whether the patient will potentially benefit from NEO-201 treatment.

In exemplary embodiments, in any one of the previous methods, the gMDSCs may be detected in a biological sample using one or more ligands, e.g., antibodies that recognize specific biomarkers expressed on gMDSCs, optionally LOX-1, CD11b, CD15, CD66b and glycosylated CEACAM5 and CEACAM6 antigens expressing core-1 and/or extended core-1 O glycans recognized by NEO-201.

In exemplary embodiments, in any one of the previous methods, the number or concentration of gMDSCs in a sample of a subject with a cancer involving gMDSCs, wherein the patient is being treated for such cancer with NEO-201 alone or in combination with another therapeutic agent may be used to monitor the progression of the cancer (with or without treatment).

In exemplary embodiments, in any one of the previous methods, the number or concentration of gMDSCs in a sample of a subject with a cancer involving gMDSCs, wherein the patient is being treated for such cancer with NEO-201 alone or in combination with another therapeutic agent may be used to determine whether NEO-201 may be beneficial in treating the cancer, alone or in combination with another therapeutic agent.

In exemplary embodiments, in any one of the previous methods, the number or concentration of gMDSCs in a sample of a subject with a cancer involving gMDSCs, may be used to develop a dosing regimen of NEO-201 alone or in combination with another therapeutic agent.

In exemplary embodiments, in any one of the previous methods, the level of gMDSCs in a patient sample, such as a blood or biopsy sample, may be used to determine cancer prognosis prior, during or after NEO-201 treatment, which method optionally comprises contacting said gMDSCs with a NEO-201 antibody.

In exemplary embodiments, in any one of the previous methods, said detecting comprises cell sorting, optionally fluorescence activated cell sorting, thereby producing a sample enriched for and/or depleted of cells positive for NEO-201 antigen expression, e.g., gMDSCs.

In exemplary embodiments, any one of the previous methods may include detecting and/or staining gMDSCs by contacting cells with a NEO-201 antibody and detecting cells that express NEO-201 wherein optionally NEO-201 is directly or indirectly labeled.

In exemplary embodiments, in any one of the previous methods, gMDSCs may be isolated by contacting a patient sample with a support comprising a NEO-201 antibody and/or using other antibodies or ligands which recognize other MDSC biomarkers, whereby said MDSCs are retained on said support.

In exemplary embodiments, in any one of the previous methods, the level of MDSCs in a patient sample, such as a blood or biopsy sample, may be used to determine whether a patient has or likely to develop MDSC-mediated immunosuppression.

In exemplary embodiments, in any one of the previous methods, the method may include the administration of another therapeutic agent.

In exemplary embodiments, in any one of the previous methods, the method may include the administration of at least one other therapeutic agent, wherein the administration of NEO-201 or other antibody which binds to glycosylated CEACAM 5 and CEACAM6 carrying core-1 or extended core-1 O glycans, but not to aglycosylated CEACAM5 or aglycosylated CEACAM6 with the at least one other therapeutic agent potentiates the efficacy of the at least one other therapeutic agent.

In exemplary embodiments, in any one of the previous methods, the other therapeutic comprises another therapeutic antibody, checkpoint inhibitor, chemotherapeutic and/or comprises immune cells, optionally CAR-T or CAR-NK cells.

In exemplary embodiments, in any one of the previous methods, the other therapeutic may comprise another moiety which (i) ablates MDSCs, (ii) a moiety which promotes the differentiation of MDSCs, (iii) a moiety which inhibits the migration of MDSCs, (iv) an epigenetic therapy which targets MDSCs, moiety, or (v) a chemotherapeutic which targets MDSCs or a combination of one or more of the foregoing.

In exemplary embodiments, in any one of the previous methods, NEO-201, because of its ability to deplete gMDSCs may potentiate the efficacy of the other therapeutic by potentiating innate immunity, e.g., innate anti-tumor or anti-infectious agent responses, optionally in a subject previously resistant to treatment with the other therapeutic.

In exemplary embodiments, in any one of the previous methods, the method may include another additional therapeutic agent(s) including, without limitation, peptides, nucleic acid molecules, small molecule compounds, antibodies and derivatives thereof.

In exemplary embodiments, in any one of the previous methods, the method may include another therapeutic agent(s), optionally immune checkpoint inhibitors, optionally an anti-PD-1 antibody, an anti-PD-L1 antibody, an anti-CTLA-4 antibody, an anti-CD28 antibody, an anti-TIGIT antibody, an anti-LAGS antibody, an anti-TIM3 antibody, an anti-GITR antibody, an anti-4-1BB antibody, or an anti-OX-40 antibody and/or said additional therapeutic agent(s) include one that targets adenosine A2A receptor (AZAR), B7-H3 (also known as CD276); B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

In exemplary embodiments, in any one of the previous methods, the method may include another therapeutic agent(s), e.g., a CSF-1/1R binding agent or inhibitor, or (a) microtubule inhibitors, topoisomerase inhibitors, platins, alkylating agents, and anti-metabolites; (b) MK-2206, ON 013105, RTA 402, BI 2536, Sorafenib, ISIS-STAT3Rx, a microtubule inhibitor, a topoisomerase inhibitor, a platin, an alkylating agent, an anti-metabolite, paclitaxel, gemcitabine, doxorubicin, vinblastine, etoposide, 5-fluorouracil, carboplatin, altretamine, aminoglutethimide, amsacrine, anastrozole, azacytidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and/or vinorelbine; (c) 1-D-ribofuranosyl-1,2,4-triazole-3 carboxamide, 9->2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir; (d) a PD-1 inhibitor or anti-PD-1 antibody such as KEYTRUDA® (pembrolizumab), OPDIVO® (nivolumab), or LIBTAYO (cemiplimab); (e) a PD-L1 inhibitor or anti-PD-L1 antibody such as TECENTRIQ (atezolizumab), IMFINZI (durvalumab), or BAVENCIO (avelumab); or (f) a CTLA-4 inhibitor or anti-CTLA-4 antibody such as YERVOY® ipilimumab.

In exemplary embodiments, in any one of the previous methods, the patient optionally has been determined to be resistant to treatment with one or more actives because of gMDSC-mediated immunosuppression prior to NEO-201 treatment.

In exemplary embodiments, in any one of the previous methods, the patient optionally has been determined to be resistant to treatment with a therapeutic antibody, optionally one that targets a checkpoint inhibitor prior to treatment with NEO-201.

In exemplary embodiments, in any one of the previous methods, the patient optionally has been determined to be resistant to treatment with a PD-1 or CTLA-4 antagonist, optionally an antibody or fusion protein prior to treatment with NEO-201.

In exemplary embodiments, in any one of the previous methods, the patient optionally has developed a resistance and/or no longer responds to treatment said other therapeutic agent prior to treatment with NEO-201.

In exemplary embodiments, in any one of the previous methods, the patient after treatment with NEO-201 optionally more effectively clinically responds to the other active, optionally another therapeutic antibody or fusion protein, further optionally one that targets a checkpoint inhibitor and/or immune cells, optionally CAR-T or CAR-NK cells.

In exemplary embodiments, in any one of the previous methods, the patient after treatment with NEO-201 optionally more effectively clinically responds to the other active, optionally another therapeutic antibody or fusion protein, further optionally a PD-1 antagonist antibody such as pembrolizumab. nivolumab, cemiplimab, atezolizumab, Atezolizumab, Dostarlimab, durvalumab, lambrolizumab, or avelumab.

In exemplary embodiments, in any one of the previous methods, the patient after treatment with NEO-201 optionally more effectively clinically responds to the other active, optionally another therapeutic antibody or fusion protein, which targets CTLA-4, optionally Yervoy or tremelimumab and/or immune cells, optionally CAR-T or CAR-NK cells.

In exemplary embodiments, in any one of the previous methods, said NEO-201 antibody may comprise the VH and VL CDR sequences contained in SEQ ID NO: 28 and SEQ ID NO: 29.

In exemplary embodiments, in any one of the previous methods, the method may include said NEO-201 antibody may comprise a variable heavy chain sequence having at least 90% identity to SEQ ID NO: 38.

In exemplary embodiments, in any one of the previous methods, said NEO-201 antibody may comprise a variable light chain sequence having at least 90% identity to SEQ ID NO: 39.

In exemplary embodiments, in any one of the previous methods, said NEO-201 antibody may comprise a variable heavy chain sequence having at least 90% identity to SEQ ID NO: 38 and a variable light chain sequence having at least 90% identity to SEQ ID NO: 39.

In exemplary embodiments, in any one of the previous methods, said NEO-201 antibody may comprise a heavy chain sequence having at least 90% identity to amino acids 20-470 of SEQ ID NO: 28 and a light chain sequence having at least 90% identity to amino acids 20-233 of SEQ ID NO: 29.

In exemplary embodiments, in any one of the previous methods, said NEO-201 antibody may comprise or consist of the heavy chain sequence of amino acids 20-470 of SEQ ID NO: 28 and the light chain sequence of amino acids 20-233 of SEQ ID NO: 29.

In exemplary embodiments, in any one of the previous methods, said NEO-201 antibody may comprise a human IgG1 constant domain.

In exemplary embodiments, in any one of the previous methods the antibody preferably comprises the NEO-201 antibody or a variant thereof, e.g., comprising the same CDRs or variable regions as the NEO-201 antibody.

In exemplary embodiments, in any one of the previous methods, said NEO-201 antibody may be conjugated to another moiety.

In exemplary embodiments, in any one of the previous methods, said NEO-201 antibody may be conjugated to another cytotoxic moiety, label, radioactive moiety, or affinity tag.

In exemplary embodiments, in any one of the previous methods, said NEO-201 antibody, preferably a NEO-201 antibody, is comprised in a chimeric antigen receptor (CAR) which is administered to the treated subject.

In exemplary embodiments, in any one of the previous methods, said NEO-201 antibody may be a multispecific or bispecific antibody which targets at least one other antigen, optionally another tumor antigen or an antigen expressed on an immune cell.

In exemplary embodiments, in any one of the previous methods, said other antigen is a checkpoint inhibitor or cytokine or hormone or growth factor.

In exemplary embodiments, in any one of the previous methods, said NEO-201 antibody may be is administered as an immune cell, optionally a human T or NK cell, which immune cell expresses a CAR comprising said antibody.

In other exemplary embodiments the invention provides a method of killing gMDSCs in vivo, comprising administering an effective amount of a NEO-201 antibody to a patient, optionally wherein the patient is being treated with CAR-T or CAR-NK cells.

In other exemplary embodiments the invention provides a method of treating or preventing or reversing gMDSC mediated immunosuppression, comprising administering an effective amount of a NEO-201 antibody to a patient.

In other exemplary embodiments the invention provides a method of potentiating the efficacy of CAR-T or CAR-NK therapy by administering NEO-201 in combination therewith, wherein the CAR may target any of the antigens disclosed herein.

In other exemplary embodiments, any one of the foregoing methods may further comprise administering another therapeutic agent to said patient, optionally wherein said other agent is selected from (a) microtubule inhibitors, topoisomerase inhibitors, platins, alkylating agents, and anti-metabolites; (b) MK-2206, ON 013105, RTA 402, BI 2536, Sorafenib, ISIS-STAT3Rx, a microtubule inhibitor, a topoisomerase inhibitor, a platin, an alkylating agent, an anti-metabolite, paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and/or vinorelbine; (c) 1-D-ribofuranosyl-1,2,4-triazole-3 carboxamide, 9->2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir; (d) a PD-1 inhibitor or anti-PD-1 antibody such as KEYTRUDA® (pembrolizumab), OPDIVO© (nivolumab), or LIBTAYO (cemiplimab); (e) a PD-L1 inhibitor or anti-PD-L1 antibody such as TECENTRIQ (atezolizumab), IMFINZI (durvalumab), or BAVENCIO (avelumab); or (f) a CTLA-4 inhibitor or anti-CTLA-4 antibody such as YERVOY® ipilimumab, or optionally said other agent comprises CAR-T or CAR-NK cells and/or the method further comprises administering an anti-cancer vaccine or CAR-T or CAR-NK cells to said patient.

In other exemplary embodiments the invention provides a method of killing gMDSCs in vitro, comprising contacting a tissue, organ or cell sample suspected of comprising gMDSCs with a NEO-201 antibody, optionally wherein the tissue, organ or cell sample is obtained from a patient with a cancer or infectious disease condition, or wherein the tissue, organ or cell sample is a bone marrow sample from an autologous or allogeneic donor, further optionally further comprising contacting said gMDSCs with complement, further optionally wherein said gMDSCs are killed by ADCC or CDC.

In other exemplary embodiments the invention provides a method of killing gMDSCs in vitro, comprising contacting a tissue, organ or cell sample suspected of comprising gMDSCs with a NEO-201 antibody, optionally wherein the tissue, organ or cell sample is obtained from a patient with a cancer or infectious disease condition, which method further comprises contacting said gMDSCs with effector cells, optionally wherein said effector cells comprise natural killer cells, further optionally wherein said gMDSCs are killed by ADCC.

In other exemplary embodiments, in any one of the foregoing methods said NEO-201 antibody is coupled to a cytotoxic moiety.

In other exemplary embodiments, the invention provides a method of detecting gMDSCs, comprising detecting the expression of the NEO-201 antigen by said gMDSCs and optionally one or more other gMDSC biomarkers, optionally wherein the level of gMDSCs in a patient sample, such as a blood or biopsy sample, is used to determine cancer prognosis or a treatment regimen, which method optionally comprises contacting said gMDSCs with a NEO-201 antibody, wherein optionally said NEO-201 antibody is directly or indirectly coupled to a label, and/or optionally said detecting comprises cell sorting, optionally fluorescence activated cell sorting.

In other exemplary embodiments, the invention provides a method of staining gMDSCs, comprising contacting cells with a NEO-201 antibody, wherein optionally said NEO-201 antibody is directly or indirectly coupled to a label.

In other exemplary embodiments, the invention provides a method of isolating gMDSCs, comprising isolating cells that express the NEO-201 antigen and optionally at least one other gMDSC biomarker, which method optionally includes contacting a sample containing gMDSCs with a NEO-201 antibody, optionally wherein said NEO-201 antibody is directly or indirectly labeled, further optionally wherein said sample is or comprises blood or bone marrow or a tumor biopsy sample, yet further optionally the method includes separating NEO-201 positive cells from NEO-201 negative cells, optionally wherein said gMDSCs are isolated by cell sorting, optionally fluorescence activated cell sorting and/or optionally, wherein said gMDSCs are isolated by contacting sample with a support comprising a NEO-201 antibody, whereby said gMDSCs are retained on said support.

In other exemplary embodiments, in any one of the prior methods, said NEO-201 antibody comprises the CDR sequences contained in SEQ ID NO: 28 and SEQ ID NO: 29, and/or said NEO-201 antibody comprises a variable heavy chain sequence having at least 90% identity to SEQ ID NO: 38, and/or said NEO-201 antibody comprises a variable light chain sequence having at least 90% identity to SEQ ID NO: 39, and/or said NEO-201 antibody comprises a variable heavy chain sequence having at least 90% identity to SEQ ID NO: 38 and a variable light chain sequence having at least 90% identity to SEQ ID NO: 39, and/or said NEO-201 antibody comprises a heavy chain sequence having at least 90% identity to amino acids 20-470 of SEQ ID NO: 28 and a light chain sequence having at least 90% identity to amino acids 20-233 of SEQ ID NO: 29, and/or said NEO-201 antibody comprises all six of the CDR sequences contained in SEQ ID NO: 28 and SEQ ID NO: 29, and/or said NEO-201 antibody comprises a human IgG1 constant domain, and/or said NEO-201 antibody is humanized, and/or said NEO-201 antibody is conjugated to another moiety, and/or said NEO-201 antibody is conjugated to another cytotoxic moiety, label, radioactive moiety, or affinity tag.

### BRIEF DESCRIPTION OF THE SEVERAL VIEWS OF THE DRAWINGS

FIG. 1 contains the structures of O-glycan Cores found in mucins which includes O-glycans which are recognized by the NEO-201 antibody. In the table therein Gal is galactose; GalNAc is N-acetylgalactosamine; GlcNAc is N-acetylglucosamine; Sial is sialic acid; Ser is serine; and Thr is threonine.

FIG. 2 contains an array of different O-glycan structures including O-glycans which are recognized by the NEO-201 antibody.

FIG. 3 contains the amino acid sequence of CEACAM6.

FIG. 4 contains the amino acid sequence of CEACAM5.

FIG. 5 contains the structure of O-glycans such as O1, O2 O6, O23 O26 and O39 O-glycans which are recognized by the NEO-201 antibody.

FIG. 6 to 9 contain the results of flow cytometry analysis of gMDSCs generated from GM-CSF and IL-6 treated neutrophils from 4 normal donors.

FIG. 10 contains a Table showing that 47.59 to 52.58% neutrophils treated with 10 ng/ml of human GM-CSF and 10 ng/ml of human IL-6 were HLA-DR negative and CD33 positive. 76.4% to 88.09% of the HLA-DR negative and CD33 positive population were CD15 positive and CD14 negative. 66.44% to 99.71% of the HLA-DR negative/CD33 positive/CD15 positive/CD14negative population were CD66 positive and NEO-201 positive.

FIG. 11 contains the results of ADCC experiments which demonstrate that when gMDSCs were incubated with PBMCs (E:T 100:1) plus NEO-201 a reduction of 33.01% (18.29% vs 27.23%), and 29.5% (25.95% vs 36.83%) of CD33^{pos}/HLA-DR^{neg} viable cells was observed compared to gMDSCs incubated with PBMCs alone (E:T 100:1) in healthy donor 1 and 2, respectively. A similar reduction of CD33^{pos}/HLA-DR^{reg} viable cells was observed comparing gMDSCs incubated with PBMCs (E:T 50:1) plus NEO-201 with gMDSCs incubated with PBMCs alone (E:T 50:1) in both healthy donors.

FIG. 12 shows a comparison of the percentage of circulating gMDSCs (HLA-DR-/CD33+/CD15+/CD14−/CD66b+ cells) between 2 cancer patients with stable (SD) and 2 cancer patients with progressive disease (PD) at different time points by flow cytometry analysis. In these experiments gMDSCs were gated from alive PBMCs. Data are presented as median of percentage of viable cells expressing gMDSCs markers. Positivity was determined by using fluorescence-minus-one controls. In the figure “HNSCC” refers to “Head and Neck Squamous Cell Carcinoma”.

### DETAILED DESCRIPTION

This disclosure provides a method of depleting or ablating gMDSCs in a patient in need thereof, comprising administering an effective amount of a NEO-201 antibody to said patient.

The name myeloid-derived suppressor cells (MDSCs) was introduced to scientific literature about 15 years ago (Gabrilovich D et al., et al., “The terminology issue for myeloid-derived suppressor cells”, Cancer Res. 2007; 67:42) describing initially a loosely defined group of myeloid cells with potent immune regulatory activity. In recent years, the nature and biological role of MDSC became clearer and MDSCs emerged as a universal regulator of immune function in many pathologic conditions. MDSCs consist of two large groups of cells: granulocytic or polymorphonuclear (PMN-MDSCs or g-MDSCs) and monocytic (m-MDSCs). PMN-MDSCs or g-MDSCs are phenotypically and morphologically similar to neutrophils, whereas m-MDSCs are more similar to monocytes (Gabrilovich D I et al. “Coordinated regulation of myeloid cells by tumors”, Nat Rev Immunol. 2012; 12(4):253-268). Clinical studies in humans have demonstrated the existence of a third small population of MDSCs that are represented by cells with colony forming activity and other myeloid precursors.

Moreover, intensive clinical studies have identified MDSCs as a valuable predictive marker in cancer prognosis. Consequently, as is discussed infra, numerous drugs and treatments are being developed for targeting MDSCs in order to treat diseases wherein such cells are involved in disease pathology.

Morphologically and phenotypically MDSCs are similar to neutrophils and monocytes. The major populations of bone marrow (BM)-derived myeloid cells include granulocytes (with their most abundant representative—neutrophils) and mononuclear cells: monocytes and terminally differentiated macrophages (Mφ) and dendritic cells (DC). In contrast to experiments in vitro, where both Mφ and DCs can be easily differentiated from monocytes, in tissues under steady state conditions, Mφ expand largely in situ and most DCs differentiate from their specific BM precursors (Geissmann F et al., “Development of monocytes, macrophages, and dendritic cells”, Science. 327(5966):656-661). However, during inflammation and cancer, BM-derived monocytes are the primary precursors of Mφ, especially tumor associated macrophages (TAM) and a population of inflammatory DCs (Veglia F et al., “Dendritic cells in cancer: the role revisited”, Curr Opin Immunol. 2017; 45:43-51).

Myeloid cells have emerged in evolution as one of the major protective mechanisms against pathogens and are an important element of tissue remodeling. Under physiological conditions, GM-CSF drives myelopoiesis and G-CSF and M-CSF induce the differentiation of granulocytes and macrophages, respectively (Barreda D R et al., “Regulation of myeloid development and function by colony stimulating factors”, Dev Camp Immunol. 2004; 28(5):509-554). In cancer and in other pathological conditions, these factors are overproduced and favor the generation of MDSC (Gabrilovich D I, et al., “Coordinated regulation of myeloid cells by tumors”, Nat Rev Immunol. 2012; 12(4):253-268; Marvel D, Gabrilovich D I. “Myeloid-derived suppressor cells in the tumor microenvironment: expect the unexpected”, J Clin Invest. 2015; 125(9):3356-3364. Thus, accumulation of MDSCs takes place alongside the same differentiation pathways as neutrophils and monocytes.

Aside from tumors and sites of infection MDSCs can also be detected in the blood, e.g., in some breast cancers, MDSC levels in the blood are about 10-fold higher than normal. (Safarzadeh E, et al., April 2019, “Circulating myeloid-derived suppressor cells: An independent prognostic factor in patients with breast cancer”, Journal of Cellular Physiology. 234 (4): 3515-3525. doi:10.1002/jcp.26896. PMID 30362521.)

The size of the myeloid suppressor compartment is considered to be an important factor in the clinical success or failure of cancer immunotherapy, highlighting the importance of this cell type for human pathophysiology. (Kodach L L, et al., August 2021, “Targeting the Myeloid-Derived Suppressor Cell Compartment for Inducing Responsiveness to Immune Checkpoint Blockade Is Best Limited to Specific Subtypes of Gastric Cancers”, Gastroenterology, 161(2): 727. doi:10.1053/j.gastro.2021.03.047. PMID 33798523.)

As shown infra, Applicant has surprisingly shown that NEO-201 binds to granulocytes and to gMDSCs derived therefrom and specifically elicits the killing or ablation of gMDSCs. Accordingly, NEO-201 potentially may be used in treating any condition where gMDSCs are involved in disease pathology, most particularly cancer and chronic infection conditions where MDSCs are known to suppress innate immunity.

Conditions where NEO-201 May be Used to Ablate gMDSCs

### Cancers

As NEO-201 specifically ablates gMDSCs, NEO-201 can be used in treating any cancer where gMDSCs are impacting innate anticancer immunity. This includes cancers which express the antigen bound by NEO-201 and cancers which do not express the antigens bound by NEO-201.

Accordingly, the types of cancers where MDSCs are involved in disease pathology and wherein NEO-201 may be used to deplete or ablate gMDSCs include both solid tumors and hematological cancers. Exemplary tumors wherein MDSCs may be involved in disease pathology include Adrenal Cancer, Anal Cancer, Bile Duct Cancer, Bladder Cancer, Bone Cancer, Brain/CNS Tumors In Adults, Brain/CNS Tumors In Children, Breast Cancer, Breast Cancer In Men, Cancer in Adolescents, Cancer in Children, Cancer in Young Adults, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Leukemia—Acute Lymphocytic (ALL) in Adults, Leukemia—Acute Myeloid (AML), Leukemia—Chronic Lymphocytic (CLL), Leukemia—Chronic Myeloid (CML), Leukemia—Chronic Myelomonocytic (CMML), Leukemia in Children, Liver Cancer, Lung Cancer, Lung Cancer—Non-Small Cell, Lung Cancer—Small Cell, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Skin Cancer—Basal and Squamous Cell, Skin Cancer—Melanoma, Skin Cancer—Merkel Cell, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor.

In some embodiments such cancer patients can have a cancer and/or a tumor selected from the group consisting of lung cancer, breast cancer, triple negative breast cancer (TNBC), colorectal cancer, liver cancer, stomach cancer, colon cancer, non-small cell lung cancer (NSCLC), bone cancer, pancreatic cancer, skin cancer, head or neck cancer, cutaneous or intraocular melanoma, uterine cancer, ovarian cancer, colorectal cancer, small intestine cancer, rectal cancer, anal cancer, fallopian tube cancer, endometrial cancer, cervical cancer, vaginal cancer, vulva cancer, Hodgkin's disease, esophageal cancer, small intestine cancer, lymph node cancer, bladder cancer, gallbladder cancer, endocrine cancer, thyroid cancer, parathyroid cancer, adrenal cancer, soft tissue sarcoma, urethra cancer, penis cancer, prostate cancer, adenocarcinoma, chronic or acute leukemia, lymphocytic lymphoma, bladder cancer, kidney or ureter cancer, renal cell carcinoma, renal pelvic carcinoma, central nervous system tumor, primary CNS tumor, spinal cord tumor, brainstem glioma, and pituitary adenoma, but is not limited thereto.

Some cancers are strongly influenced by MDSCs and thus, patients affected by these cancers will experience a greater benefit due to the modulation of gMDSC suppressive function and differentiation by the administration of NEO-201. Such a benefit will synergize by inducing a T-cell response, as antigen presentation is improved and other immunosuppressive effects of MDSCs including Treg recruitment is alleviated.

In some aspects of the invention, the cancer patient may have stage I, stage II, stage III, or stage IV cancer involving MDSC. In other aspects, NEO-201 reduces, eliminates or slows or arrests the growth of tumors wherein antitumor immunity is otherwise suppressed by gMDSCs, which can result in reduction in tumor burden in the individual, inhibition of tumor growth, and/or increased survival of the individual.

In some aspects of the invention, the cancer patient may have developed a resistance or tolerance to an anti-cancer treatment, e.g., an immune modulatory antibody, a checkpoint inhibitor antibody or fusion protein, or a chemotherapeutic agent and NEO-201 may be administered alone or in combination with another anti-cancer treatment, e.g., an immune modulatory antibody, a checkpoint inhibitor antibody or fusion protein, or a chemotherapeutic agent in order to reverse such resistance or tolerance.

In some aspects of the invention, the cancer patient may be in remission, and NEO-201 administration may be used in maintenance therapy, e.g., it may be administered alone or in combination with another anti-cancer treatment, e.g., an immune modulatory antibody, a checkpoint inhibitor antibody or fusion protein, or a chemotherapeutic agent in order to suppress cancer reoccurrence by suppressing the proliferation of MDSCs and thereby promoting innate anti-tumor immunity.

In some aspects of the invention, the cancer may have reoccurred, and NEO-201 administration may be administered alone or in combination with another anti-cancer treatment, e.g., an immune modulatory antibody, a checkpoint inhibitor antibody or fusion protein, or a chemotherapeutic agent in order to suppress the proliferation of MDSCs, thereby reestablishing innate anti-tumor immunity.

### Infectious Conditions

Also, NEO-201 potentially may be used in treating bacterial conditions where gMDSCs are involved in disease pathology and may suppress innate immunity.

Types of bacterial infections wherein NEO-201 may be used to deplete gMDSCs include Bacillus anthraces, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis and Enterococcus faecium, Escherichia coli (generally), Enterotoxigenic Escherichia coli (ETEC), Enteropathogenic E. coli, E. coli O157:H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia, Salmonella typhi, Salmonella typhimurium, Shigella sonnei, and Staphylococcus aureus.

Types of viral infections wherein MDSCs reportedly may be involved in disease pathology include both chronic and acute infections. Exemplary infections wherein NEO-201 may be used to deplete gMDSC include Respiratory Viruses, such as, Adenoviruses, Avian influenza, Influenza virus type A, Influenza virus type B, Measles, Parainfluenza virus, Respiratory syncytial virus (RSV), Rhinoviruses, SARS-CoV, Gastro-enteric Viruses, such as, Coxsackie viruses, Enteroviruses, Poliovirus, Rotavirus, Hepatitis Viruses, such as, Hepatitis B virus, Hepatitis C virus, Bovine viral diarrhea virus (surrogate), Herpes Viruses, such as, Herpes simplex 1, Herpes simplex 2, Human cytomegalovirus, Varicella zoster virus, Retroviruses, such as, Human immunodeficiency virus 1 (HIV-1), Human immunodeficiency virus 2 (HIV-2), Simian immunodeficiency virus (SIV), Simian human immunodeficiency virus (SHIV), Viral Select Agents/Emerging Viral Pathogens, such as, Avian influenza, Dengue virus, Hantavirus, Hemorrhagic fever viruses, Lymphocytic choriomeningitis virus, Smallpox virus surrogates, Cowpox, Monkeypox, Rabbitpox, Vaccinia virus, Venezuelan equine encephalomyelitis virus (VEE), West Nile virus, Yellow fever virus.

In some aspects of the invention, the patient may have developed a resistance or tolerance to an anti-infectious agent treatment, e.g., an immune modulatory antibody, a checkpoint inhibitor antibody or fusion protein, or a chemotherapeutic agent and NEO-201 may be administered alone or in combination with another anti-infection treatment, e.g., an immune modulatory antibody, a checkpoint inhibitor antibody or fusion protein, or a chemotherapeutic agent in order to promote innate anti-infection immunity.

In some aspects of the invention, the patient with an infection may be in remission (e.g., a herpes patient), and NEO-201 administration may be used in maintenance therapy, e.g., it may be administered alone or in combination with another anti-infection agent treatment, e.g., an immune modulatory antibody, a checkpoint inhibitor antibody or fusion protein, or a chemotherapeutic agent in order to suppress cancer reoccurrence by suppressing the proliferation of gMDSCs and thereby promoting innate anti-infection immunity.

In some aspects of the invention, the infection may have reoccurred, and NEO-201 administration may be administered alone or in combination with another anti-infection treatment, e.g., an immune modulatory antibody, a checkpoint inhibitor antibody or fusion protein, or a chemotherapeutic agent in order to suppress the proliferation of gMDSCs, thereby reestablishing innate anti-infection immunity.

### Other Conditions Involving MDSCs

Other diseases or conditions wherein MDSCs may be involved in suppressing innate immunity include, but are not limited to: acquired immune deficiency syndrome (AIDS), acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behcet's disease, Berger's disease, Bickerstaff's encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barre syndrome (GBS), Hashimoto's encephalopathy, Hashimoto's thyroiditis, heart disease, Henoch-Schönlein purpura, herpes gestationis (aka gestational pemphigoid), hidradenitis suppurativa, HIV infection, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenic purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosis, linear IgA disease (LAD), lupoid hepatitis (aka autoimmune hepatitis), lupus erythematosus, lymphomatoid granulomatosis, Majeed syndrome, malignancies including cancers (e.g., sarcoma, Kaposi's sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et Varioliformis acuta), multiple sclerosis, myasthenia gravis, myositis, narcolepsy, neuromyelitis optica (aka Devic's disease), neuromyotonia, ocular cicatricial pemphigoid, opsoclonus myoclonus syndrome, Ord's thyroiditis, palindromic rheumatism, PANDAS (pediatric autoimmune neuropsychiatric disorders associated with Streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, sarcoidosis, schizophrenia, Schmidt syndrome, Schnitzler syndrome, scleritis, scleroderma, sepsis, serum Sickness, Sjogren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome,) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

Similarly, NEO-201 administration may be administered alone or in combination with another active agent to patients with such conditions, in order to reestablish, maintain or promote innate immunity.

### Combination Therapies

Combination Therapies with Other Drugs or Biologics

NEO-201 especially should be useful in combination therapies, e.g., in combination with other therapeutics, e.g., other biologics such as therapeutic antibodies and fusion proteins, e.g., those which target cytokines or checkpoint inhibitors, chemotherapeutics, and the like because NEO-201, given its demonstrated ability to deplete gMDSCs should potentiate the efficacy of such other therapeutics, i.e., by restoring or enhancing innate immunity otherwise suppressed by MDSCs, e.g., innate anti-tumor or anti-infectious agent responses, including subjects who previously were resistant to treatment or became resistant to treatment with a particular active.

Particularly provided herein are combination therapies wherein NEO-201 is administered with one or more additional therapeutic agent(s) in order to kill or ablate MDSCs which may inhibit the efficacy of such additional therapeutic agent(s). Such additional therapeutic agent(s) include, without limitation, peptides, nucleic acid molecules, small molecule compounds, antibodies and derivatives thereof.

Combination with Other Drugs that Target MDSCs
i. Combination of NEO-201 with Chemotherapeutics Targeting MDSCs

Diminishing the protumoral effects of MDSCs can be achieved by weakening the immunosuppressive function of MDSCs. STAT3 plays an indispensable role in MDSC-mediated tumorigenesis. By applying a specific small molecule inhibitor of p-STAT3 or STAT3-targeted siRNA to block the activation of STAT3, the suppressive activity of MDSCs can be eliminated by reducing the expression of ARG1 in MDSCs [Vasquez-Dunddel D. et al., “STAT3 regulates arginase-I in myeloid-derived suppressor cells from cancer patients”, J. Clin. Invest. 2013; 123:1580-1589, Trovato R. et al., “Immunosuppression by monocytic myeloid-derived suppressor cells in patients with pancreatic ductal carcinoma is orchestrated by STAT3”, J. Immunother. Cancer. 2019; 7:255]. Receptor tyrosine kinases, such as TYRO3 (a type of protein tyrosine kinase), AXL (a type of receptor tyrosine kinase), and C-Mer proto-oncogene tyrosine kinase (MERTK) and their ligands, Gas 6 and Protein S, can reverse the tumorigenic properties of MDSCs, increase the numbers of tumor infiltrating CD8+ T cells, and strengthen anti-PD-1 immune checkpoint therapy. MERTK abolishes the suppressive capability of MDSCs by negatively regulating STAT3 [Holtzhausen A. et al., “TAM family receptor kinase inhibition reportedly reverses MDSC-mediated suppression and augments Anti-PD-1 therapy in melanoma”, Cancer Immunol. Res. 2019. Moreover, STAT3 inhibitors, such as sunitinib, AZD9150, and BB1608, or a conjugate of the STAT3 antisense oligonucleotide (ASO) tethered to immunostimulatory toll-like receptor9 (TLR9) agonist (CpG-STAT3ASO) conjugates reportedly can significantly diminish the immunosuppressive function of MDSCs and rescue antitumor immunity [Guha P., et al., “STAT3 inhibition induces Bax-dependent apoptosis in liver tumor myeloid-derived suppressor cells”, Oncogene. 2019; 38:533-548.; Moreira D, et al. “STAT3 inhibition combined with CpG immunostimulation activates antitumor immunity to eradicate genetically distinct castration-resistant prostate cancers”, Clin. Cancer Res. 2018; 24:5948-5962; Reilley M. J., et al. “STAT3 antisense oligonucleotide AZD9150 in a subset of patients with heavily pretreated lymphoma: Results of a phase 1b trial”, J. Immunother. Cancer. 2018; 6:119].

PGE2 reportedly induces MDSCs to upregulate the production of ARG1 and iNOS and exert suppression. Cyclooxygenase-2 (COX-2) is the upstream molecular signal of PGE2, which regulates the generation of PGE2. Thus, COX-2 can be targeted to negatively regulate the synthesis of PGE2. shRNA targeting of COX-2 significantly reduces MDSCs in the spleens of tumor-bearing mice [Mao Y., et al., “Inhibition of tumor-derived prostaglandin-e2 blocks the induction of myeloid-derived suppressor cells and recovers natural killer cell activity”, Clin. Cancer Res. 2014; 20:4096-4106]. COX-2 expression can also be inhibited by acetylsalicylic acid, NS-398, and celecoxib, thereby hindering the activity of MDSCs and increasing the infiltration of CTLs in tumor sites [Wong J. L. et al., “Synergistic COX2 Induction by IFNgamma and TNFalpha Self-Limits Type-1 Immunity in the Human Tumor Microenvironment”, Cancer Immunol. Res. 2016; 4:303-311, Chen W. C. et al., “Inflammation-induced myeloid-derived suppressor cells associated with squamous cell carcinoma of the head and neck”, Head Neck. 2017; 39:347-355, Fujita M. et al., “COX-2 blockade suppresses gliomagenesis by inhibiting myeloid-derived suppressor cells”, Cancer Res. 2011; 71:2664-2674.].

RIPK3 induces cell necrosis by interacting with TLR3/4 [He S. et al., “Toll-like receptors activate programmed necrosis in macrophages through a receptor-interacting kinase-3-mediated pathway”, Proc. Natl. Acad. Sci. USA. 2011; 108:20054-20059.]. RIPK3 deficiency activates the NF-κB signaling pathway and upregulates the expression of the downstream signaling molecules COX-2 and PGE2, which aggravates the immunosuppressive activity of MDSCs and accelerates tumor growth. Treatment with aspirin (ASA, COX inhibitor) reportedly significantly protected mice against tumorigenesis [Yan G. et al., “A RIPK3-PGE2 circuit mediates myeloid-derived suppressor cell-potentiated colorectal carcinogenesis”, Cancer Res. 2018; 78:5586-5599.]. Additionally, the overexpression of fatty acid transport protein 2 (FATP2) is also involved in the synthesis of PGE2 through the activation of the STAT5 signaling pathway. Administration of the selective FATP2 inhibitor lipofermata selectively inhibits the function of MDSCs while enhancing immunotherapy [Veglia F. et al., “Fatty acid transport protein 2 reprograms neutrophils in cancer”, Nature. 2019; 569:73-78. doi: 10.1038/s41586-019-1118-2.].

Phosphodiesterase 5 (PDE5) is another target of MDSC treatment that is a hydrolase that acts on the NO/cyclic guanosine monophosphate (cGMP) signaling pathway [Peak T. C. et al., “The Role of PDE5 inhibitors and the NO/cGMP pathway in cancer”, Sex. Med. Rev. 2016; 4:74-84.]. The application of PDE5 inhibitors, including sildenafil, tadalafil, and vardenafil, can reduce the production of ARG1 and iNOS in MDSCs, abolish the inhibitory activity of MDSCs, reduce the number of Tregs, and thus greatly delay the progression of tumors [Tai L. H. et al., “Phosphodiesterase-5 inhibition reduces postoperative metastatic disease by targeting surgery-induced myeloid derived suppressor cell-dependent inhibition of Natural Killer cell cytotoxicity”, OncoImmunology. 2018; 7:e1431082. doi: 10.1080/2162402X.2018.1431082, Weed D. T. et al., “Tadalafil reduces myeloid-derived suppressor cells and regulatory T cells and promotes tumor immunity in patients with head and neck squamous cell carcinoma”, Clin. Cancer Res. 2015; 21:39-48, Noonan K. A. et al., “Targeting immune suppression with PDE5 inhibition in end-stage multiple myeloma” Cancer Immunol. Res., 2014; 2:725-731, Serafini P. et al., “Phosphodiesterase-5 inhibition augments endogenous antitumor immunity by reducing myeloid-derived suppressor cell function”, J. Exp. Med., 2006; 203:2691-270]. Treatment with tadalafil combined with cytokine-induced killer (CIK) cell-based immunotherapy reportedly enhanced CIK activity against human hepatocellular carcinoma (HCC) cell lines in vitro [Yu S. J. et al., “Targeting the crosstalk between cytokine-induced killer cells and myeloid-derived suppressor cells in hepatocellular carcinoma”, J. Hepatol., 2019; 70:449-457]. Nitroaspirin is another inhibitor of ARG1 and iNOS that reduces ROS generation [De Santo C. et al., “Nitroaspirin corrects immune dysfunction in tumor-bearing hosts and promotes tumor eradication by cancer vaccination”, Proc. Natl. Acad. Sci. USA. 2005; 102:4185-4190.].

Nuclear factor E2-related factor 2 (Nrf2), a transcription factor, reportedly is the main regulator of antioxidant stress. Nrf2 is associated with abnormal ROS accumulation in MDSCs, which has been confirmed by a model of Nrf2-deficient mice. In Nrf2 knockout (KO) mice, the circulating level of MDSCs did not change; however, with elevated amounts of cellular ROS, the number of CD8+ T cells was significantly reduced, and the tumor growth rate increased [Satoh H. et al., “Nrf2-deficiency creates a responsive microenvironment for metastasis to the lung”, Carcinogenesis. 2010; 31:1833-1843, Zhang D. et al., “Identification of an unfavorable immune signature in advanced lung tumors from Nrf2-deficient mice. Antioxid. Redox Signal. 2018; 29:1535-1552.]. Treatment with Nrf2-inducing triterpenoids, such as omaveloxolone (RTA-408), CDDO-Me (RTA-402), and CDDO-Im (RTA-403), reportedly increases the transcriptional activity of Nrf2, which attenuates the production of ROS, abrogates the immune suppressive effect of MDSCs, and protects immune cells and tissues from oxidative stress [Hiramoto K. et al., “Myeloid lineage-specific deletion of antioxidant system enhances tumor metastasis”, Cancer Prev. Res. (Phila.) 2014; 7:835-844, Creelan B. et al., “Safety, pharmacokinetics, and pharmacodynamics of oral omaveloxolone (RTA 408), a synthetic triterpenoid, in a first-in-human trial of patients with advanced solid tumors”, OncoTargets Ther. 2017; 10:4239-4250, Nagaraj S. et al., Youn J. “Anti-inflammatory triterpenoid blocks immune suppressive function of MDSCs and improves immune response in cancer”, Clin. Cancer Res. 2010; 16:1812-1823]. However, a recent study has demonstrated that Nrf2 is activated by PKR-like endoplasmic reticulum (ER) kinase (PERK) in tumor-infiltrating MDSCs, giving MDSCs the potential for immunosuppression [Mohamed E. et al., “The unfolded protein response mediator perk governs myeloid cell-driven immunosuppression in tumors through inhibition of STING signaling”, Immunity, 2020; 52:668-682.]. The deletion of PERK or treatment with the selective inhibitor of PERK AMG-44 reportedly reduces Nrf2 transcription, resulting in ROS overexpression, causing mitochondrial damage, impeding the immunosuppression of MDSCs, and increasing the infiltration of CD8⁺ T cells. This situation can be antagonized by the addition of the Nrf2 inducer sulforaphane [Mohamed E. et al., “The unfolded protein response mediator perk governs myeloid cell-driven immunosuppression in tumors through inhibition of STING signaling”, Immunity. 2020; 52:668-682]. Based on the foregoing, Nrf2 overexpression and deletion affect the immunoinhibitory activity of MDSCs and only when Nrf2 maintains a steady state can MDSCs exert normal protumor effects.

N-Hydroxy-nor-L-arginine (nor-NOHA) is used as an ARG1 inhibitor. Blocking ARG1 by nor-NOHA reportedly reversed the immunosuppressive activity of MDSCs [Bak S. P. et al “Murine ovarian cancer vascular leukocytes require arginase-1 activity for T cell suppression”, Mol. Immunol. 2008; 46:258-268]. Inhibition of the VEGF/VEGFR-2 axis with antibody DC101 repressed primary tumor growth and metastasis in the 4T1 breast cancer model. Also, arginase inhibition reportedly suppresses lung metastasis in the 4T1 breast cancer model independently of the immunomodulatory and anti-metastatic effects of VEGFR-2 blockade. OncoImmunology. 2017; 6:e1316437.]. 1-Methyl-DLtryptophan (1-MT), a competitive inhibitor of IDO, reportedly ablates the immunosuppressive function of MDSCs on T cells. When 1-MT is combined with nor-NOHA, the T cell proliferation rate is almost completely restored [Du J. et al., “The study of CD14+HLA-DR-/low myeloid-derived suppressor cell (MDSC) in peripheral blood of peripheral T-cell lymphoma patients and its biological function” Cell. Mol. Biol. 2017; 63:62-67.].

Bruton's tyrosine kinase (BTK) reportedly is a nonreceptor intracellular kinase that is related to the migration and proliferation of MDSCs. Treatment with the BTK inhibitory drug ibrutinib decreases the cytokine production and motility of MDSCs [Molina-Cerrillo J. et al., “Bruton's tyrosine kinase (BTK) as a promising target in solid tumors”, Cancer Treat. Rev. 2017; 58:41-50.].

Also it has been reported that estrogen interacts with estrogen receptor alpha, driving the mobilization of MDSCs by activating the STAT3 pathway, which facilitates deregulated myelopoiesis. The progression of tumors can be delayed by removing estrogen activity though an anti-estrogen treatment [Svoronos N. et al., “Tumor cell-independent estrogen signaling drives disease progression through mobilization of myeloid-derived suppressor cells”, Cancer Discov. 2017; 7:72-85]. Castration-resistant prostate cancer exhibits resistance to androgen deprivation therapy mainly because IL-23 secreted by MDSCs activates the androgen receptor (AR) and the STAT3/RORγ signaling axis in prostate tumor cells. Blocking the production of IL-23 can counteract MDSC-mediated CRPC through treatment with the anti-IL-23 antibody and AR antagonist enzalutamide [Calcinotto A. et al., “IL-23 secreted by myeloid cells drives castration-resistant prostate cancer”, Nature. 2018; 559:363-369].

MDSCs have low glycolysis and mitochondrial respiratory capacity but contain high levels of methylglyoxal, which inhibits the antitumor activity of CD8+ effector T cells. Neutralization of methylglyoxal with compounds containing guanidine groups, such as metformin, can effectively abolish the immunosuppressive activity of MDSCs. The combination of metformin and anti-PD-1 overcomes the suppression of immunotherapy by MDSCs [Baumann T. et al., “Regulatory myeloid cells paralyze T cells through cell-cell transfer of the metabolite methylglyoxal”, Nat. Immunol. 2020; 21:555-566.].

ii. Combination of NEO-201 with other Drugs that Deplete MDSCs Treatment with low doses of chemotherapy drugs, such as gemcitabine, 5-fluorouracil (5-FU), paclitaxel, and cisplatin, effectively affects the viability of MDSCs [Wang Y. et al., “Metabolic regulation of myeloid-derived suppressor cell function in cancer”, Cells, 2020; 9:1011, Won W. J. et al., “Metabolic and functional reprogramming of myeloid-derived suppressor cells and their therapeutic control in glioblastoma”, Cell Stress. 2019; 3:47-65 Chaib M. et al., “Friend or foe?Recent strategies to target myeloid cells in cancer”, Front. Cell Dev. Biol. 2020; 8:351.]. Gemcitabine is a selective inhibitor of MDSCs that reduces the number of circulating Tregs and the level of TGFβ1 and PMN-MDSCs but not M-MDSCs in the peripheral blood of patients with pancreatic cancer and restores the proliferation and antitumor capacity of effector T cells [Eriksson E. et al., “Gemcitabine reduces MDSCs, Tregs and TGFbeta-1 while restoring the Teff/Treg ratio in patients with pancreatic cancer”, J. Transl. Med. 2016; 14:282]. 5-FU can equally induce the death of the two subtypes of MDSCs and has no obvious effect on other immune cells, such as T cells, NK cells, DCs, and B cells. Treatment with 5-FU reportedly triggered the apoptosis of MDSCs, promoted tumor-infiltrating T cells to produce high levels of IFNγ and enhanced the T cell-dependent antitumor response in the mouse EL4 model [incent J. et al., “5-Fluorouracil selectively kills tumor-associated myeloid-derived suppressor cells resulting in enhanced T cell-dependent antitumor immunity”, Cancer Res. 2010; 70:3052-3061]. 5-FU reportedly significantly and specifically eliminated MDSCs by inducing apoptosis in the TME and spleen of tumor-bearing mice [Vincent J. et al., “5-Fluorouracil selectively kills tumor-associated myeloid-derived suppressor cells resulting in enhanced T cell-dependent antitumor immunity”, Cancer Res. 2010; 70:3052-3061]. However, the assembly of NLRP3 in MDSCs is activated by 5-FU, which reportedly leads to the secretion of MDSC-derived IL-1β and CD4+ T cell-derived IL-17 and inhibits the antitumor effect of 5-FU. Based thereon, combined administration of 5-FU and IL-1β inhibitors, such as the indirect inhibitors DHA and SP600125, could provide an effective means for inhibiting MDSCs [Dumont A. et al., “Docosahexaenoic acid inhibits both NLRP3 inflammasome assembly and JNK-mediated mature IL-1beta secretion in 5-fluorouracil-treated MDSC: Implication in cancer treatment”, Cell Death Dis. 2019; 10:485, Bruchard M. et al., “Chemotherapy-triggered cathepsin B release in myeloid-derived suppressor cells activates the Nlrp3 inflammasome and promotes tumor growth”, Nat. Med. 2013; 19:57-64]. Docetaxel, which has the same effect as paclitaxel, was reported to significantly inhibit tumor growth. Docetaxel achieves its antitumor effect by polarizing MDSCs to M1-type macrophages, reducing the proportion of MDSCs in the spleen [Kodumudi K. N. et al., “A novel chemoimmunomodulating property of docetaxel: Suppression of myeloid-derived suppressor cells in tumor bearers”, Clin. Cancer Res. 2010; 16:4583-4594.]. ApoE impedes tumor invasion and endothelial cell recruitment, but liver-X receptors (LXRs) inhibit ApoE expression. It has been reported that the LXR agonists GW3965 and RGX-104 impair MDSC survival by activating the LXR/ApoE axis and enhance the antitumor activity of CTLs [Tavazoie M. F. et al., “LXR/ApoE Activation Restricts Innate Immune Suppression in Cancer”, Cell. 2018; 172:825-840, Liang H. et al., “LXR activation radiosensitizes non-small cell lung cancer by restricting myeloid-derived suppressor cells”, Biochem. Biophys. Res. Commun. 2020; 528:330-335]. CD33 is highly expressed on MDSCs in humans, especially M-MDSCs, but CD33 is a therapeutic target on circulating and tumor-infiltrating MDSCs across multiple cancer types [Lamba J. K. et al., “CD33 splicing polymorphism determines gemtuzumab ozogamicin response in de novo acute myeloid leukemia: Report from randomized phase 11 children's oncology group trial AAML0531”, J. Clin. Oncol. Off. J. Am. Soc. Clin. Oncol. 2017; 35:2674-2682]. The immunotoxin gemtuzumab ozogamicin, a CD33 monoclonal antibody (mAb), effectively eliminates MDSCs and reactivates T cells to fight against multiple cancers [Lamba J. K. et al “CD33 splicing polymorphism determines gemtuzumab ozogamicin response in de novo acute myeloid leukemia: Report from randomized phase III children's oncology group trial AAML0531”, J. Clin. Oncol. Off. J. Am. Soc. Clin. Oncol. 2017; 35:2674-2682, Fultang L. et al., “MDSC targeting with Gemtuzumab ozogamicin restores T cell immunity and immunotherapy against cancers”, EBioMedicine. 2019; 47:235-246.]. Additionally, targeting the bromodomain and extraterminal domain (BET), a component of the endogenous transcription enhancer of MDSCs, by treating HCC patient-derived PBMCs with the small molecular inhibitor i-BET762 reportedly significantly reduced the number of CD14+HLA-DR-/low M-MDSCs and enhanced the effect of immunotherapy [Liu M. et al., “Targeting monocyte-intrinsic enhancer reprogramming improves immunotherapy efficacy in hepatocellular carcinoma”, Gut. 2020; 69:365-379.].

iii. Combination of NEO-201 with Drugs that Block MDSC Migration

Blocking the migration of MDSCs reportedly can effectively reduce the proportion of MDSCs in the TME and the periphery by impeding the response of MDSCs to chemokines [De Sanctis F. et al., “MDSCs in cancer: Conceiving new prognostic “The tumor microenvironment innately modulates cancer progression”, Cancer Res. 2019; 79:4557-4566]. Antagonists of chemokines reportedly can help prevent MDSCs, especially PMN-MDSCs, from reaching the tumor sites and modifying the immunosuppressive microenvironment [Zhou J et al., “Neutrophils and PMN-MDSC: Their biological role and interaction with stromal cells”, Semin. Immunol. 2018; 35:19-28]. CXCR2 is an important chemokine receptor for MDSC trafficking [Park S. M et al., “Role of myeloid-derived suppressor cells in immune checkpoint inhibitor therapy in cancer”, Arch. Pharm. Res. 2019; 42:560-566, Cheng Y et al., “Potential roles and targeted therapy of the CXCLs/CXCR2 axis in cancer and inflammatory diseases”, Biochim. Biophys. Acta Rev. Cancer. 2019; 1871:289-312.]. Blocking the CXCR2/CXCLs pathway through CXCR2 inhibitors, such as SX-682, reparixin, and SB225002, reportedly effectively reduces the infiltration of MDSCs and improves the function of cytotoxic T cells [Yan G et al., “A RIPK3-PGE2 circuit mediates myeloid-derived suppressor cell-potentiated colorectal carcinogenesis”, Cancer Res. 2018; 78:5586-5599, Liao W et al., “KRAS-IRF2 axis drives immune suppression and immune therapy resistance in colorectal cancer”, Cancer Cell. 2019; 35:559-572, Ocana A., et al., “Neutrophils in cancer: Prognostic role and therapeutic strategies, Mol. Cancer. 2017; 16:137. doi: 10.1186/s12943-017-0707-7.]. The progression and invasiveness of multiple tumors reportedly can be suppressed by targeting the CCR5/CCL axis [Tan M. C. et al., “Disruption of CCR5-dependent homing of regulatory T cells inhibits tumor growth in a murine model of pancreatic cancer”, J. Immunol. 2009; 182:1746-1755, Zhang X. et al., “Anibamine, a natural product CCR5 antagonist, as a novel lead for the development of anti-prostate cancer agents”, Bioorganic Med. Chem. Lett. 2010; 20:4627-4630,Velasco-Velazquez M. et al., “CCR5 antagonist blocks metastasis of basal breast cancer cells”, Cancer Res. 2012; 72:3839-3850; Halama N. et al., “Tumoral immune cell exploitation in colorectal cancer metastases can be targeted effectively by Anti-CCR5 therapy in cancer patients”, Cancer Cell. 2016; 29:587-60]. Administration of mCCR5-Ig-neutralizing CCR5 ligands reportedly reduced the migration of MDSCs and Tregs without impacting the recruitment of effector T cells to the TME [Blattner C. et al., “CCR5(+) myeloid-derived suppressor cells are enriched and activated in melanoma lesions”, Cancer Res. 2018; 78:157-167]. The CXCR4 receptor for CXCL12 (also known as stromal cell-derived factor 1, SDF-1) also mediates the recruitment of MDSCs. Neutralization of CXCR4 by antagonists, such as AMD3100, reportedly reduces the number of MDSCs and Tregs and promotes M2 to M1 macrophage polarization in the TME [Wang J. et al., “CXCR4 antagonist AMD3100 (plerixafor): “From an impurity to a therapeutic agent”, Pharmacol. Res. 2020:105010, Zhuang Y. et al., “CD8(+) T cells that produce interleukin-17 regulate myeloid-derived suppressor cells and are associated with survival time of patients with gastric cancer”, Gastroenterology, 2012; 143:951-962.]. Moreover, the colony-stimulating factor-1 receptor (CSF-1R) is a tyrosine kinase receptor that, when combined with the receptor, reportedly can induce the formation of MDSCs and trafficking to tumor sites. It has recently been reported that CSF-1R inhibitors, such as RG7155 and PLX647, block the CSF-1R signaling pathway, reportedly leading to ablation of MDSCs or inhibition of their tumor-promoting functions and reprogramming of TAMs [Law A. M. K. et al., “Myeloid-derived suppressor cells as a therapeutic target for cancer”, Cells 2020; 9:561, Holmgaard R. B. “Targeting myeloid-derived suppressor cells with colony stimulating factor-1 receptor blockade can reverse immune resistance to immunotherapy in indoleamine 2,3-dioxygenase-expressing tumors”, EBioMedicine, 2016; 6:50-58, Mitchem J. B. et al., “Targeting tumor-infiltrating macrophages decreases tumor-initiating cells, relieves immunosuppression, and improves chemotherapeutic responses”, Cancer Res. 2013; 73:1128-1141, Lonardi S. et al., “Potential contribution of tumor-associated slan(+) cells as anti-CSF-1R targets in human carcinoma”, J. Leukoc. Biol. 2018; 103:559-564.].

iv. Combination of NEO-201 with Compounds that Induce MDSC Differentiation

Another means for targeting MDSCs is by inducing MDSCs to differentiate into cells with a proinflammatory phenotype. All-trans retinoic acid (ATRA) is a metabolic intermediate of vitamin A and has been identified as an anticancer drug that induces MDSCs to differentiate into DCs and macrophages [Fleming V. et al., “Targeting myeloid-derived suppressor cells to bypass tumor-induced immunosuppression, Front. Immunol. 2018; 9:398, Nefedova Y. et al., “Mechanism of all-trans retinoic acid effect on tumor-associated myeloid-derived suppressor cells”, Cancer Res. 2007; 67:11021-11028, Schneider A. K. et al., “The multifaceted immune regulation of bladder cancer”, Nat. Rev. Urol. 2019; 16:613-630.]. ATRA reportedly induces the differentiation of MDSCs both in vivo and in vitro, which reportedly greatly reduces the number of MDSCs. The supposed specific mechanism is that the added ATRA activates the ERK1/2 signal, which further upregulates the expression of glutathione synthase in MDSCs, resulting in increased glutathione levels, neutralization of the generated ROS, and inhibition of MDSC inhibitory activity [Ohl K. et al., “Reactive oxygen species as regulators of MDSC-mediated immune suppression”, Front. Immunol. 2018; 9 doi: 10.3389/fimmu.2018.02499]. Further, myeloid cells reportedly differentiate in response to treatment with ATRA. Additionally, vitamin D3 reportedly may also promote the differentiation of MDSCs. MDSCs at the tumor site have higher levels of vitamin D receptor compared with those in the spleen and bone marrow. Treatment with the active form of vitamin D3 (1α,25-dihydroxyvitamin D3,1,25(OH)D) reportedly significantly reduced the T cell suppressive capacity of MDSCs. In vitro-derived MDSCs reduced the production of NO under the stimulation of 1,25(OH)D [Fleet J. C. et al., “1alpha, 25 Dihydroxyvitamin D (1,25(OH)2D) inhibits the T cell suppressive function of myeloid derived suppressor cells (MDSC)”, J. Steroid Biochem. Mol. Biol. 2020; 198:105557]. Another study reported that the addition of 1,25(OH)D abolished the accumulation of IL-6-induced MDSCs [Chen P. T. et al., “1alpha,25-Dihydroxyvitamin D3 Inhibits esophageal squamous cell carcinoma progression by reducing IL6 Signaling”, Mol. Cancer Ther. 2015; 14:1365-1375.]. Based on the foregoing, combining NEO-201 and therapies targeting MDSCs should further reduce the number and function of MDSCs at tumor sites and the circulation.

Combination of NEO-201 with Epigenetic Therapy

Epigenetic therapy is another reported method of targeting MDSCs to treat cancer. Reported epigenetic therapeutic approaches mainly include treatment with histone methyltransferase inhibitors (HMTis), histone deacetylase inhibitors (HDACis), and DNA methyltransferase inhibitors (DNMTis) [Gomez S. et al., “Combining epigenetic and immune therapy to overcome cancer resistance”, Semin. Cancer Biol. 2019]. Enhancer of zeste homolog 2 (EZH2), a gene encoding histone methyltransferase, is often overexpressed in multiple cancer types [Zhou J., et al. “Targeting EZH2 histone methyltransferase activity alleviates experimental intestinal inflammation”, Nat. Commun. 2019; 10:2427.]. After treatment with the EZH2 inhibitor GSK343, the number of functional MDSCs reportedly increased significantly in colorectal cancer mouse models or in vitro [156]. Similarly, the use of another inhibitor, GSK126, also promoted the proliferation of MDSCs. Anti-Gr1 antibody or gemcitabine/5-FU combined with GSK126 can relieve the immunosuppression of MDSCs and increase the number of tumor-infiltrating T cells [Huang S., Wang Z., Zhou J., Huang J., Zhou L., Luo J., Wan Y. Y., Long H., Zhu B. EZH2 inhibitor GSK126 suppresses antitumor immunity by driving production of myeloid-derived suppressor cells. Cancer Res. 2019; 79:2009-2020]. HDAC2 silences the transcription of the retinoblastoma (Rb) gene through epigenetic modification; thus, M-MDSCs acquire partial phenotypes and functions of PMN-MDSCs in tumor-bearing mice [Youn J. I., Kumar V., Collazo M., Nefedova Y., Condamine T., Cheng P., Villagra A., Antonia S., McCaffrey J. C., Fishman M., et al. Epigenetic silencing of retinoblastoma gene regulates pathologic differentiation of myeloid cells in cancer”, Nat. Immunol. 2013; 14:211-220]. DNMTi 5-azacytidine (AZA) reportedly increases the proportion of CD8+ T cells and NK cells in the TME through a type I IFN immune response, reduces the accumulation of MDSCs, and promotes antitumor effects. The addition of an HDACi entinostat (ENT) to AZA reportedly further enhances the regulation of the immune microenvironment. Triple or quadruple treatment of AZA and ENT plus immunotherapy anti-PD-1 and anti-CTLA-4 exhibited highly effective tumor elimination [Stone M. L. et al., “Epigenetic therapy activates type I interferon signaling in murine ovarian cancer to reduce immunosuppression and tumor burden”, Proc. Natl. Acad. Sci. USA. 2017; 114:E10981-E10990, Kim K. et al., “Eradication of metastatic mouse cancers resistant to immune checkpoint blockade by suppression of myeloid-derived cells”, Proc. Natl. Acad. Sci. USA. 2014; 111:11774-11779, Lu Z., et al. “Epigenetic therapy inhibits metastases by disrupting premetastatic niches”, Nature. 2020; 579:284-290, Zhang Z. et al., “Glucocorticoids promote the onset of acute experimental colitis and cancer by upregulating mTOR signaling in intestinal epithelial cells”, Cancers. 2020; 12:945.]. Adjuvant epigenetic therapy with AZA and ENT reportedly blocks the migration of MDSCs by downregulating CCR2 and CXCR2, which leads to the differentiation of MDSCs into macrophages and disturbance of pMN [Lu Z., et al., “Epigenetic therapy inhibits metastases by disrupting premetastatic niches”, Nature. 2020; 579:284-290, Wang X., Bi Y., et al., “The calcineurin-NFAT axis controls allograft immunity in myeloid-derived suppressor cells through reprogramming T cell differentiation”, Mol. Cell. Biol. 2015; 5:598-609, Liu G. et al., “SIRT1 limits the function and fate of myeloid-derived suppressor cells in tumors by orchestrating HIF-1α-dependent glycolysis”, Cancer Res. 2014; 74:727-737.].

Combination with Immune Checkpoint Inhibitors

In some embodiments, the additional therapeutic agent administered with the NEO-201 antibody is an immune checkpoint inhibitor. In some embodiments, the immune checkpoint inhibitor is an anti-PD-1 antibody, an anti-PD-L1 antibody, an anti-CTLA-4 antibody, an anti-CD28 antibody, an anti-TIGIT antibody, an anti-LAGS antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments the additional therapeutic agent is an anti-LAG-3 antibody selected from the group consisting of: BMS-986016 and LAG525. In some embodiments, the additional therapeutic agent is an anti-OX-40 antibody selected from: MED16469, MED10562, and MOXR0916. In some embodiments, the additional therapeutic agent is the anti-4-1BB antibody PF-05082566.

In some embodiments; the additional therapeutic agent is one that targets an immune checkpoint. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (AZAR), B7-H3 (also known as CD276); B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example, it is known that lambrolizumab is also known under the alternative and equivalent names MK-3475 and pembrolizumab.

It is contemplated that any of the immune checkpoint inhibitors that are known in the art which stimulate immune responses may be used. This includes inhibitors that directly or indirectly stimulate or enhance antigen-specific T-lymphocytes. These immune checkpoint inhibitors include, without limitation, agents targeting immune checkpoint proteins and pathways involving PD-L2, LAG3, BTLA, B7H4 and TIM3. For example, LAG3 inhibitors known in the art include soluble LAG3 (IMP321, or LAG3-Ig disclosed in WO2009044273) as well as mouse or humanized antibodies blocking human LAG3 (e.g., IMP701 disclosed in WO2008132601), or fully human antibodies blocking human LAG3 (such as disclosed in EP 2320940). Another example is provided by the use of blocking agents towards BTLA, including without limitation antibodies blocking human BTLA interaction with its ligand (such as 4C7 disclosed in WO2011014438). Yet another example is provided by the use of agents neutralizing B7H4 including without limitation antibodies to human B7H4 (disclosed in WO 2013025779, and in WO2013067492) or soluble recombinant forms of B7H4 (such as disclosed in US20120177645). Yet another example is provided by agents neutralizing B7-H3, including without limitation antibodies neutralizing human B7-H3 (e.g. MGA271 disclosed as BRCA84D and derivatives in US 20120294796). Yet another example is provided by agents targeting TIM3, including without limitation antibodies targeting human TIM3 (e.g. as disclosed in WO 2013006490 A2 or the anti-human TIM3, blocking antibody F38-2E2 disclosed by Jones et al., J Exp Med. 2008; 205(12):2763-79).

In addition, more than one immune checkpoint inhibitor (e.g., an anti-PD-1 antibody and anti-CTLA-4 antibody) may be used in combination with NEO-201. For example, p53 gene therapy and immune checkpoint inhibitors (e.g., anti-MR antibody and/or anti-PD-1 antibody) can be administered to enhance innate anti-tumor immunity followed by IL24 gene therapy and immune checkpoint inhibitors (e.g., anti-PD-1 antibody) to induce adaptive anti-tumor immune responses.

Specifically embraced by the invention are methods for treating or delaying progression of cancer in an individual involving MDSC immunosuppression comprising administering to the individual an effective amount of a PD-1 axis binding antagonist in combination with NEO-201. For example, a PD-1 axis binding antagonist includes a PD-1 binding antagonist, a PD-L1 binding antagonist and a PD-L2 binding antagonist.

In some embodiments, the PD-1 binding antagonist is a molecule that inhibits the binding of PD-1 to its ligand binding partners. In a specific aspect, the PD-1 ligand binding partners are and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, the PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesion, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO is an anti-PD-1 antibody described in WO2006/121168. Pembrolizumab, also known as MK-3475, Merck 3475, lambrolizumab, KEYTRUDA, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 binding antagonists include Pidilizumab, also known as CT-011, MEDI0680, also known as AMP-514, and REGN2810.

In some aspects, the immune checkpoint inhibitor is a PD-L1 antagonist such as Durvalumab, also known as MED14736, atezolizumab, also known as MPDL3280A, or avelumab, also known as MSB00010118C. In certain aspects, the immune checkpoint inhibitor is a PD-L2 antagonist such as rHIgM12B7. In some aspects, the immune checkpoint inhibitor is a LAG-3 antagonist such as, but not limited to, IMP321, and BMS-986016. The immune checkpoint inhibitor may be an adenosine A2a receptor (A2aR) antagonist such as PBF-509.

Another immune checkpoint that may be potentiated by NEO-201's ablative effect on MDSCs is the cytotoxic T-lymphocyte-associated protein 4 (CTLA-4), also known as CD152. The complete cDNA sequence of human CTLA-4 has the GenBank accession number L15006. CTLA-4 is found on the surface of T cells and acts as an “off” switch when bound to CD80 or CD86 on the surface of antigen-presenting cells. CTLA-4 is a member of the immunoglobulin superfamily that is expressed on the surface of Helper T cells and transmits an inhibitory signal to T cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint that may be potentiated by NEO-201's ablative effect on gMDSC is a CSF-1/1R binding agent or inhibitor (e.g. an anti-CSF1 or anti-CSF1R antibody), where the combination is used to treat a cancer, e.g., a cancer described herein, e.g., a solid tumor involving MDSCs. In certain embodiments, the CSF-1/1R binding agent is a CSF-1R tyrosine kinase inhibitor, 4-((2-(((1R,2R)-2-hydroxycyclohexyl)amino)benzo[d]thiazol-6-yl)oxy)-N-met-hylpicolinamide (Compound A15), or a compound disclosed in PCT Publication No. WO 2005/073224. In certain embodiments, the CSF-1/1R binding agent is an M-CSF inhibitor, Compound A33, or a binding agent to CSF-1 disclosed in PCT Publication No. WO 2004/045532 or PCT Publication No WO 2005/068503 including RX 1 or 5H4 (e.g., an antibody molecule or Fab fragment against M-CSF). In certain embodiments, the CSF-1/1R binding agent is 4-(2-((1R, 2R)-2-hydroxycyclohexylamino)benzothiazol-6-yloxy)-N-methylpicolinamide, or BLZ-945. 4-(2-((1R, 2R)-2-hydroxycyclohexylamino)benzothiazol-6-yloxy)-N-methylpicolinamide is disclosed as example 157 at page 117 of PCT Publication No. WO 2007/121484. In certain embodiments, the CSF-1/1R binding agent is pexidartinib (CAS Registry Number 1029044-16-3). Pexidrtinib is also known as PLX3397 or 5-((5-chloro-1H-pyrrolo[2,3-b]pyridin-3-yl)methyl)-N-((6-(trifluoromet-hy-l)pyridin-3-yl)methyl)pyridin-2-amine. Pexidartinib is a small-molecule receptor tyrosine kinase (RTK) inhibitor of KIT, CSF1R and FLT3. In certain embodiments, the CSF-1/1R binding agent is emactuzumab. Emactuzumab is also known as RG7155 or R05509554. Emactuzumab is a humanized IgG1 mAb targeting CSF1R. In certain embodiments, the CSF-1/1R binding agent is FPA008. FPA008 is a humanized mAb that inhibits CSF1R.

Other therapeutic agent wherein the combination thereof with NEO-201 may elicit synergistic effects in treating conditions wherein MDSCs are involved in pathology include (a) microtubule inhibitors, topoisomerase inhibitors, platins, alkylating agents, and anti-metabolites; (b) MK-2206, ON 013105, RTA 402, BI 2536, Sorafenib, ISIS-STAT3Rx, a microtubule inhibitor, a topoisomerase inhibitor, a platin, an alkylating agent, an anti-metabolite, paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and/or vinorelbine; (c) 1-D-ribofuranosyl-1,2,4-triazole-3 carboxamide, 9->2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir; (d) a PD-1 inhibitor or anti-PD-1 antibody such as KEYTRUDA® (pembrolizumab), OPDIVO® (nivolumab), or LIBTAYO (cemiplimab); (e) a PD-L1 inhibitor or anti-PD-L1 antibody such as TECENTRIQ (atezolizumab), IMFINZI (durvalumab), or BAVENCIO (avelumab); or (f) a CTLA-4 inhibitor or anti-CTLA-4 antibody such as YERVOY® ipilimumab. It is predicted that the combination of immune checkpoint inhibition (PD-1 inhibition, PD-L1 inhibition, and/or CTLA-4 inhibition) with NEO-201 may be particularly efficacious for treatment of hematological malignancy. See Vargas et al., Immunity. 2017 Apr. 18; 46(4): 577-586 and Taylor et al., J Clin Invest. 2017; 127(9):3472-3483, each of which is hereby incorporated by reference in its entirety.

Other therapeutic regimens wherein the combination thereof with NEO-201 may elicit synergistic effects in treating conditions wherein MDSCs are involved in pathology include radiation therapies. NEO-201 may increase the efficacy of such other therapeutic agents or therapeutic regimens, particularly in individuals who are or become resistant or recalcitrant to treatment with a particular therapeutic agent or regimen because of MDSC-induced immunosuppression.

Combination of NEO-201 with Cell Therapies

NEO-201 because of its ability to ablate gMDSCs should also improve the efficacy of immune cell therapies, e.g., CAR-T and CAR-NK cell therapies, particularly during use of CAR-T and CAR-NK cells for the treatment of cancer, infection, autoimmune and inflammatory indications.

Use of NEO-201 with CAR-T Cells

Chimeric antigen receptor T cells (also known as CART cells) are T cells that have been genetically engineered to produce an artificial T cell receptor for use in immunotherapy. Chimeric antigen receptors (CARs, also known as chimeric immunoreceptors, chimeric T cell receptors or artificial T cell receptors) are receptor proteins that have been engineered to give T cells the new ability to target a specific protein. The receptors are chimeric because they combine both antigen-binding and T cell activating functions into a single receptor.

In some embodiments CART cell therapy uses T cells engineered with CARs for cancer therapy. The premise of CAR T immunotherapy is to modify T cells to recognize target cells, e.g., cancer cells in order to more effectively target and destroy them. The T cells are harvested, genetically altered and then infused into a patient where the resulting CAR T cells selectively attack or elicit an effect on target cells, e.g., tumor cells, infected cells or autoimmune cells. CAR T cells include CD4⁺ and CD8⁺ T cells, and combinations thereof.

CAR T cells can be either derived from T cells in a patient's own blood (autologous) or derived from the T cells of another healthy donor (allogeneic). Once isolated from a person, these T cells are genetically engineered to express a specific CAR, which programs them to target an antigen that is present on the surface of tumors. After CAR T cells are infused into a patient, they act as a “living drug” against cancer cells. When they come in contact with their targeted antigen on a cell, CAR T cells bind to it and become activated, then proceed to proliferate and become cytotoxic. CAR T cells destroy cells through several mechanisms, including extensive stimulated cell proliferation, increasing the degree to which they are toxic to other living cells (cytotoxicity) and by causing the increased secretion of factors that can affect other cells such as cytokines, interleukins and growth factors.

CAR-T cells are used to treat various blood cancers as well as solid tumors. Also, while most CAR-T cell studies focus on creating a CAR-T cell that can eradicate a certain cell population (for instance, CAR-T cells that target lymphoma cells), there are other potential uses for this technology. T cells can also mediate autoimmune reactions to self-antigens. A regulatory T cell outfitted with a CAR can be used to confer tolerance to a specific antigen, e.g., in organ transplantation or autoimmune or inflammatory diseases like lupus and RA.

A “chimeric receptor” generally refers to a cell-surface receptor comprising an extracellular ligand binding domain, a transmembrane domain and a cytoplasmic co-stimulatory signaling domain in a combination that is not naturally found together on a single protein. This particularly includes receptors wherein the extracellular domain and the cytoplasmic domain are not naturally found together on a single receptor protein. Further, the chimeric receptor is different from the TCR expressed in the native T cell lymphocyte.

As described in U.S. Pat. Nos. 5,359,046, 5,686,281 and 6,103,521, the extracellular domain may be obtained from any of the wide variety of extracellular domains or secreted proteins associated with ligand binding and/or signal transduction. The extracellular domain may be part of a protein which is monomeric, homodimeric, heterodimeric, or associated with a larger number of proteins in a non-covalent complex. In particular, the extracellular domain may consist of an Ig heavy chain which may in turn be covalently associated with Ig light chain by virtue of the presence of CH1 and hinge regions, or may become covalently associated with other Ig heavy/light chain complexes by virtue of the presence of hinge, CH2 and CH3 domains. In the latter case, the heavy/light chain complex that becomes joined to the chimeric construct may constitute an antibody with a specificity distinct from the antibody specificity of the chimeric construct. Depending on the function of the antibody, the desired structure and the signal transduction, the entire chain may be used or a truncated chain may be used, where all or a part of the CH1, CH2, or CH3 domains may be removed or all or part of the hinge region may be removed.

The extracellular domains of CARs are often derived from immunoglobulins and include antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR).

A chimeric receptor may be designed to treat any cancer for which a specific monoclonal antibody exists or is capable of being generated. In particular, cancers such as neuroblastoma, small cell lung cancer, melanoma, ovarian cancer, renal cell carcinoma, colon cancer, Hodgkin's lymphoma, and acute lymphoblastic leukemia (e.g., childhood acute lymphoblastic leukemia) have antigens which may be targeted by such chimeric receptors.

The transmembrane domain may be contributed by the protein contributing the multispecific extracellular inducer clustering domain, the protein contributing the effector function signaling domain, the protein contributing the proliferation signaling portion, or by a totally different protein. For the most part it will be convenient to have the transmembrane domain naturally associated with one of the domains. In some cases it will be desirable to employ the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FCεR1γ chains or related proteins. In some instances, the transmembrane domain will be selected or modified by amino acid substitution to avoid binding of such domains to the transmembrane domains of the same or different surface membrane proteins to minimize interactions with other members of the receptor complex. In other cases it will be desirable to employ the transmembrane domain of ζ, η or FcεR1γ chains and -β, MB1 (Igα), B29 or CD3γ, ζ, or ε, in order to retain physical association with other members of the receptor complex. Examples of suitable transmembrane regions for use with the invention include the constant (Fc) regions of immunoglobins, human CD8a, and artificial linkers that serve to move the targeting moiety away from the cell surface for improved access to and binding on target cells, however any transmembrane region sufficient to anchor the CAR in the membrane can be used. Persons of skill are aware of numerous transmembrane regions and the structural elements (such as lipophilic amino acid regions) that produce transmembrane domains in numerous membrane proteins and therefore can substitute any convenient sequence.

The cytoplasmic domain of the chimeric receptors of the invention can comprise a signaling domain (e.g., co-stimulatory signaling domain) by itself or combined with any other desired cytoplasmic domain(s) useful in the context of this chimeric receptor type, such as for example, a 4-1BB signaling domain, a CD3ζ signaling domain and/or a CD28 signaling domain. The 4-1BB, CD3ζ and CD28 signaling domains are well characterized, including for example, their use in chimeric receptors. In one embodiment, the cytoplasmic domain of the chimeric receptors can comprise the 4-1BB signaling domain by itself or combined with any other desired cytoplasmic domain(s) useful in the context of this chimeric receptor type. In a most preferred embodiment of the invention the extracellular domain comprises a single chain variable domain of a monoclonal antibody, the transmembrane domain comprises the hinge and transmembrane domain of CD8a, and the cytoplasmic domain comprises the signaling domain of CD3ζ and the signaling domain of 4-1BB. The CD8a hinge and transmembrane domain consists of 69 amino acids translated from the 207 nucleotides at positions 815-1021 of GenBank Accession No. NM_001768. The CD3ζ signaling domain of the preferred embodiment contains 112 amino acids translated from 339 nucleotides at positions 1022-1360 of GenBank Accession No. NM_000734.

In adoptive immunotherapy, the patient's circulating lymphocytes, or tumor infiltrated lymphocytes, are isolated in vitro, activated by lymphokines such as IL-2 or transduced with genes for tumor necrosis, and readministered. To achieve this, one would administer to an animal, or human patient, an immunologically effective amount of activated lymphocytes genetically modified to express a tumor-specific chimeric receptor gene as described herein. The activated lymphocytes will most preferably be the patient's own cells that were earlier isolated from a blood or tumor sample and activated and expanded in vitro. The antigen-specific CAR-T cells can be expanded in vitro for use in adoptive cellular immunotherapy in which infusions of such cells have been shown to have anti-tumor reactivity in a tumor-bearing host.

Genetic modification for introduction of the CAR construct into T cells can be accomplished by transducing (or otherwise delivering) a T cell composition with a recombinant DNA or RNA construct, such as for example, a vector. A vector may be any agent capable of delivering or maintaining nucleic acid in a host cell, and includes viral vectors (e.g. retroviral vectors, lentiviral vectors, adenoviral vectors, or adeno-associated viral vectors), plasmids, naked nucleic acids, nucleic acids complexed with polypeptide or other molecules and nucleic acids immobilized onto solid phase particles. The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

Selection of promoter and other regulatory sequences for protein expression are well known to those of skill in the art. Cell specific promoters for expression in T-cells include, but are not limited to, human CD2, distal Lck, and proximal Lck. In other embodiments, non-tissue specific promoters such as non-tissue specific promoters including viral promoters such as cytomegalovirus (CMV) promoter, β actin promoter phosphoglycerate kinase (PGK) promoter, ubiquitin promoter, and EF-1α promoter can be used. This list is not meant to be limiting. An expression construction preferably also includes sequences to allow for the replication of the expression construct. Transcription of the DNA encoding the polypeptides of the present invention by higher eukaryotes may be increased by inserting an enhancer sequence into the vector. Enhancers are cis-acting elements of DNA, usually about from 10 to 300 by that act on a promoter to increase its transcription. Examples including the SV40 enhancer on the late side of the replication origin by 100 to 270, a cytomegalovirus early promoter enhancer, the polyoma enhancer on the late side of the replication origin, and adenovirus enhancers. Preferably, a retroviral vector (either gamma-retroviral or lentiviral) is employed for the introduction of the CAR nucleic acid construct into the cell. For example, a polynucleotide encoding a co-stimulatory ligand protein (e.g., tumor necrosis factor (TNF) ligand, such as 4-1BBL, OX4OL, CD70, LIGHT, and CD30L, or an Ig superfamily ligand, such as CD80 and CD86), or a receptor that binds an antigen, or a variant, or a fragment thereof, can be cloned into a retroviral vector and expression can be driven from its endogenous promoter, from the retroviral long terminal repeat, or from a promoter specific for a target cell type of interest. Non-viral vectors may be used as well.

CAR-T cells are typically expanded and activated in vitro to reach therapeutically sufficient numbers prior to administration to a subject. The cells may be expanded either non-specifically with mitogenic αCD3 and αCD28 antibodies, or through the use of genetically modified antigen-presenting cell lines or particles which display the antigen targeted by the CAR binding domain (and in some cases additional costimulatory molecules). Other methods to selectively propagate T cells to constitutively express CAR include co-expression with transgenes for selection under cytocidal concentrations of drug and/or sorting, such as using magnetic beads that recognize introduced proteins co-expressed with CAR. Antigen-specific expansion is preferred, as CAR-mediated T-cell activation is thought to depend on and to increase with the binding affinity to cognate antigen. In the event that the CAR-T cells of the present invention are non-specifically expanded without activation prior to treatment with a nucleic acid targeting agent, they may be activated in vitro prior to administration to a subject, again using cell lines or particles which display the antigen targeted by the CAR binding domain.

The diseased cell can be from any type of cancer, of any tissue or cell type origin. Suitable target cells include but are not limited to cells of the following malignancies: Leukemia including Chronic Myelogenous Leukemia (CML), Chronic Lymphocytic leukemia (CLL), Acute Myelogenous Leukemia (AML), and Acute Lymphoblastic Leukemia (ALL); Multiple myeloma (MM); Non-Hodgkin lymphoma and Hodgkin's disease (lymphoma); solid tumors, including breast, lung, ovarian and testicular cancers, prostate cancer, colon cancer, melanoma, renal carcinoma cell, neuroblastoma, and head and neck tumors.

CARs and CAR-T-derived effector cells can be designed which target any desired antigen. Examples of target antigens include by way of example: 0772P (CA125, MUC16; GenBank accession no. AF36148); adipophilin (perilipin-2, Adipose differentiation-related protein, ADRP, ADFP, MGC10598; NCBI Reference Sequence: NP-001113.2); AIM-2 (Absent In Melanoma 2, PYHIN4, Interferon-Inducible Protein AIM2; NCBI Reference Sequence: NP-004824.1); ALDH1 A1 (Aldehyde Dehydrogenase 1 Family, Member A1, ALDH1, PUMB 1, Retinaldehyde Dehydrogenase 1, ALDC, ALDH-E1, ALHDII, RALDH 1, EC 1.2.1.36, ALDH11, HEL-9, HEL-S-53e, HEL12, RALDH1, Acetaldehyde Dehydrogenase 1, Aldehyde Dehydrogenase 1, Soluble, Aldehyde Dehydrogenase, Liver Cytosolic, ALDH Class 1, Epididymis Luminal Protein 12, Epididymis Luminal Protein 9, Epididymis Secretory Sperm Binding Protein Li 53e, Retinal Dehydrogenase 1, RaIDH1, Aldehyde Dehydrogenase Family 1 Member A1, Aldehyde Dehydrogenase, Cytosolic, EC 1.2.1; NCBI Reference Sequence: NP-000680.2); alpha-actinin-4 (ACTN4, Actinin, Alpha 4, FSGS1, Focal Segmental Glomerulosclerosis 1, Non-Muscle Alpha-Actinin 4, F-Actin Cross-Linking Protein, FSGS, ACTININ-4, Actinin Alpha4 Isoform, alpha-actinin-4; NCBI Reference Sequence: NP-004915.2); alpha-fetoprotein (AFP, HPAFP, FETA, alpha-1-fetoprotein, alpha-fetoglobulin, Alpha-1-fetoprotein, Alpha-fetoglobulin, HP; GenBank: AAB58754.1); Amphiregulin (AREG, SDGF, Schwannoma-Derived Growth Factor, Colorectum Cell-Derived Growth Factor, AR, CRDGF; GenBank: AAA51781.1); ARTC1 (ART1, ADP-Ribosyltransferase 1, Mono(ADP-Ribosyl)Transferase 1, ADP-Ribosyltransferase C2 And C3 Toxin-Like 1, ART2, CD296, RT6, ADP-Ribosyltransferase 2, GPI-Linked NAD(P)(+)-Arginine ADP-Ribosyltransferase 1, EC 2.4.2.31, CD296 Antigen; NP); ASLG659; ASPHDI (Aspartate Beta-Hydroxylase Domain Containing 1, Aspartate Beta-Hydroxylase Domain-Containing Protein 1, EC 1.14.11., GenBank: AA144153.1); B7-H4 (VTCN1, V-Set Domain Containing T Cell Activation Inhibitor 1, B7H4, B7 Superfamily Member 1, Immune Costimulatory Protein B7-H4, B7h.5, T-Cell Costimulatory Molecule B7x, B7S1, B7X, VCTN1, H4, B7 Family Member, PRO1291, B7 Family Member, H4, T Cell Costimulatory Molecule B7x, V-Set Domain-Containing T-Cell Activation Inhibitor 1, Protein B7S1; GenBank: AAZ 17406.1); BAFF-R (TNFRSF13C, Tumor Necrosis Factor Receptor Superfamily, Member 13C, BAFFR, B-Cell-Activating Factor Receptor, BAFF Receptor, BLyS Receptor 3, CVID4, BROMIX, CD268, B Cell-Activating Factor Receptor, prolixin, Tumor Necrosis Factor Receptor Superfamily Member 13C, BR3, CD268 Antigen; NCBI Reference Sequence: NP-443177.1); BAGE-1; BCLX (L); BCR-ABL fusion protein (b3a2); beta-catenin (CTNNB1, Catenin (Cadherin-Associated Protein), Beta 1, 88 kDa, CTNNB, MRD19, Catenin (Cadherin-Associated Protein), Beta 1 (88 kD), armadillo, Catenin Beta-1; GenBank: CAA61107.1); BING-4 (WDR46, WD Repeat Domain 46, C6orf11, BING4, WD Repeat-Containing Protein BING4, Chromosome 6 Open Reading Frame 11, FP221, UTP7, WD Repeat-Containing Protein 46; NP); BMPR1 B (bone morphogenetic protein receptor-type IB, GenBank accession no. NM-00120; NP); B-RAF (Brevican (BCAN, BEHAB, GenBank accession no. AF22905); Brevican (BCAN, Chondroitin Sulfate Proteoglycan 7, Brain-Enriched Hyaluronan-Binding Protein, BEHAB, CSPG7, Brevican Proteoglycan, Brevican Core Protein, Chondroitin Sulfate Proteoglycan BEHAB; GenBank: AAH27971.1); CALCA (Calcitonin-Related Polypeptide Alpha, CALC1, Calcitonin 1, calcitonin, Alpha-Type CGRP, Calcitonin Gene-Related Peptide I, CGRP-I, CGRP, CGRP1, CT, KC, Calcitonin/Calcitonin-Related Polypeptide, Alpha, katacalcin; NP); CASP-5 (CASP5, Caspase 5, Apoptosis-Related Cysteine Peptidase, Caspase 5, Apoptosis-Related Cysteine Protease, Protease ICH-3, Protease TY, ICE(rel)-111, ICE(rel)III, ICEREL-III, ICH-3, caspase-5, TY Protease, EC 3.4.22.58, ICH3, EC 3.4.22; NP); CASP-8; CD19 (CD19-B-lymphocyte antigen CD19 isoform 2 precursor, B4, CVID3 [Homo sapiens], NCBI Reference Sequence: NP-001761.3); CD20 (CD20-B-lymphocyte antigen CD20, membrane-spanning 4-domains, subfamily A, member 1, B1,Bp35,CD20,CVID5,LEU-16,MS4A2,S7; NCBI Reference Sequence: NP-690605.1); CD21 (CD21 (CR2 (Complement receptor or C3DR (C3d/Epstein Barr virus receptor) or Hs.73792 GenBank accession no. M2600); (CD22 (B-cell receptor CD22-B isoform, BL-CAM, Lyb-8, LybB, SIGLEC-2, FLJ22814, GenBank accession No. AK02646); CD22; CD33 (CD33 Molecule, CD33 Antigen (Gp67), Sialic Acid Binding Ig-Like Lectin 3, Sialic Acid-Binding Ig-Like Lectin 3, SIGLEC3, gp67, SIGLEC-3, Myeloid Cell Surface Antigen CD33, p67, Siglec-3, CD33 Antigen; GenBank: AAH28152.1); CD45; CD70 (CD70-tumor necrosis factor (ligand) superfamily, member 7; surface antigen CD70; Ki-24 antigen; CD27 ligand; CD27-L; tumor necrosis factor ligand superfamily member 7; NCBI Reference Sequence for species Homo sapiens: NP-001243.1); CD72 (CD72 (B-cell differentiation antigen CD72, Lyb-; 359 aa, μl: 8.66, MW: 40225, TM: 1 [P] Gene Chromosome: 9p13.3, GenBank accession No. NP-001773.); CD79a (CD79a (CD79A, CD79a, immunoglobulin-associated alpha; CD79b (CD79b (CD79B, CD79b, IGb (immunoglobulin-associated beta), B29, GenBank accession no. NM-000626 or 1103867); Cdc27 (Cell Division Cycle 27, DOS1430E, D17S978E, Anaphase Promoting Complex Subunit 3, Anaphase-Promoting Complex Subunit 3, ANAPC3, APC3, CDC27Hs, H-NUC, CDC27 Homolog, Cell Division Cycle 27 Homolog (S. cerevisiae), HNUC, NUC2, Anaphase-Promoting Complex, Protein 3, Cell Division Cycle 27 Homolog, Cell Division Cycle Protein 27 Homolog, Nuc2 Homolog; GenBank: AAH11656.1); CDK4 (Cyclin-Dependent Kinase 4, Cell Division Protein Kinase 4, PSK-J3, EC 2.7.11.22, CMM3, EC 2.7.11; NCBI Reference Sequence: NP-000066.1); CDKN2A (Cyclin-Dependent Kinase Inhibitor 2A, MLM, CDKN2, MTS1, Cyclin-Dependent Kinase Inhibitor 2A (Melanoma, P16, Inhibits CDK4), Cyclin-Dependent Kinase 4 Inhibitor A, Multiple Tumor Suppressor 1, CDK4I, MTS-1, CMM2, P16, ARF, INK4, INK4A, P14, P14ARF, P16-INK4A, P16INK4, P16INK4A, P19, P19ARF, TP16, CDK4 Inhibitor P16-INK4, Cell Cycle Negative Regulator Beta, p14ARF, p16-INK4, p16-INK4a, p16INK4A, p19ARF; NP); CEA; CLL1 (CLL-1; CLPP (Caseinolytic Mitochondrial Matrix Peptidase Proteolytic Subunit, Endopeptidase Clp, EC 3.4.21.92, PRLTS3, ATP-Dependent Protease ClpAP (E. coli), ClpP (Caseinolytic Protease, ATP-Dependent, Proteolytic Subunit, E. coli) Homolog, ClpP Caseinolytic Peptidase, ATP-Dependent, Proteolytic Subunit Homolog (E. coli), ClpP Caseinolytic Protease, ATP-Dependent, Proteolytic Subunit Homolog (E. coli), human, Proteolytic Subunit, ATP-Dependent Protease ClpAP, Proteolytic Subunit, Human, ClpP Caseinolytic Peptidase ATP-Dependent, Proteolytic Subunit, ClpP Caseinolytic Peptidase, ATP-Dependent, Proteolytic Subunit Homolog, ClpP Caseinolytic Protease, ATP-Dependent, Proteolytic Subunit Homolog, Putative ATP-Dependent Clp Protease Proteolytic Subunit, Mitochondrial; NP); COA-1; CPSF; CRIPTO (CRIPTO (CR, CR1, CRGF, CRIPTO, TDGF 1, teratocarcinoma-derived growth factor, GenBank accession no. NP-003203 or NM-00321); Cw6; CXCR5; CXORF61 CXORF61-chromosome X open reading frame 61[Homo sapiens], NCBI Reference Sequence: NP-001017978.1); cyclin Di (CCND1, BCL1, PRAD1, D11S287E, B-Cell CLL/Lymphoma 1, B-Cell Lymphoma 1 Protein, BCL-1 Oncogene, PRAD1 Oncogene, Cyclin DI (PRAD1: Parathyroid Adenomatosis 1), G1/S-Specific Cyclin DI, Parathyroid Adenomatosis 1, U21B31, G1/S-Specific Cyclin-D1, BCL-1; NCBI Reference Sequence: NP-444284.1); Cyclin-A1 (CCNA1, CT146, Cyclin A1; GenBank: AAH36346.1); dek-can fusion protein; DKK1 (Dickkopf WNT Signaling Pathway Inhibitor 1, SK, hDkk-1, Dickkopf (Xenopus laevis) Homolog 1, Dickkopf 1 Homolog (Xenopus laevis), DKK-1, Dickkopf 1 Homolog, Dickkopf Related Protein-1, Dickkopf-1 Like, Dickkopf-Like Protein 1, Dickkopf-Related Protein 1, Dickkopf-1, Dkk-1; GenBank: AAQ89364.1); DR1 (Down-Regulator Of Transcription 1, TBP-Binding (Negative Cofactor 2), Negative Cofactor 2-Beta, TATA-Binding Protein-Associated Phosphoprotein, NC2, NC2-BETA, Protein Drl, NC2-beta, Down-Regulator Of Transcription 1; NCBI Reference Sequence: NP-001929.1); DR13 (Major Histocompatibility Complex, Class II, DR Beta 1, HLA-DR1B, DRw10, DW2.2/DR2.2, SS1, DRB1, HLA-DRB, HLA Class II Histocompatibility Antigen, DR-1 Beta Chain, Human Leucocyte Antigen DRB1, Lymphocyte Antigen DRB1, MHC Class II Antigen, MHC Class II HLA-DR Beta 1 Chain, MHC Class II HLA-DR-Beta Cell Surface Glycoprotein, MHC Class II HLA-DRw10-Beta, DR-1, DR-12, DR-13, DR-14, DR-16, DR-4, DR-5, DR-7, DR-8, DR-9, DR1, DR12, DR13, DR14, DR16, DR4, DR5, DR7, DRB, DR9, DRw11, DRw8, HLA-DRB2, Clone P2-Beta-3, MHC Class II Antigen DRB1*1, MHC Class II Antigen DRB1*10, MHC Class II Antigen DRB1*11, MHC Class II Antigen DRB1*12, MHC Class II Antigen DRB1*13, MHC Class II Antigen DRB1*14, MHC Class II Antigen DRB1*15, MHC Class II Antigen DRB1*16, MHC Class II Antigen DRB1*3, MHC Class II Antigen DRB1*4, MHC Class II Antigen DRB1*7, MHC Class II Antigen DRB1*8, MHC Class II Antigen DRB1*9; NP); E16 (E16 (LAT1, SLC7A5, GenBank accession no. NM-00348); EDAR (EDAR—tumor necrosis factor receptor superfamily member EDAR precursor, EDA-A1 receptor; downless homolog; ectodysplasin-A receptor; ectodermal dysplasia receptor; anhidrotic ectodysplasin receptor 1, DL; ECTD10A; ECTD10B; ED1R; ED3; ED5; EDA-AIR; EDA1R; EDA3; HRM1 [Homo sapiens]; NCBI Reference Sequence: NP-071731.1); EFTUD2 (Elongation FactorTu GTP Binding Domain Containing 2, Elongation FactorTu GTP-Binding Domain-Containing Protein 2, hSNU114, SNU114 Homolog, U5 SnRNP-Specific Protein, 116 KDa, MFDGA, KIAA0031, 116 KD, U5 SnRNP Specific Protein, 116 KDa U5 Small Nuclear Ribonucleoprotein Component, MFDM, SNRNP116, Snrp116, Snull 14, U5-116KD, SNRP116, U5-116 KDa; GenBank: AAH02360.1); EGFR (Epidermal Growth Factor Receptor, ERBB, Proto-Oncogene C-ErbB-1, Receptor Tyrosine-Protein Kinase ErbB-1, ERBB 1, HER1, EC 2.7.10.1, Epidermal Growth Factor Receptor (Avian Erythroblastic Leukemia Viral (V-Erb-B) Oncogene Homolog), Erythroblastic Leukemia Viral (V-Erb-B) Oncogene Homolog (Avian), P1G61, Avian Erythroblastic Leukemia Viral (V-Erb-B) Oncogene Homolog, Cell Growth Inhibiting Protein 40, Cell Proliferation-Inducing Protein 61, mENA, EC 2.7.10; GenBank: AAH94761.1); EGFR-G719A; EGFR-G719C; EGFR-G719S; EGFR-L858R; EGFR-L861 Q; EGFR-57681; EGFR-T790M; Elongation factor 2 (EEF2, Eukaryotic Translation Elongation Factor 2, EF2, Polypeptidyl-TRNA Translocase, EF-2, SCA26, EEF-2; NCBI Reference Sequence: NP-001952.1); ENAH (hMena) (Enabled Homolog (Drosophila), MENA, Mammalian Enabled, ENA, NDPP1, Protein Enabled Homolog; GenBank: AAH95481.1)-results for just “ENAH” not “ENAH (hMena)”; EpCAM (Epithelial Cell Adhesion Molecule, M4S1, MIC 18, Tumor-Associated Calcium Signal Transducer 1, TACSTD1, TROP1, Adenocarcinoma-Associated Antigen, Cell Surface Glycoprotein Trop-1, Epithelial Glycoprotein 314, Major Gastrointestinal Tumor-Associated Protein GA733-2, EGP314, KSA, DIAR5, HNPCC8, Antigen Identified By Monoclonal Antibody AUA1, EGP-2, EGP40, ESA, KS 1/4, MK-1, Human Epithelial Glycoprotein-2, Membrane Component, Chromosome 4, Surface Marker (35 kD Glycoprotein), EGP, Ep-CAM, GA733-2, M1S2, CD326 Antigen, Epithelial Cell Surface Antigen, hEGP314, KS 1/4 Antigen, ACSTD1; GenBank: AAH14785.1); EphA3 (EPH Receptor A3, ETK1, ETK, TYRO4, HEK, Eph-Like Tyrosine Kinase 1, Tyrosine-Protein Kinase Receptor ETK1, EK4, EPH-Like Kinase 4, EC 2.7.10.1, EPHA3, HEK4, Ephrin Type-A Receptor 3, Human Embryo Kinase 1,TYRO4 Protein Tyrosine Kinase, hEK4, Human Embryo Kinase, Tyrosine-Protein Kinase TYRO4, EC 2.7.10; GenBank: AAH63282.1); EphB2R; Epiregulin (EREG, ER, proepiregulin; GenBank: AA136405.1); ETBR (EDNRB, Endothelin Receptor Type B, HSCR2, HSCR, Endothelin Receptor Non-Selective Type, ET-B, ET-BR, ETRB, ABCDS, WS4A, ETB, Endothelin B Receptor; NP); ETV6-AML1 fusion protein; EZH2 (Enhancer Of Zeste Homolog 2 (Drosophila), Lysine N-Methyltransferase 6, ENX-1, KMT6 EC 2.1.1.43, EZH1, WVS, Enhancer Of Zeste (Drosophila) Homolog 2, ENX1, EZH2b, KMT6A, WVS2, Histone-Lysine N-Methyltransferase EZH2, Enhancer Of Zeste Homolog 2, EC 2.1.1; GenBank: AAH10858.1); FcRH1 (FCRL1, Fc Receptor-Like 1, FCRH1, Fc Receptor Homolog 1, FcR-Like Protein 1, Immune Receptor Translocation-Associated Protein 5, IFGP1, IRTA5, hIFGP1, IFGP Family Protein 1, CD307a, Fc Receptor-Like Protein 1, Immunoglobulin Superfamily Fc Receptor, Gp42, FcRL1, CD307a Antigen; GenBank: AAH33690.1); FcRH2 (FCRL2, Fc Receptor-Like 2, SPAP1, SH2 Domain-Containing Phosphatase Anchor Protein 1, Fc Receptor Homolog 2, FcR-Like Protein 2, Immunoglobulin Receptor Translocation-Associated Protein 4, FCRH2, IFGP4, IRTA4, IFGP Family Protein 4, SPAP1A, SPAP1 B, SPAP1C, CD307b, Fc Receptor-Like Protein 2, Immune Receptor Translocation-Associated Protein 4, Immunoglobulin Superfamily Fc Receptor, Gp42, SH2 Domain Containing Phosphatase Anchor Protein 1, FcRL2, CD307b Antigen; GenBank: AAQ88497.1); FcRH5 (FCRL5, Fc Receptor-Like 5, IRTA2, Fc Receptor Homolog 5, FcR-Like Protein 5, Immune Receptor Translocation-Associated Protein 2, BXMAS1, FCRH5, CD307, CD307e, PRO820, Fc Receptor-Like Protein 5, Immunoglobulin Superfamily Receptor Translocation Associated 2 (IRTA2), FCRL5, CD307e Antigen; GenBank: AAI01070.1); FLT3-ITD; FN1 (Fibronectin 1, Cold-Insoluble Globulin, FN, Migration-Stimulating Factor, CIG, FNZ, GFND2, LETS, ED-B, FINC, GFND, MSF, fibronectin; GenBank: AA143764.1); G250 (MN, CAIX, Carbonic Anhydrase IX, Carbonic Dehydratase, RCC-Associated Protein G250, Carbonate Dehydratase IX, Membrane Antigen MN, Renal Cell Carcinoma-Associated Antigen G250, CA-IX, P54/58N, pMW1, RCC-Associated Antigen G250, Carbonic Anhydrase 9; NP); —alias results for “G250” not “G250/MN/CAIX”; GAGE-1,2,8; GAGE-3,4,5,6,7; GDNF-Ral (GDNF family receptor alpha 1; GFRA1; GDNFR; GDNFRA; RETL1; TRNR1; RET1 L; GDNFR-alphal; GFR-ALPHA-; U95847; BC014962; NM-145793 NM-005264); GEDA (GenBank accession No. AY26076); GFRA1-GDNF family receptor alpha-1; GDNF receptor alpha-1; GDNFR-alpha-1; GFR-alpha-1; RET ligand 1; TGF-beta-related neurotrophic factor receptor 1 [Homo sapiens]; ProtKB/Swiss-Prot: P56159.2; glypican-3 (GPC3, Glypican 3, SDYS, Glypican Proteoglycan 3, Intestinal Protein OCI-5, GTR2-2, MXR7, SGBS1, DGSX, OCI-5. SGB, SGBS, Heparan Sulphate Proteoglycan, Secreted Glypican-3, OC15; GenBank: AAH35972.1); GnTVf; gp100 (PMEL, Premelanosome Protein, SILV, D12S53E, PMEL17, SIL, Melanocyte Protein Pmel 17, Melanocytes Lineage-Specific Antigen GP100, Melanoma-Associated ME20 Antigen, Silver Locus Protein Homolog, ME20-M, ME20M, P1, P100, Silver (Mouse Homolog) Like, Silver Homolog (Mouse), ME20, SI, Melanocyte Protein Mel 17, Melanocyte Protein PMEL, Melanosomal Matrix Proteinl7, Silver, Mouse, Homolog Of; GenBank: AAC60634.1); GPC; GPNMB (Glycoprotein (Transmembrane) Nmb, Glycoprotein NMB, Glycoprotein Nmb-Like Protein, osteoactivin, Transmembrane Glycoprotein HGFIN, HGFIN, NMB, Transmembrane Glycoprotein, Transmembrane Glycoprotein NMB; GenBank: AAH32783.1); GPR172A (G protein-coupled receptor 172A; GPCR41; FLJ11856; D15Ertd747e); NP-078807.1; NM-024531.3); GPR19 (G protein-coupled receptor 19; Mm.478; NP-006134.1; NM-006143.2); GPR54 (KISS1 receptor; KISSiR; GPR54; HOT7T175; AXOR1; NP-115940.2; NM-032551.4); HAVCR1 (Hepatitis A Virus Cellular Receptor 1, T-Cell Immunoglobulin Mucin Family Member 1, Kidney Injury Molecule 1, KIM-1, KIM1, TIM, TIM-1, TIM1, TIMD-1, TIMD1, T-Cell Immunoglobulin Mucin Receptor 1, T-Cell Membrane Protein 1, HAVCR, HAVCR-1, T Cell Immunoglobin Domain And Mucin Domain Protein 1, HAVcr-1, T-Cell Immunoglobulin And Mucin Domain-Containing Protein 1; GenBank: AAH13325.1); HER2 (ERBB2, V-Erb-B2 Avian Erythroblastic Leukemia Viral Oncogene Homolog 2, NGL, NEU, Neuro/Glioblastoma Derived Oncogene Homolog, Metastatic Lymph Node Gene 19 Protein, Proto-Oncogene C-ErbB-2, Proto-Oncogene Neu, Tyrosine Kinase-Type Cell Surface Receptor HER2, MLN 19, p185erbB2, EC 2.7.10.1, V-Erb-B2 Avian Erythroblastic Leukemia Viral Oncogene Homolog 2 (Neuro/Glioblastoma Derived Oncogene Homolog), CD340, HER-2, HER-2/neu, TKR1, C-Erb B2/Neu Protein, herstatin, Neuroblastoma/Glioblastoma Derived Oncogene Homolog, Receptor Tyrosine-Protein Kinase ErbB-2, V-Erb-B2 Erythroblastic Leukemia Viral Oncogene Homolog 2, Neuro/Glioblastoma Derived Oncogene Homolog, MLN19, CD340 Antigen, EC 2.7.10; NP); HER-2/neu-alias of above; HERV-K-MEL; HLA-DOB (Beta subunit of MHC class II molecule (Ia antigen) that binds peptides and presents them to CD4+ T lymphocytes); 273 aa, al: 6.56, MW: 30820.TM: 1 [P] Gene Chromosome: 6p21.3, GenBank accession No. NP-002111); hsp70-2 (HSPA2, Heat Shock 70 kDa Protein 2, Heat Shock 70 kD Protein 2, HSP70-3, Heat Shock-Related 70 KDa Protein 2, Heat Shock 70 KDa Protein 2; GenBank: AAD21815.1); IDO1 (Indoleamine 2,3-Dioxygenase 1, IDO, INDO, Indoleamine-Pyrrole 2,3-Dioxygenase, IDO-1, Indoleamine-Pyrrole 2,3 Dioxygenase, Indolamine 2,3 Dioxygenase, Indole 2,3 Dioxygenase, EC 1.13.11.52; NCBI Reference Sequence: NP-002155.1); IGF2B3; IL13Ralpha2 (IL13RA2, Interleukin 13 Receptor, Alpha 2, Cancer/Testis Antigen 19, Interleukin-13-Binding Protein, IL-13R-alpha-2, IL-13RA2, IL-13 Receptor Subunit Alpha-2, IL-13R Subunit Alpha-2, CD213A2, CT19, IL-13R, IL13BP, Interleukin 13 Binding Protein, Interleukin 13 Receptor Alpha 2 Chain, Interleukin-13 Receptor Subunit Alpha-2, IL13R, CD213a2 Antigen; NP); IL20Rα; Intestinal carboxyl esterase; IRTA2 (alias of FcRH5); Kallikrein 4 (KLK4, Kallikrein-Related Peptidase 4, PRSS17, EMSP1, Enamel Matrix Serine Proteinase 1, Kallikrein-Like Protein 1, Serine Protease 17, KLK-L1, PSTS, A12A1, Kallikrein 4 (Prostase, Enamel Matrix, Prostate), ARM1, EMSP, Androgen-Regulated Message 1, Enamel Matrix Serine Protease 1, kallikrein, kallikrein-4, prostase, EC 3.4.21.-, Prostase, EC 3.4.21; GenBank: AAX30051.1); KIF20A (Kinesin Family Member 20A, RAB6KIFL, RAB6 Interacting, Kinesin-Like (Rabkinesin6), Mitotic a; LAGE-1; LDLR-fucosyltransferase AS fusion protein; Lengsin (LGSN, Lengsin, Lens Protein With Glutamine Synthetase Domain, GLULD1, Glutamate-Ammonia Ligase Domain-Containing Protein 1, LGS, Glutamate-Ammonia Ligase (Glutamine Synthetase) Domain Containing 1, Glutamate-Ammonia Ligase (Glutamine Synthase) Domain Containing 1, Lens Glutamine Synthase-Like; GenBank: AAF61255.1); LGR5 (leucine-rich repeat-containing G protein-coupled receptor 5; GPR49, GPR6; NP-003658.1; NM-003667.2; LY64 (Lymphocyte antigen 64); Ly6E (lymphocyte antigen 6 complex, locus E; Ly67, RIG-E,SCA-2, TSA-; NP-002337.1; NM-002346.2); Ly6G6D (lymphocyte antigen 6 complex, locus G6D; Ly6-D, MEGT; NP-067079.2; NM-021246.2); LY6K (lymphocyte antigen 6 complex, locus K; LY6K; HSJ001348; FLJ3522; NP-059997.3; NM-017527.3); LyPD1-LY6/PLAUR domain containing 1, PHTS [Homo sapiens], GenBank: AAH17318.1); MAGE-A1 (Melanoma Antigen Family A, 1 (Directs Expression Of Antigen MZ2-E, MAGE1, Melanoma Antigen Family A 1, MAGEA1, Melanoma Antigen MAGE-1, Melanoma-Associated Antigen 1, Melanoma-Associated Antigen MZ2-E, Antigen MZ2-E, Cancer/Testis Antigen 1.1, CT1.1, MAGE-1 Antigen, Cancer/Testis Antigen Family 1, Member 1, Cancer/Testis Antigen Family 1, Member 1, MAGE1A; NCBI Reference Sequence: NP-004979.3); MAGE-A10 (MAGEA10, Melanoma Antigen Family A, 10, MAGE10, MAGE-10 Antigen, Melanoma-Associated Antigen 10, Cancer/Testis Antigen 1.10, CT1.10, Cancer/Testis Antigen Family 1, Member 10, Cancer/Testis Antigen Family 1, Member 10; NCBI Reference Sequence: NP-001238757.1); MAGE-A12 (MAGEA12, Melanoma Antigen Family A, 12, MAGE12, Cancer/Testis Antigen 1.12, CT1.12, MAGE12F Antigen, Cancer/Testis Antigen Family 1, Member 12, Cancer/Testis Antigen Family 1, Member 12, Melanoma-Associated Antigen 12, MAGE-12 Antigen; NCBI Reference Sequence: NP-001159859.1); MAGE-A2 (MAGEA2, Melanoma Antigen Family A, 2, MAGE2, Cancer/Testis Antigen 1.2, CT1.2, MAGEA2A, MAGE-2 Antigen, Cancer/Testis Antigen Family 1, Member 2, Cancer/Testis Antigen Family 1, Member 2, Melanoma Antigen 2, Melanoma-Associated Antigen 2; NCBI Reference Sequence: NP-001269434.1); MAGE-A3 (MAGEA3, Melanoma Antigen Family A, 3, MAGE3, MAGE-3 Antigen, Antigen MZ2-D, Melanoma-Associated Antigen 3, Cancer/Testis Antigen 1.3, CT1.3, Cancer/Testis Antigen Family 1, Member 3, HIPS, HYPD, MAGEA6, Cancer/Testis Antigen Family 1, Member 3; NCBI Reference Sequence: NP-005353.1); MAGE-A4 (MAGEA4, Melanoma Antigen Family A, 4, MAGE4, Melanoma-Associated Antigen 4, Cancer/Testis Antigen 1.4, CT1.4, MAGE-4 Antigen, MAGE-41 Antigen, MAGE-X2 Antigen, MAGE4A, MAGE4B, Cancer/Testis Antigen Family 1, Member 4, MAGE-41, MAGE-X2, Cancer/Testis Antigen Family 1, Member 4; NCBI Reference Sequence: NP-001011550.1); MAGE-A6 (MAGEA6, Melanoma Antigen Family A, 6, MAGE6, MAGE-6 Antigen, Melanoma-Associated Antigen 6, Cancer/Testis Antigen 1.6, CT1.6, MAGE3B Antigen, Cancer/Testis Antigen Family 1, Melanoma Antigen Family A 6, Member 6, MAGE-3b, MAGE3B, Cancer/Testis Antigen Family 1, Member 6; NCBI Reference Sequence: NP-787064.1); MAGE-A9 (MAGEA9, Melanoma Antigen Family A, 9, MAGE9, MAGE-9 Antigen, Melanoma-Associated Antigen 9, Cancer/Testis Antigen 1.9, CT1.9, Cancer/Testis Antigen Family 1, Member 9, Cancer/Testis Antigen Family 1, Member 9, MAGEA9A; NCBI Reference Sequence: NP-005356.1); MAGE-C1 (MAGEC1, Melanoma Antigen Family C, 1, Cancer/Testis Antigen 7.1, CT7.1, MAGE-C1 Antigen, Cancer/Testis Antigen Family 7, Member 1, CT7, Cancer/Testis Antigen Family 7, Member 1, Melanoma-Associated Antigen C1; NCBI Reference Sequence: NP-005453.2); MAGE-C2 (MAGEC2, Melanoma Antigen Family C, 2, MAGEE1, Cancer/Testis Antigen 10, CT10, HCA587, Melanoma Antigen, Family E, 1, Cancer/Testis Specific, Hepatocellular Carcinoma-Associated Antigen 587, MAGE-C2 Antigen, MAGE-E1 Antigen, Hepatocellular Cancer Antigen 587, Melanoma-Associated Antigen C2; NCBI Reference Sequence: NP-057333.1); mammaglobin-A (SCGB2A2, Secretoglobin, Family 2A, Member 2, MGB1, Mammaglobin 1, UGB2, Mammaglobin A, mammaglobin-A, Mammaglobin-1, Secretoglobin Family 2A Member 2; NP); MART2 (H HAT, Hedgehog Acyltransferase, SKI1, Melanoma Antigen Recognized By T-Cells 2, Skinny Hedgehog Protein 1, Skn, Melanoma Antigen Recognized ByT Cells 2, Protein-Cysteine N-Palmitoyltransferase HHAT, EC 2.3.1.-; GenBank: AAH39071.1); M-CSF (CSF1, Colony Stimulating Factor 1 (Macrophage), MCSF, CSF-1, lanimostim, Macrophage Colony-Stimulating Factor 1, Lanimostim; GenBank: AAH21117.1); MCSP (SMCP, Sperm Mitochondria-Associated Cysteine-Rich Protein, MCS, Mitochondrial Capsule Selenoprotein, HSMCSGEN1, Sperm Mitochondrial-Associated Cysteine-Rich Protein; NCBI Reference Sequence: NP-109588.2); XAGE-Ib/GAGED2a; WT1 (Wilms Tumor 1, WAGR, GUD, WIT-2, WT33, Amino-Terminal Domain Of EWS, NPHS4, Last Three Zinc Fingers Of The DNA-Binding Domain Of WT 1, AWT 1, Wilms Tumor Protein, EWS-WT1; GenBank: AAB33443.1); VEGF; Tyrosinase (TYR; OCAIA; OCA1A; tyrosinase; SHEP; NP-000363.1; NM-000372.4; GenBank: AAB60319.1); TrpM4 (BR22450, FL20041, TRPM4, TRPM4B, transient receptor potential cation channel, subfamily M, member 4, GenBank accession no. NM-01763); TRP2-INT2; TRP-2; TRP-1/gp75 (Tyrosinase-Related Protein 1, 5,6-Dihydroxyindole-2-Carboxylic Acid Oxidase, CAS2, CATB, TYRP, OCAS, Catalase B, b-PROTEIN, Glycoprotein 75, EC 1.14.18., Melanoma Antigen Gp75, TYRP1, TRP, TYRRP, TRP1, SHEP11, DHICA Oxidase, EC 1.14.18, GP75, EC 1.14.18.1; Triosephosphate isomerase (Triosephosphate isomerase 1, TPID, TriosePhosphate Isomerase, HEL-S-49, TIM, Epididymis Secretory Protein Li 49, TPI, Triosephosphate Isomerase, EC 5.3.1.1; TRAG-3 (CSAG Family Member 2, Cancer/Testis Antigen Family 24, CSAG3B, Member 2, CSAG Family Member 3B, Cancer/Testis Antigen Family 24 Member 2, Cancer/Testis Antigen 24.2, Chondrosarcoma-Associated Gene 2/3 Protein, Taxol-Resistant-Associated Gene 3 Protein, Chondrosarcoma-Associated Gene 2/3 Protein-Like, CT24.2, Taxol Resistance Associated Gene 3, TRAG-3, CSAG3A, TRAG3;); TMEM46 (shisa homolog 2 (Xenopus laevis); SHISA; NP-001007539.1; NM-001007538.1; TMEM118 (ring finger protein, transmembrane2; RNFT2; FU1462; NP-001103373.1; NM-001109903.1; TMEFF1 (transmembrane protein with EGF-like and two follistatin-like domains 1; Tomoregulin-; H7365; C9orf2; C90RF2; U19878; X83961; NM-080655; NM-003692; TGF-betaRII (TGFBR2, Transforming Growth Factor, Beta Receptor II (70/80 kDa), TGFbeta-RII, MFS2, tbetaR-II, TGFR-2, TGF-Beta Receptor Type IIB, TGF-Beta Type II Receptor, TGF-Beta Receptor Type-2, EC 2.7.11.30, Transforming Growth Factor Beta Receptor Type IIC, AAT3, TbetaR-II, Transforming Growth Factor, Beta Receptor II (70-80 kD), TGF-Beta Receptor Type II, FAA3, Transforming Growth Factor-Beta Receptor Type 11, LDS1 B, HNPCC6, LDS2B, LDS2, RITC, EC 2.7.11, TAAD2; TENB2 (TMEFF2, tomoregulin, TPEF, HPP1, TR, putative transmembrane proteoglycan, related to the EGF/heregulin family of growth factors and follistatin); 374 aa, NCBI Accession: AAD55776, AAF91397, AAG49451, NCBI RefSeq: NP-057276; NCBI Gene: 23671; OMIM: 605734; SwissProt Q9UIK5; GenBank accession No. AF179274; AY358907, CAF85723, CQ782436; TAG-2; TAG-1 (Contactin 2 (Axonal), TAG-1, AXT, Axonin-1 Cell Adhesion Molecule, TAX, Contactin 2 (transiently Expressed), TAXI, Contactin-2, Axonal Glycoprotein TAG-1, Transiently-Expressed Axonal Glycoprotein, Transient Axonal Glycoprotein, Axonin-1, TAX-1, TAG1, FAMES; PRF: 444868); SYT-SSX1 or SSX2 fusion protein; survivin; STEAP2 (HGNC 8639, IPCA-1, PCANAP1, STAMPI, STEAP2, STMP, prostate cancer associated gene 1, prostate cancer associated protein 1, six transmembrane epithelial antigen of prostate 2, six transmembrane prostate protein, GenBank accession no. AF45513; STEAP 1 (six transmembrane epithelial antigen of prostate, GenBank accession no. NM-01244; SSX-4; SSX-2 (SSX2, Synovial Sarcoma, X Breakpoint2, X Breakpoint 2, SSX, X Breakpoint 2B, Cancer/Testis Antigen 5.2, X-Chromosome-Related 2, Tumor Antigen HOM-MEL-40, CT5.2, HD21, Cancer/Testis Antigen Family 5, HOM-MEL-40, Isoform B, Cancer/Testis Antigen Family 5 member 2a, member 2a, Protein SSX2, Sarcoma, Sarcoma, Synovial, X-Chromosome-Related 2, synovial, Synovial Sarcoma, X Breakpoint 2B, Synovial Sarcomam, SSX2A; Sp17; SOX10 (SRY (Sex Determining Region Y)-Box 10, mouse, PCWH, DOM, WS4, WS2E, WS4C, Dominant Megacolon, mouse, Human Homolog Of, Dominant Megacolon, SRY-Related HMG-Box Gene 10, Human Homolog Of, transcription Factor SOX-10; GenBank: CAG30470.1); SNRPD1 (Small Nuclear Ribonucleoprotein DI, Small Nuclear Ribonucleoprotein DI, Polypeptide 16 kDa, Polypeptide (16 kD), SNRPD, HsT2456, Sm-D1, SMD 1, Sm-D Autoantigen, Small Nuclear Ribonucleoprotein D1 Polypeptide 16 kDa Pseudogene, SnRNP Core Protein DI, Small Nuclear Ribonucleoprotein Sm DI; SLC35D3 (Solute Carrier Family 35, Member D3, FRCL1, Fringe Connection-Like Protein 1, bA55K22.3, Frc, Fringe-Like 1, Solute Carrier Family 35 Member D3; NCBI GenBank: NC-000006.11 NC-018917.2 NT-025741.16); SIRT2 (Sirtuin 2, NAD-Dependent Deacetylase Sirtuin-2, SIRL2, Silent Information Regulator 2, Regulatory Protein SIR2 Homolog 2, Sir2-Related Protein Type 2, SIR2-Like Protein 2, Sirtuin Type 2, Sirtuin (Silent Mating Type Information Regulation 2 Homolog) 2 (S. cerevisiae), Sirtuin-2, Sirtuin (Silent Mating Type Information Regulation 2, S. cerevisiae, Homolog) 2, EC 3.5.1., SIR2; GenBank: AAK51133.1); Sema 5b (FLJ10372, KIAA1445, Mm.42015, SEMA5B, SEMAG, Semaphorin 5b Hlog, sema domain, seven thrombospondin repeats (type 1 and type 1-like), Transmembrane Domain.TM. and short cytoplasmic domain, (semaphorin) 5B, GenBank accession no. AB04087; secernin 1 (SCRN1, SES1, KIAA0193, secerin-1; GenBank: EAL24458.1); SAGE (SAGE1, Sarcoma Antigen 1, Cancer/Testis Antigen 14, CT14, Putative Tumor Antigen; NCBI Reference Sequence: NP-061136.2); RU2AS (KAAG1, Kidney Associated Antigen 1, RU2AS, RU2 Antisense Gene Protein, Kidney-Associated Antigen 1; GenBank: AAF23613.1); RNF43-E3 ubiquitin-protein ligase RNF43 precursor [Homo sapiens], RNF 124; URCC; NCBI Reference Sequence: NP-060233.3; RhoC (RGS5 (Regulator Of G-Protein Signaling 5, MSTP032, Regulator Of G-Protein Signalling 5, MSTP092, MST092, MSTP106, MST106, MSTP129, MST129; GenBank: AAB84001.1); RET (ret proto-oncogene; MEN2A; HSCR1; MEN2B; MTC1; PTC; CDHF12; Hs.168114; RET51; RET-ELE; NP-066124.1; NM-020975.4); RBAF600 (UBR4, Ubiquitin Protein Ligase E3 Component N-Recognin 4, Zinc Finger, UBR1 Type 1, ZUBR1, E3 Ubiquitin-Protein Ligase UBR4, RBAF600, 600 KDa Retinoblastoma Protein-Associated Factor, Zinc Finger UBR1-Type Protein 1, EC 6.3.2., N-recognin-4, KIAA0462, p600, EC 6.3.2, KIAA1307; GenBank: AAL83880.1); RAGE-1 (MOK, MOK Protein Kinase, Renal Tumor Antigen, RAGE, MAPK/MAK/MRK Overlapping Kinase, Renal Tumor Antigen 1, Renal Cell Carcinoma Antigen, RAGE-1, EC 2.7.11.22, RAGE1; UniProtKB/Swiss-Prot: Q9UQ07.1); RAB38/NY-MEL-1 (RAB38, NY-MEL-1, RAB38, Member RAS Oncogene Family, Melanoma Antigen NY-MEL-1, Rab-Related GTP-Binding Protein, Ras-Related Protein Rab-38, rrGTPbp; GenBank: AAH15808.1); PTPRK (DJ480114.2.1 (Protein Tyrosine Phosphatase, Receptor Type, K R-PTP-KAPPA, Protein Tyrosine Phosphatase Kappa, Protein Tyrosine Phosphatase Kappa), Protein Tyrosine Phosphatase, Receptor Type, K, Protein-Tyrosine Phosphatase Kappa, Protein-Tyrosine Phosphatase, Receptor Type, Kappa, R-PTP-kappa, Receptor-Type Tyrosine-Protein Phosphatase Kappa, EC 3.1.3.48, PTPK; GenBank: AAI44514.1); PSMA; PSCA hIg(2700050C12Rik, C530008016Rik, RIKEN cDNA 2700050C12, RIKEN cDNA 2700050C12 gene, GenBank accession no. AY358628); PSCA (Prostate stem cell antigen precursor, GenBank accession no. AJ29743; PRDX5 (Peroxiredoxin 5, EC 1.11.1.15, TPx Type VI, B166, Antioxidant Enzyme B166, HEL-S-55, LiverTissue 2D-Page Spot 71 B, PMP20, Peroxisomal Antioxidant Enzyme, PRDX6,Thioredoxin Peroxidase PMP20, PRXV, AOEB 166, Epididymis Secretory Protein Li 55, Alu Co-Repressor 1, Peroxiredoxin-5, Mitochondrial, Peroxiredoxin V, prx-V, Thioredoxin Reductase, Prx-V, ACR1, Alu Corepressor, PLP; GenBank: CAG33484.1); PRAME (Preferentially Expressed Antigen In Melanoma, Preferentially Expressed Antigen Of Melanoma, MAPE, 01P-4, OIPA, CT130, Cancer/Testis Antigen 130, Melanoma Antigen Preferentially Expressed In Tumors, Opa-Interacting Protein 4, Opa-Interacting Protein 01P4; GenBank: CAG30435.1); pml-RARalpha fusion protein; PMEL17 (silver homolog; SILV; D12S53E; PMEL17; SI; SIL); ME20; gp10 BC001414; BT007202; M32295; M77348; NM-006928; PBF (ZNF395, Zinc Finger Protein 395, PRF-1, Huntington disease regulatory, HD Gene Regulatory Region-Binding Protein, Region-Binding Protein 2, Protein 2, Papillomavirus Regulatory Factor 1, HD-Regulating Factor 2, Papillomavirus-Regulatory Factor, PRF1, HDBP-2, Si-1-8-14, HDBP2, Huntington'S Disease Gene Regulatory Region-Binding Protein 2, HDRF-2, Papillomavirus Regulatory Factor PRF-1, PBF; GenBank: AAH01237.1); PAX5 (Paired Box 5, Paired Box Homeotic Gene 5, BSAP, Paired Box Protein Pax-5, B-Cell Lineage Specific Activator, Paired Domain Gene 5, Paired Box Gene 5 (B-Cell Lineage Specific Activator Protein), B-Cell-Specific Transcription Factor, Paired Box Gene 5 (B-Cell Lineage Specific Activator); PAP (REG3A, Regenerating Islet-Derived 3 Alpha, INGAP, PAP-H, Hepatointestinal Pancreatic Protein, PBBCGF, Human Proislet Peptide, REG-Ill, Pancreatitis-Associated Protein 1, Regi, Reg Ill-Alpha, hepatocarcinoma-intestine-pancreas, Regenerating Islet-Derived Protein Ill-Alpha, Pancreatic Beta Cell Growth Factor, HIP, PAP Homologous Protein, HIP/PAP, Proliferation-Inducing Protein 34, PAP1, Proliferation-Inducing Protein 42, REG-3-alpha, Regenerating Islet-Derived Protein 3-Alpha, Pancreatitis-Associated Protein; GenBank: AAH36776.1); p53 (TP53, Tumor Protein P53, TPR53, P53, Cellular Tumor Antigen P53, Antigen NY-CO-13, Mutant Tumor Protein 53, Phosphoprotein P53, P53 Tumor Suppressor, BCC7, Transformation-Related Protein 53, LFS1, tumor Protein 53, Li-Fraumeni Syndrome, Tumor Suppressor P53; P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), al: 7.63, MW: 47206 TM: 1 [P] Gene Chromosome: 17p13.3, GenBank accession No. NP-002552.; OGT (O-Linked N-Acetylglucosamine (GlcNAc) Transferase, O-GlcNAc Transferase P110 Subunit, O-Linked N-Acetylglucosamine (GlcNAc) Transferase (UDP-N-Acetylglucosamine: Polypeptide-N-Acetylglucosaminyl Transferase, UDP-N-Acetylglucosamine-Peptide N-Acetylglucosaminyltransferase 110 KDa Subunit, UDP-N-Acetylglucosamine: Polypeptide-N-Acetylglucosaminyl Transferase, Uridinediphospho-N-Acetylglucosamine:Polypeptide Beta-N-Acetylglucosaminyl Transferase, O-GlcNAc Transferase Subunit P110, EC 2.4.1.255, O-Linked N-Acetylglucosamine Transferase 110 KDa Subunit, EC 2.4.1, HRNT1, EC 2.4.1.186, O-GLCNAC; GenBank: AAH38180.1); OA1 (Osteoarthritis QTL 1, OASD; GenBank: CAA88742.1); NY-ESO-1/LAGE-2 (Cancer/Testis Antigen 1 B, CTAG1 B, NY-ESO-1, LAGE-2, ESO1, CTAG1, CTAG, LAGE2B, Cancer/Testis Antigen 1, Autoimmunogenic Cancer/Testis Antigen NY-ESO-1, Ancer Antigen 3, Cancer/Testis Antigen 6.1, New York Esophageal Squamous Cell Carcinoma 1, L Antigen Family Member 2, LAGE2, CT6.1, LAGE2A; GenBank: AA130365.1); NY-BR-1 (ANKRD30A, Ankyrin Repeat Domain 30A, Breast Cancer Antigen NY-BR-1, Serologically Defined Breast Cancer Antigen NY-BR-1, Ankyrin Repeat Domain-Containing Protein 30A; NCBI Reference Sequence: NP-443723.2); N-ras (NRAS, Neuroblastoma RAS Viral (V-Ras) Oncogene Homolog, NRAS 1, Transforming Protein N-Ras, GTPase NRas, ALPS4, N-Ras Protein Part 4, NS6, Oncogene Homolog, HRAS 1; GenBank: AAH05219.1); NFYC (Nuclear Transcription Factor Y, Gamma, HAPS, HSM, Nuclear Transcription Factor Y Subunit C, Transactivator HSM-1/2, CCAAT Binding Factor Subunit C, NF-YC, CCAAT Transcription Binding Factor Subunit Gamma, CAAT Box DNA-Binding Protein Subunit C, Histone H1 Transcription Factor Large Subunit 2A, CBFC, Nuclear Transcription Factor Y Subunit Gamma, CBF-C, Transactivator HSM-1, H1TF2A, Transcription Factor NF-Y, C Subunit; neo-PAP (PAPOLG, Poly(A) Polymerase Gamma, Neo-Poly(A) Polymerase, Nuclear Poly(A) Polymerase Gamma, Polynucleotide Adenylyltransferase Gamma, SRP RNA 3 Adenylating Enzyme/Pap2, PAP-gamma, Neo-PAP, SRP RNA 3′-Adenylating Enzyme, PAP2, EC 2.7.7.19, PAPG; NCBI Reference Sequence: NP-075045.2); NCA (CEACAM6, GenBank accession no. M1872); Napi3b (NAPI-3B, NPTIIb, SLC34A2, solute carrier family 34 (sodium phosphate), member 2, type II sodium-dependent phosphate transporter 3b, GenBank accession no. NM-00642); Myosin class I; MUM-3; MUM-2 (TRAPPC1, Trafficking Protein Particle Complex 1, BETS, BETS Homolog, MUM2, Melanoma Ubiquitous Mutated 2, Multiple Myeloma Protein 2, Trafficking Protein Particle Complex Subunit 1; MUM-if; Mucin (MUC1, Mucin 1, Cell Surface Associated, PEMT, PUM, CA 15-3, MCKD1, ADMCKD, Medullary Cystic Kidney Disease 1 (Autosomal Dominant), ADMCKD1, Mucin 1, Transmembrane, CD227, Breast Carcinoma-Associated Antigen DF3, MAM6, Cancer Antigen 15-3, MCD, Carcinoma-Associated Mucin, MCKD, Krebs Von Den Lungen-6, MUC-1/SEC, Peanut-Reactive Urinary Mucin, MUC1/ZD, Tumor-Associated Epithelial Membrane Antigen, DF3 Antigen, Tumor-Associated Mucin, episialin, EMA, H23 Antigen, H23AG, Mucin-1, KL-6, Tumor Associated Epithelial Mucin, MUC-1, Episialin, PEM, CD227 Antigen; UniProtKB/Swiss-Prot: P15941.3); MUCSAC (Mucin SAC, Oligomeric Mucus/Gel-Forming, Tracheobronchial Mucin′ MUC5, TBM, Mucin 5, Subtypes A And C, Tracheobronchial/Gastric, leB, Gastric Mucin, Mucin SAC, Oligomeric Mucus/Gel-Forming Pseudogene, Lewis B Blood Group Antigen, LeB, Major Airway Glycoprotein, MUC-SAC, Mucin-5 Subtype AC, Tracheobronchial; MUC1 (Mucin 1, Cell Surface Associated, PEMT, PUM, CA 15-3, MCKD1, ADMCKD, Medullary Cystic Kidney Disease 1 (Autosomal Dominant), ADMCKD1, Mucin 1, Transmembrane, CD227, Breast Carcinoma-Associated Antigen DF3, MAM6, Cancer Antigen 15-3, MCD, Carcinoma-Associated Mucin, MCKD, Krebs Von Den Lungen-6, MUC-1/SEC, Peanut-Reactive Urinary Mucin, MUC-1/X, Polymorphic Epithelial Mucin, MUC 1/ZD, Tumor-Associated Epithelial Membrane Antigen, DF3 Antigen, Tumor-Associated Mucin, episialin, EMA, h23 Antigen, H23AG, mucin-1, KL-6, Tumor Associated Epithelial Mucin, MUC-1, Episialin, PEM, CD227 Antigen; MSG783 (RNF 124, hypothetical protein FU20315, GenBank accession no. NM-01776; MRP4-multidrug resistance-associated protein 4 isoform 3, MOAT-B; MOATB [Homo sapiens]; NCBI Reference Sequence: NP-001288758.1; MPF (MPF, MSLN, SMR, megakaryocyte potentiating factor, mesothelin, GenBank accession no. NM-00582; MMP-7 (MMP7, matrilysin, MPSL1, matrin, Matrix Metalloproteinase 7 (Matrilysin, Uterine), Uterine Matrilysin, Matrix Metalloproteinase-7, EC 3.4.24.23, Pump-1 Protease, Matrin, Uterine Metalloproteinase, PUMP1, MMP-7, EC 3.4.24, PUMP-1; GenBank: AAC37543.1); MMP-2 (MMP2, Matrix Metallopeptidase 2 (Gelatinase A, 72 kDa Gelatinase, 72 kDa Type IV Collagenase), MONA, CLG4A, Matrix Metalloproteinase 2 (Gelatinase A, 72 kD Gelatinase, 72 kD Type IV Collagenase), CLG4, 72 kDa Gelatinase, 72 kDa Type IV Collagenase), Matrix Metalloproteinase-2, MMP-II, 72 KDa Gelatinase, Collagenase Type IV-A, MMP-2, Matrix Metalloproteinase-II, TBE-1, Neutrophil Gelatinase, EC 3.4.24.24, EC 3.4.24; GenBank: AAH02576.1); Meloe; 17-IA, 4-1BB, 4Dc, 6-keto-PGFIa, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, API, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCLII, CCL12, CCL13, CCL 14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CDIIa, CDIIb, CDIIc, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxinl, EpCAM, Ephrin B2/EphB4, EPO, ERCC, E-selectin, ET-1, Factor Ila, Factor VII, Factor Vilic, Factor IX, fibroblast activation protein (FAP), Fas, FcRI, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alphal, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/betal, integrin, alpha4/beta7, integrin alpha5 (alphaV), integrin alpha5/betal, integrin alpha5/beta3, integrin alpha6, integrin betal, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein LI, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mucl), MUC 18, Muellerian-inhibitin substance, Mug, MuSK, NAIP, NAP, NCAD, N-Cadherin, NCA 90, NCAM, NCAM, Neprilysin, Neurotrophin-3,-4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S 100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta Rllb, TGF-beta Rill, TGF-betal, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSFIOB (TRAIL R2 DR5, KILLER, TRICK-2A, TRICK-B), TNFRSF10C (TRAIL R3 DcRI, LIT, TRID), TNFRSF10D (TRAIL R4 DcR2, TRUNDD), TNFRSF11A (RANK ODF R, TRANCE R), TNFRSFIIB (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF 18 (GITR AITR), TNFRSF 19 (TROY TAJ, TRADE), TNFRSF 19L (RELT), TNFRSFIA (TNF RI CD120a, p55-60), TNFRSFIB (TNF RII CD120b, p⁷⁵-8⁰), TNFRSF26 (TNFRH3), TNFRSF3 (LTbR TNF Rill, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF 12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF 18 (GITR Ligand AITR Ligand, TL6), TNFSFIA (TNF-α Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VIM, Viral antigens, VLA, VLA-1, VLA-4, VNR integrin, von Willebrand's factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), receptors for hormones, and growth factors. In certain embodiments, CAR may have specificity for BCMA, CTLA4 (cytotoxic T lymphocyte antigen-4), PD 1 (programmed cell death protein 1), PD-L1 (programmed cell death ligand 1), LAG-3 (lymphocyte activation gene-3), TIM-3, CD20, CD2, CD19, Her2, EGFR, EpCAM, FcγRIIIa (CD16), FcγRIIa (CD32a), FcγRIIb (CD32b), FcγRI (CD64), Toll-like receptors (TLRs), TLR4, TLR9, cytokines, IL-2, IL-5, IL-13, IL-6, IL-17, IL-12, IL-23, TNFa, TGFb, cytokine receptors, IL-2R, chemokines, chemokine receptors, growth factors, VEGF, and HGF. CAR-T cells may be used in combination with NEO-201 to treat any cancer, infectious, inflammatory or autoimmune condition wherein CAR-T cells find application as above-described.

NEO-201 Combined with CAR-NK Cell Therapies

NEO-201, because of its ability to ablate gMDSCs, should also improve the efficacy of CAR-NK cell therapies. Particularly, the great success of CAR-T therapy in clinical trials has led to the development of CAR-NK cells. Extracellular, transmembrane and intracellular signaling domains are present in CAR-NK cells as they are in CAR-Tcells. CAR-NK cells often have CD3 as their initial signaling domain and CD28 or CD137 (4-1BB) as a costimulatory domain to form an intracellular signaling motif. NK cells increase their cytotoxic capability and cytokine production through two more costimulatory molecules, namely, NKG2D and CD244 (2B4). Owing to more enhanced tumor-specific targeting and cytotoxicity than those of CAR-T cells, CAR-modified NK cells have often been used to target cancer cells.

CAR-NK cell therapies possess advantageous features such as low safety concerns, low costs, and higher tumor potential than CAT-T cells. Allogeneic haploidentical NK cells are safe for adoptive cell therapy (ACT) because they usually do not mediate and may diminish GVHD. Also, CAR-NK cells have considerably fewer safety concerns than CAR-T cells such as on-target/off-tumor effects, CRS and tumor lysis syndrome. Moreover, NK cells only secrete a small number of IFN-γ and GM-CSF and do not produce IL-1 and IL-6 that initiate CRS. Second, tumor cells may not be detected by CAR-T cells owing to tumor escape because of a loss of either MHC class I expression or tumor-specific antigens. CAR-NK cells lack a self-antigen and can detect MHC class I-negative tumor cells because they retain innate cytotoxic potential against germline-encoded tumor/stress ligands. In addition, both HLA-A and HLA-B bind to KIR3D receptors, whereas HLA-C only binds to KIR2D receptors. CD94-NKG2A, which detects HLA-E, LILRB1 and all MHC class I molecules, is another inhibitory receptor that identifies MHC class I molecules expressed by NK cells. Normal MHC class I-sufficient cells are ignored by NK cells because their inhibitory receptors can detect MHC class I molecules; however, they are not inhibited after interacting with abnormal MHC class I low cells. Third, it is believed that low levels of MHC class I expression in cancer stem cells (CSCs) and the presence of NKp30, NKp44 and NKG2D (activating receptors) cause cytokine-activated NK cell-mediated death of CSCs. Fourth, CAR-NK cells can regulate their activating receptors, including NKp30, NKp44, NKp46, NKG2D, KIR-2DS, KIR-3DS, 2B4, CD226, CD94/NKG2C and DNAM-1; therefore, the chances of relapse owing to the loss of CAR-targeting antigens is reduced. Moreover, T lymphocytes only kill their targets through a CAR-specific mechanism, whereas NK cells exhibit spontaneous cytotoxic activity and can kill target cells regardless of the presence of tumor-specific antigens. Tumor cells downregulate antigens to escape immune detection; however, NK cells are still effective against them. Furthermore, cytokines such as IFN-γ, IL-3 and GM-CSF produced by primary human NK cells are different from proinflammatory cytokines released by T cells, which induce CRS. Individual NK cells can survive after interacting with and destroying several target cells, potentially decreasing the number of cells that are adoptively transferred. Fifth, the availability of an off-the-shelf CAR-NK therapy enhances the pace of administration remarkably and first dosing to 1 day by minimizing the lag time from the decision to treat. Sixth, CAR-NK therapy should decrease huge indirect costs because CAR-NK infusions can be administered with outpatient follow-up monitoring and do not require lengthy post-treatment hospitalization because they are safer and have no potential toxicity. In addition, NK cells can be harvested from multiple sources including iPSCs, PB, UCB, human embryonic stem cells and NK cell lines. Like CAR-T cells, CAR-NK cell therapy is being used to treat hematological and solid tumors. CD19 (NCT02742727), CD7 (NCT02742727) and CD33 (NCT02944162) are targets for CAR-NK cell therapy used in reported clinical studies on lymphoma and leukemia. Also, HER2-targeted GBM (NCT03383978) and costimulating conversion receptors are being used to treat non-small-cell lung carcinoma (NSCLC) (NCT03656705). CAR-NK cell therapy against multiple refractory solid tumors targeting mucin 1 (MUC1), including pancreatic tumors, HCC, NSCLC and triple-negative invasive breast tumors, is also under investigation (NCT02839954).

CAR-NK cells may be used in combination with NEO-201 to treat any cancer, infectious, inflammatory or autoimmune condition wherein CAR-T cells are used as above-described. The CAR expressed by such NK cells may be specific to any of the antigens targeted by CAR-T cells. Also, the CAR may comprise any of the signaling, hinge, and other domains that are typically used in CARs which are expressed in CAR-T cells. Such domains and sequences used in CARs are generally known in the art and are above-described.

### Monitoring/Detection of MDSC in Patients

In some embodiments gMDSCs in the patient will be detected and monitored prior, during and after treatment has been completed or after the patient has gone into remission. Such methods may be useful in determining whether the patient will potentially benefit from NEO-201 treatment.

Methods for detection and monitoring of gMDSCs in patient samples are known in the art and are disclosed in US published application 20210318310 by Gabrilovich; Dmitry I., published on Oct. 14, 2021; US published application 20170261507 by BANIYASH; Michal published on Sep. 14, 2017 which applications are incorporated by reference in their entirety.

Methods for identifying and separating gMDSCs from a sample can include contacting the biological sample with ligands, e.g., antibodies that recognize specific biomarkers expressed on gMDSCs. Such biomarkers include LOX-1, CD11b, CD15, and CD66b.

These methods may provide an accurate enumeration or concentration of a gMDSC cell population from a suitable biological sample of a subject.

In some embodiments, these methods of determining an accurate cell count/concentration of gMDSCs in a subject having a cancer or being treated for a cancer with NEO-201 alone or in combination with another therapeutic agent can be used to monitor the progression of the cancer (with or without treatment).

In some embodiments, these methods of determining gMDSC numbers or concentration in a subject with cancer may be used to determine whether NEO-201 may be beneficial in treating the cancer, alone or in combination with another therapeutic agent.

In some embodiments, these methods of determining gMDSC numbers or concentration in a tumor may be used to develop a dosing regimen of NEO-201 alone or in combination with another therapeutic agent.

In some embodiments, the disclosure provides a method of detecting gMDSCs, wherein the level of gMDSCs in a patient sample, such as a blood or biopsy sample, is used to determine cancer prognosis prior, during or after NEO-201 treatment. For example, the patient may be assigned to be administered or may be administered NEO-201 in an amount effective to kill gMDSCs if gMDSCs reactive to NEO-201 cells are detected in said patient sample. Said method may comprise contacting said gMDSCs with a NEO-201 antibody.

Said detecting may comprise cell sorting, optionally fluorescence activated cell sorting, thereby producing a sample enriched for and/or depleted of cells positive for NEO-201 antigen expression, e.g., gMDSCs.

In another aspect, the disclosure provides a method of detecting gMDSCs, comprising contacting cells with a NEO-201 antibody and detecting cells that express NEO-201 target antigen. Said NEO-201 antibody may be directly or indirectly labeled.

In another aspect, the disclosure provides a method of staining gMDSCs, comprising contacting cells with a NEO-201 antibody. Said NEO-201 antibody may be directly or indirectly labeled.

In another aspect, the disclosure provides a method of isolating or enriching MDSCs, comprising isolating cells that express the NEO-201 target antigen. Said method may comprise contacting a sample, e.g., a tumor biopsy sample containing gMDSCs with a NEO-201 antibody, optionally wherein said NEO-201 antibody is directly or indirectly labeled. Said sample may also comprise blood or bone marrow. Said method may comprise separating NEO-201 positive gMDSCs from NEO-201 negative cells. Said method may further comprise conducting further diagnostic assays on said cell sample to detect expression of other MDSC biomarkers.

Said gMDSCs also may be isolated by cell sorting, optionally fluorescence activated cell sorting, based on NEO-201 target antigen expression and the expression of other MDSC biomarkers.

Said gMDSCs may be isolated by contacting sample with a support comprising a NEO-201 antibody and/or using other antibodies or ligands which recognize other MDSC biomarkers, whereby said MDSCs are retained on said support.

In another aspect, the disclosure provides a method of detecting gMDSCs, comprising detecting the expression of the NEO-201 target antigen by said MDSCs, optionally wherein the level of gMDSCs in a patient sample, such as a blood or biopsy sample, is used to determine whether a patient has or likely to develop MDSC-mediated immunosuppression. Optionally said method may further comprise assigning or administering NEO-201 treatment to a patient based on the detection of said gMDSCs. For example, the patient may be assigned to be administered or may be administered NEO-201 in an amount effective to kill gMDSCs if gMDSCs reactive to NEO-201 and/or other biomarkers are detected in said patient sample. Said method may comprise contacting said gMDSCs with a NEO-201 antibody.

Said detecting may comprise cell sorting, optionally fluorescence activated cell sorting, thereby producing a sample enriched for and/or depleted of cells positive for NEO-201 target antigen expression, e.g., gMDSCs.

In another aspect, the disclosure provides a method of detecting gMDSCs, comprising contacting cells with a NEO-201 antibody and detecting cells that express NEO-201 target antigen. Said NEO-201 antibody may be directly or indirectly labeled.

In another aspect, the disclosure provides a method of staining gMDSCs, comprising contacting cells with a NEO-201 antibody. Said NEO-201 antibody may be directly or indirectly labeled.

In another aspect, the disclosure provides a method of isolating gMDSCs, comprising isolating cells that express the NEO-201 target antigen and optionally other MDSC biomarkers. Said method may comprise contacting a sample containing a cell sample, e.g., a tumor biopsy sample, with a NEO-201 antibody, optionally wherein said NEO-201 antibody is directly or indirectly labeled. Said sample may alternatively comprise a blood or bone marrow sample. Said method may comprise separating NEO-201 positive gMDSCs from NEO-201 negative cells. Said method may further comprise conducting further diagnostic assays on said putative gMDSCs e.g., using ligands that bind to other MDSC biomarkers.

Said gMDSCs may be isolated by cell sorting, optionally fluorescence activated cell sorting, based on NEO-201 expression.

Said gMDSCs may be isolated by contacting sample with a support comprising a NEO-201 antibody, whereby said gMDSCs are retained on said support.

### Cancer Vaccines

The subject treatment methods may further comprise administering a cancer vaccine to said patient. Exemplary cancer vaccines that may be administered are disclosed in, e.g., Fisher et al., Immun Inflamm Dis. 2017 March; 5(1): 16-28; Klages et al., Cancer Res Oct. 15, 2010 (70) (20) 7788-7799; Reginato et al., Br J Cancer. 2013 Oct. 15; 109(8): 2167-2174; Litzinger M T et al., Blood 2007, 110:3192, each of which is hereby incorporated by reference in its entirety.

In Vitro Ablation of gMDSCs Using NEO-201

In another aspect, the disclosure provides a method of killing gMDSC cells in vitro, comprising contacting said gMDSC cells with a NEO-201 antibody. Said method may further comprise contacting said gMDSC cells with complement. Said gMDSC cells may be killed by CDC. Said method may further comprise contacting said gMDSC with effector cells, such as natural killer cells. Said gMDSCs may be killed by ADCC.

In another aspect, the disclosure provides a method of killing MDSC ex vivo, comprising contacting a sample comprising gMDSCs with an effective amount of a NEO-201 antibody. Said sample may be obtained from a patient. Also, in some instances the NEO-201 antibody may be coupled to a cytotoxic moiety.

### NEO-201 Antibody Sequences

In any of the foregoing or following methods, said NEO-201 antibody may comprise at least one, two, three, four, five, or preferably all six of the CDR sequences contained in SEQ ID NO: 28 and SEQ ID NO: 29.

In any of the foregoing or following methods, said NEO-201 antibody may comprise a variable heavy chain sequence having at least 90% identity to SEQ ID NO: 38.

In any of the foregoing or following methods, said NEO-201 antibody may comprise a variable light chain sequence having at least 90% identity to SEQ ID NO: 39.

In any of the foregoing or following methods, said NEO-201 antibody may comprise a variable heavy chain sequence having at least 90% identity to SEQ ID NO: 38 and a variable light chain sequence having at least 90% identity to SEQ ID NO: 39.

In any of the foregoing or following methods, said NEO-201 antibody may comprise a heavy chain sequence having at least 90% identity to amino acids 20-470 of SEQ ID NO: 28 and a light chain sequence having at least 90% identity to amino acids 20-233 of SEQ ID NO: 29.

In any of the foregoing or following methods, said NEO-201 antibody may comprise all six of the CDR sequences contained in SEQ ID NO: 28 and SEQ ID NO: 29.

In any of the foregoing or following methods, said NEO-201 antibody may comprise a human IgG1 constant domain. Alternatively, said NEO-201 antibody may comprise a human IgG2, human IgG3, or human IgG4 constant domain, or a hybrid or chimeric domain comprising two or more of human IgG1, IgG2, IgG3, or IgG4.

In any of the foregoing or following methods, the antibody comprises the NEO-201 antibody or a variant thereof, e.g., one comprising the same CDRs and/or variable regions as NEO-201.

In any of the foregoing or following methods, said NEO-201 antibody may be conjugated to another moiety.

In any of the foregoing or following methods, said NEO-201 antibody may be conjugated to another cytotoxic moiety, label, radioactive moiety, or affinity tag.

In any of the foregoing or following methods, said NEO-201 antibody may compete with the antibody contained in SEQ ID NO: 28 and SEQ ID NO: 29 for binding to the NEO-201 antigen.

### Definitions

Unless defined otherwise, all technical and scientific terms used herein have the same meaning as those commonly understood by one of ordinary skill in the art to which this invention belongs. Although methods and materials similar or equivalent to those described herein may be used in the invention or testing of the present invention, suitable methods and materials are described herein. The materials, methods and examples are illustrative only, and are not intended to be limiting.

As used in the description herein and throughout the claims that follow, the meaning of “a,” “an,” and “the” includes plural reference unless the context clearly dictates otherwise.

“Amino acid,” as used herein refers broadly to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The terms “NK-depleted” or “natural killer-depleted” as used herein refer to a patient having low natural killer (NK) cell levels relative to the normal range. NK cells are a cytotoxic innate immune lymphocyte. Typically, NK cells comprise 5-20% of the peripheral blood mononuclear cells (PBMCs) in a healthy individual. A patient having NK cells comprising less than 5% of the PMBCs is referred to as NK-depleted. Additionally, a patient is referred to as severely NK-cell depleted if NK cells comprising less than 3% of the PMBCs. Additionally, in normal individuals, up to 90% of PBMC NK cells are CD56^{dim}CD16⁺ NK cells, and these are considered the most cytotoxic subset. If less than 70% of PBMC NK cells are CD56^{dim}CD16⁺ NK cells, then the patient is referred to as NK-depleted. Additionally, if less than 50% of PBMC NK cells are CD56^{dim}CD16⁺ NK cells, then the patient is referred to as severely NK-depleted. A given patient may be referred to as NK-depleted or severely NK-depleted based on meeting either or both of these individual criteria. Generally speaking, a patient's status as NK-depleted or severely NK-depleted is determined by testing a sample taken from the patient, e.g., a blood sample, e.g., a sample obtained and tested within one or two weeks prior. A patient's status as NK-depleted or severely NK-depleted may also be inferred from a disease diagnosis and/or a course of treatment that is associated with such depletion of NK cells.

“Antibody,” as used herein, refers broadly to any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, IgG, IgM, IgA, IgE, IgD, from all sources, e.g., human, rodent, rabbit, cow, sheep, pig, dog, chicken, are considered to be “antibodies”. Antibodies include but are not limited to chimeric antibodies, human antibodies and other non-human mammalian antibodies, humanized antibodies, single chain antibodies (scFvs), camelbodies, nanobodies, IgNAR (single-chain antibodies derived from sharks), small-modular immunopharmaceuticals (SMIPs), and antibody fragments (e.g., Fabs, Fab′, F(ab′)₂). Numerous antibody coding sequences have been described; and others may be raised by methods well-known in the art. See Streltsov, et al. (2005) Protein Sci. 14(11): 2901-9; Greenberg, et al. (1995) Nature 374(6518): 168-173; Nuttall, et al. (2001) Mol Immunol. 38(4): 313-26; Hamers-Casterman, et al. (1993) Nature 363(6428): 446-8; Gill, et al. (2006) Curr Opin Biotechnol. 17(6): 653-8.

“NEO-201 antibody” refers to an antibody containing the heavy and light chains of SEQ ID NOs: 28 and 29 or the variable regions optionally together with the constant regions contained therein, as well as fragments and variants thereof. Such variants include sequences containing one, two, three, four, five or preferably all six of the CDR sequences contained in SEQ ID NO: 28 and SEQ ID NO: 29, i.e., the heavy chain CDR1 of SEQ ID NO: 32, the heavy chain CDR2 of SEQ ID NO: 33, the heavy chain CDR3 of SEQ ID NO: 34, the light chain CDR1 of SEQ ID NO: 35, the light chain CDR2 of SEQ ID NO: 36, and the light chain CDR3 of SEQ ID NO: 37. Such variants also include antibodies that compete with NEO-201 for binding to the NEO-201 antigen. Said antibody may be humanized. Said antibody may be expressed containing one or more leader sequences, which may be removed during expression and/or processing and secretion of the antibody. Said antibody may be presented in a monovalent, bivalent, or higher multivalent format, including without limitation a bispecific or multispecific antibody containing said NEO-201 antibody sequence and a binding fragment of a different antibody. Typically said antibody specifically binds to carcinoma cells and competes for binding to carcinoma cells with an antibody comprising the variable heavy chain of SEQ ID NO: 38 and variable light chain of SEQ ID NO: 39, or comprising the heavy chain of SEQ ID NO: 28 and light chain of SEQ ID NO: 29. One or more of those CDR sequences contained in SEQ ID NO: 28 and/or SEQ ID NO: 29 may be substituted with a variant sequence, such as the light chain CDR1 of SEQ ID NO: 1 or 4; light chain CDR2 of SEQ ID NO: 2 or 5; light chain CDR3 of SEQ ID NO: 3 or 6; heavy chain CDR1 of SEQ ID NO: 7; heavy chain CDR2 of SEQ ID NO: 8,10, 30, or 31; heavy chain CDR3 of SEQ ID NO: 9 or 11; or SEQ ID NOs: 30-31. The light chain may comprise the CDRs contained in the light chain sequence of SEQ ID NO: 14, 16, 17, 18, 19, 20, 21, or 29. The heavy chain may comprise the CDRs contained in the heavy chain sequence of SEQ ID NO: 15, 22, 23, 24, 25, 26, 27, or 29. Said antibody may comprise a variable heavy chain sequence having at least 75%, 80%, 85%, 90%, 95%, 96%, 97%, 98%, or 99% identity to SEQ ID NO: 38, and/or a variable light chain sequence having at least 75%, 80%, 85%, 90%, 95%, 96%, 97%, 98%, or 99% identity to SEQ ID NO: 39, optionally wherein said heavy and/or light chain sequence contains one, two, three, four, five or preferably all six of the CDR sequences contained in SEQ ID NO: 28 and SEQ ID NO: 29, i.e., the heavy chain CDR1 of SEQ ID NO: 32, the heavy chain CDR2 of SEQ ID NO: 33, the heavy chain CDR3 of SEQ ID NO: 34, the light chain CDR1 of SEQ ID NO: 35, the light chain CDR2 of SEQ ID NO: 36, and the light chain CDR3 of SEQ ID NO: 37. Said antibody may be conjugated to another moiety, such as a cytotoxic moiety, radioactive moiety, label, or purification tag.

“Antigen,” as used herein, refers broadly to a molecule or a portion of a molecule capable of being bound by an antibody which is additionally capable of inducing an animal to produce an antibody capable of binding to an epitope of that antigen. An antigen may have one epitope, or have more than one epitope. The specific reaction referred to herein indicates that the antigen will react, in a highly selective manner, with its corresponding antibody and not with the multitude of other antibodies which may be evoked by other antigens. Antigens may be tumor specific (e.g., expressed by neoplastic cells of pancreatic and colon carcinoma.)

“Cancer,” as used herein, refers broadly to any neoplastic disease (whether invasive or metastatic) characterized by abnormal and uncontrolled cell division causing malignant growth or tumor.

“Cancer vaccine,” as used herein, refers to an immunogenic composition that elicits or is intended to elicit an immune response against a cancer cell.

“Chimeric antibody,” as used herein, refers broadly to an antibody molecule in which the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug; or the variable region, or a portion thereof, is altered, replaced or exchanged with a variable region having a different or altered antigen specificity.

“Conservatively modified variants,” as used herein, applies to both amino acid and nucleic acid sequences, and with respect to particular nucleic acid sequences, refers broadly to conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) may be modified to yield a functionally identical molecule.

“Complementarity determining region,” “hypervariable region,” or “CDR,” as used herein, refers broadly to one or more of the hyper-variable or complementarily determining regions (CDRs) found in the variable regions of light or heavy chains of an antibody. See Kabat, et al. (1987) “Sequences of Proteins of Immunological Interest” National Institutes of Health, Bethesda, MD. These expressions include the hypervariable regions as defined by Kabat, et al. (1983) “Sequences of Proteins of Immunological Interest” U.S. Dept. of Health and Human Services or the hypervariable loops in 3-dimensional structures of antibodies. Chothia and Lesk (1987) J Mol. Biol. 196: 901-917. The CDRs in each chain are held in close proximity by framework regions and, with the CDRs from the other chain, contribute to the formation of the antigen binding site. Within the CDRs there are select amino acids that have been described as the selectivity determining regions (SDRs) which represent the critical contact residues used by the CDR in the antibody-antigen interaction. Kashmiri (2005) Methods 36: 25-34.

“Control amount,” as used herein, refers broadly to a marker can be any amount or a range of amounts to be compared against a test amount of a marker. For example, a control amount of a marker may be the amount of a marker in a patient with a particular disease or condition or a person without such a disease or condition. A control amount can be either in absolute amount (e.g., microgram/ml) or a relative amount (e.g., relative intensity of signals).

“Differentially present,” as used herein, refers broadly to differences in the quantity or quality of a marker present in a sample taken from patients having a disease or condition as compared to a comparable sample taken from patients who do not have one of the diseases or conditions. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker may be considered to be differentially present. Optionally, a relatively low amount of up-regulation may serve as the marker.

“Diagnostic,” as used herein, refers broadly to identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives”. Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Diagnosing,” as used herein refers broadly to classifying a disease or a symptom, determining a severity of the disease, monitoring disease progression, forecasting an outcome of a disease and/or prospects of recovery. The term “detecting” may also optionally encompass any of the foregoing. Diagnosis of a disease according to the present invention may, in some embodiments, be affected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject.

“Effective amount,” as used herein, refers broadly to the amount of a compound, antibody, antigen, or cells that achieves a desired result. An “effective amount” when administered to a patient for treating a disease, is sufficient to effect such treatment for the disease. The effective amount may be an amount effective for prophylaxis, and/or an amount effective for prevention. The effective amount may be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” may vary depending on the disease and its severity and the age, weight, medical history, susceptibility, and preexisting conditions, of the patient to be treated. The term “effective amount” is synonymous with “therapeutically effective amount” for purposes of this disclosure.

“Expression vector,” as used herein, refers broadly to any recombinant expression system for the purpose of expressing a nucleic acid sequence of the present disclosure in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression cassettes which contain only the minimum elements needed for transcription of the recombinant nucleic acid.

“Framework region” or “FR,” as used herein, refers broadly to one or more of the framework regions within the variable regions of the light and heavy chains of an antibody. See Kabat, et al. (1987) “Sequences of Proteins of Immunological Interest,” National Institutes of Health, Bethesda, MD. These expressions include those amino acid sequence regions interposed between the CDRs within the variable regions of the light and heavy chains of an antibody.

“Hematological malignancy” refers to forms of cancer that begin in blood-forming tissue, such as the bone marrow, or in the cells of the immune system. Examples of hematological malignancies include leukemia, lymphoma, multiple myeloma, and myelodysplastic syndromes (MDS). More specific examples of hematological malignancies include but are not limited to marginal zone lymphoma (MZL) (including splenic marginal zone lymphoma (SMZL)), Burkitt lymphoma (BL), multiple myeloma (MM) (including plasma cell leukemia (PCL) and myeloma extramedullary disease (EMD)), myelodysplastic syndromes (MDS), acute myeloid leukemia (AML) (including B-cell AML), acute lymphocytic leukemia (ALL), T-cell lymphoma (TCL) (including anaplastic large cell lymphoma (ALCL) and Sezary Syndrome), and Hodgkin's lymphoma (HL).

“Heterologous,” as used herein, refers broadly to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

“High affinity,” as used herein, refers broadly to an antibody having a KD of at least 10⁻⁸ M, more preferably at least 10⁻⁹ M and even more preferably at least 10⁻¹⁰ M for a target antigen. However, “high affinity” binding can vary for other antibody isotypes. For example, “high affinity” binding for an IgM isotype refers to an antibody having a KD of at least 10⁻⁷ M, more preferably at least 10⁻⁸ M.

“Homology,” as used herein, refers broadly to a degree of similarity between a nucleic acid sequence and a reference nucleic acid sequence or between a polypeptide sequence and a reference polypeptide sequence. Homology may be partial or complete. Complete homology indicates that the nucleic acid or amino acid sequences are identical. A partially homologous nucleic acid or amino acid sequence is one that is not identical to the reference nucleic acid or amino acid sequence. The degree of homology can be determined by sequence comparison. The term “sequence identity” may be used interchangeably with “homology.”

“Host cell,” as used herein, refers broadly to a cell that contains an expression vector and supports the replication or expression of the expression vector. Host cells may be prokaryotic cells such as E. coli, or eukaryotic cells such as yeast, insect (e.g., SF9), amphibian, or mammalian cells such as CHO, HeLa, HEK-293, e.g., cultured cells, explants, and cells in vivo.

“Hybridization,” as used herein, refers broadly to the physical interaction of complementary (including partially complementary) polynucleotide strands by the formation of hydrogen bonds between complementary nucleotides when the strands are arranged antiparallel to each other.

“K-assoc” or “Ka”, as used herein, refers broadly to the association rate of a particular antibody-antigen interaction, whereas the term “Kdiss” or “Kd,” as used herein, refers to the dissociation rate of a particular antibody-antigen interaction. The term “KD”, as used herein, is intended to refer to the dissociation constant, which is obtained from the ratio of Kd to Ka (i.e., Kd/Ka) and is expressed as a molar concentration (M). KD values for antibodies can be determined using methods well established in the art.

“Immunoassay,” as used herein, refers broadly to an assay that uses an antibody to specifically bind an antigen. The immunoassay may be characterized by the use of specific binding properties of a particular antibody to isolate, target, and/or quantify the antigen.

“Isolated,” as used herein, refers broadly to material removed from its original environment in which it naturally occurs, and thus is altered by the hand of man from its natural environment. Isolated material may be, for example, exogenous nucleic acid included in a vector system, exogenous nucleic acid contained within a host cell, or any material which has been removed from its original environment and thus altered by the hand of man (e.g., “isolated antibody”).

“Label” or a “detectable moiety” as used herein, refers broadly to a composition detectable by spectroscopic, photochemical, biochemical, immunochemical, chemical, or other physical means.

“Low stringency,” “medium stringency,” “high stringency,” or “very high stringency conditions,” as used herein, refers broadly to conditions for nucleic acid hybridization and washing. Guidance for performing hybridization reactions can be found in Ausubel, et al. (2002) “Short Protocols in Molecular Biology”, (5^{th} Ed.) John Wiley & Sons, NY. Exemplary specific hybridization conditions include but are not limited to: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and (4) very high stringency hybridization conditions are 0.5M sodium phosphate, 7% SDS at 65° C., followed by one or more washes at 0.2×SSC, 1% SDS at 65° C.

The term “low level” or “low” as used in relation to a marker such as CD127 is well known in the art and refers to the expression level of the cell marker of interest (e.g., CD 127), in that the expression level of the cell marker is low by comparison with the expression level of that cell marker in other cells in a population of cells being analyzed as a whole. More particularly, the term “low” refers to a distinct population of cells that express the cell marker at a lower level than one or more other distinct population of cells. Accordingly CD127^{low} refers to cells of a type that stains slightly or dully when contacted with a labeled CD127 antibody, e.g., at a level that is higher than a CD127− subpopulation but lower than the CD127+ subpopulation.

“Mammal,” as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to alpacas, armadillos, capybaras, cats, camels, chimpanzees, chinchillas, cattle, dogs, goats, gorillas, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington DC.

“Myeloid-derived suppressor cells” or “MDSCs” are a heterogeneous group of immune cells of myeloid lineage (a family of cells that originate from bone marrow stem cells). MDSCs strongly expand in pathological situations such as chronic infections and cancer, as a result of altered hematopoiesis. MDSCs are discriminated from other myeloid cell types in which they possess strong immunosuppressive activities rather than immunostimulatory properties. Similar to other myeloid cells, MDSCs interact with other immune cell types including T cells, dendritic cells, macrophages and natural killer cells to regulate their functions. Clinical evidence has shown that cancer tissues with high infiltration of MDSCs are associated with poor patient prognosis and resistance to therapies (Mantovani A., December 2010, “The growing diversity and spectrum of action of myeloid-derived suppressor cells”, European Journal of Immunology. 40 (12): 3317-20. doi:10.1002/eji.201041170. PMID 21110315; Allavena P, Mantovani A., February 2012, “Immunology in the clinic review series; focus on cancer: tumour-associated macrophages: undisputed stars of the inflammatory tumour microenvironment”, Clinical and Experimental Immunology. 167 (2): 195-205. doi:10.1111/j.1365-2249.2011.04515.x, PMC 3278685. PMID 22235995; Galdiero M R et al., (November 2013). “Tumor associated macrophages and neutrophils in cancer”, Immunobiology. 218 (11): 1402-10. doi:10.1016/j.imbio.2013.06.003. PMID 23891329; Gabrilovich D I et al., “Coordinated regulation of myeloid cells by tumors”, Nature Reviews. Immunology. 12 (4): 253-68. doi:10.1038/nri3175. PMC 3587148. PMID 22437938.

MDSCs consist of two large groups of cells: granulocytic or polymorphonuclear (PMN-MDSCs or gMDSCs) and monocytic (M-MDSC). PMN-MDSC or gMDSC are phenotypically and morphologically similar to neutrophils, whereas M-MDSC are more similar to monocytes (Gabrilovich D I et al., “Coordinated regulation of myeloid cells by tumors”, Nat Rev Immunol. 2012; 12(4):253-268). Also the existence of a third small population of MDSCs that are represented by cells with colony forming activity and other myeloid precursors has been reported which are referred to as early-stage MDSC (eMDSC) (Dumitru C A, et al., “Neutrophils and granulocytic myeloid-derived suppressor cells: immunophenotyping, cell biology and clinical relevance in human oncology”, Cancer Immunol Immunother. 2012; 61(8):1155-11673).

“Nucleic acid” or “nucleic acid sequence,” as used herein, refers broadly to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones. Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide.

“Operatively linked”, as used herein, refers broadly to when two DNA fragments are joined such that the amino acid sequences encoded by the two DNA fragments remain in-frame.

“Paratope,” as used herein, refers broadly to the part of an antibody which recognizes an antigen (e.g., the antigen-binding site of an antibody). Paratopes may be a small region (e.g., 15-22 amino acids) of the antibody's Fv region and may contain parts of the antibody's heavy and light chains. See Goldsby, et al. Antigens (Chapter 3) Immunology (5^{th} Ed.) New York: W.H. Freeman and Company, pages 57-75.

“Patient,” as used herein, refers broadly to any animal who is in need of treatment either to alleviate a disease state or to prevent the occurrence or reoccurrence of a disease state. Also, “patient” as used herein, refers broadly to any animal who has risk factors, a history of disease, susceptibility, symptoms, signs, was previously diagnosed, is at risk for, or is a member of a patient population for a disease. The patient may be a clinical patient such as a human or a veterinary patient such as a companion, domesticated, livestock, exotic, or zoo animal. The term “subject” may be used interchangeably with the term “patient”. In preferred embodiments of the inventions disclosed herein, the patient is a human.

“Polypeptide,” “peptide” and “protein,” are used interchangeably and refer broadly to a polymer of amino acid residues. The terms apply to amino acid polymers in which one or more amino acid residue is an analog or mimetic of a corresponding naturally occurring amino acid, as well as to naturally occurring amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer. Polypeptides can be modified, e.g., by the addition of carbohydrate residues to form glycoproteins. The terms “polypeptide,” “peptide” and “protein” include glycoproteins, as well as non-glycoproteins.

“Promoter,” as used herein, refers broadly to an array of nucleic acid sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation.

“Prophylactically effective amount,” as used herein, refers broadly to the amount of a compound that, when administered to a patient for prophylaxis of a disease or prevention of the reoccurrence of a disease, is sufficient to effect such prophylaxis for the disease or reoccurrence. The prophylactically effective amount may be an amount effective to prevent the incidence of signs and/or symptoms. The “prophylactically effective amount” may vary depending on the disease and its severity and the age, weight, medical history, predisposition to conditions, preexisting conditions, of the patient to be treated.

“Prophylaxis,” as used herein, refers broadly to a course of therapy where signs and/or symptoms are not present in the patient, are in remission, or were previously present in a patient. Prophylaxis includes preventing disease occurring subsequent to treatment of a disease in a patient. Further, prevention includes treating patients who may potentially develop the disease, especially patients who are susceptible to the disease (e.g., members of a patent population, those with risk factors, or at risk for developing the disease).

“Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

“Specifically (or selectively) binds” to an antibody or “specifically (or selectively) immunoreactive with,” or “specifically interacts or binds,” as used herein, refers broadly to a protein or peptide (or other epitope), refers, in some embodiments, to a binding reaction that is determinative of the presence of the protein in a heterogeneous population of proteins and other biologics. For example, under designated immunoassay conditions, the specified antibodies bind to a particular protein at least two times greater than the background (non-specific signal) and do not substantially bind in a significant amount to other proteins present in the sample. Typically a specific or selective reaction will be at least twice background signal or noise and more typically more than about 10 to 100 times background.

“Specifically hybridizable” and “complementary” as used herein, refer broadly to a nucleic acid can form hydrogen bond(s) with another nucleic acid sequence by either traditional Watson-Crick or other non-traditional types. The binding free energy for a nucleic acid molecule with its complementary sequence is sufficient to allow the relevant function of the nucleic acid to proceed, e.g., RNAi activity. Determination of binding free energies for nucleic acid molecules is well known in the art. See, e.g., Turner, et al. (1987) CSH Symp. Quant. Biol. LI: 123-33; Frier, et al. (1986) PNAS 83: 9373-77; Turner, et al. (1987) J. Am. Chem. Soc. 109: 3783-85. A percent complementarity indicates the percentage of contiguous residues in a nucleic acid molecule that can form hydrogen bonds (e.g., Watson-Crick base pairing) with a second nucleic acid sequence (e.g., about at least 5, 6, 7, 8, 9,10 out of 10 being about at least 50%, 60%, 70%, 80%, 90%, and 100% complementary, inclusive). “Perfectly complementary” or 100% complementarity refers broadly all of the contiguous residues of a nucleic acid sequence hydrogen bonding with the same number of contiguous residues in a second nucleic acid sequence. “Substantial complementarity” refers to polynucleotide strands exhibiting about at least 90% complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically may differ by at least 5 nucleotides.

“Signs” of disease, as used herein, refers broadly to any abnormality indicative of disease, discoverable on examination of the patient; an objective indication of disease, in contrast to a symptom, which is a subjective indication of disease.

“Solid support,” “support,” and “substrate,” as used herein, refers broadly to any material that provides a solid or semi-solid structure with which another material can be attached including but not limited to smooth supports (e.g., metal, glass, plastic, silicon, and ceramic surfaces) as well as textured and porous materials. Exemplary solid supports include beads, such as activated beads, magnetically responsive beads, or fluorescently labeled beads.

“Subjects” as used herein, refers broadly to anyone suitable to be treated according to the presently disclosed inventions include, but are not limited to, avian and mammalian subjects, and are preferably mammalian. Mammals in the context of the presently disclosed inventions include, but are not limited to, canines, felines, bovines, caprines, equines, ovines, porcines, rodents (e.g., rats and mice), lagomorphs, primates, humans. Any mammalian subject in need of being treated according to the presently disclosed inventions is suitable. Human subjects of any gender and at any stage of development (i.e., neonate, infant, juvenile, adolescent, adult, elderly) can be treated according to the present invention. The present invention may also be carried out on animal subjects, particularly mammalian subjects such as mice, rats, dogs, cats, cattle, goats, sheep, and horses for veterinary purposes, and for drug screening and drug development purposes. “Subjects” is used interchangeably with “patients”. In preferred embodiments of the disclosed invention, the subject is a human.

“Symptoms” of disease as used herein, refers broadly to any morbid phenomenon or departure from the normal in structure, function, or sensation, experienced by the patient and indicative of disease.

“Therapy,” “therapeutic,” “treating,” or “treatment”, as used herein, refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms (e.g., tumor growth, metastasis). Therapy also encompasses “prophylaxis”. The term “reduced”, for purpose of therapy, refers broadly to the clinical significant reduction in signs and/or symptoms. Therapy includes treating relapses or recurrent signs and/or symptoms (e.g., tumor growth, metastasis). Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms (e.g., tumor growth, metastasis).

“Variable region” or “VR,” as used herein, refers broadly to the domains within each pair of light and heavy chains in an antibody that are involved directly in binding the antibody to the antigen. Each heavy chain has at one end a variable domain (V_{H}) followed by a number of constant domains. Each light chain has a variable domain (V_{L}) at one end and a constant domain at its other end; the constant domain of the light chain is aligned with the first constant domain of the heavy chain, and the light chain variable domain is aligned with the variable domain of the heavy chain.

“Vector,” as used herein, refers broadly to a plasmid, cosmid, phagemid, phage DNA, or other DNA molecule which is able to replicate autonomously in a host cell, and which is characterized by one or a small number of restriction endonuclease recognition sites at which such DNA sequences may be cut in a determinable fashion without loss of an essential biological function of the vector, and into which DNA may be inserted in order to bring about its replication and cloning. The vector may further contain a marker suitable for use in the identification of cells transformed with the vector.

The techniques and procedures are generally performed according to conventional methods well known in the art and as described in various general and more specific references that are cited and discussed throughout the present specification. See, e.g., Sambrook, et al. (2001) Molec. Cloning: Lab. Manual [3^{rd} Ed] Cold Spring Harbor Laboratory Press. Standard techniques may be used for recombinant DNA, oligonucleotide synthesis, and tissue culture, and transformation (e.g., electroporation, lipofection). Enzymatic reactions and purification techniques may be performed according to manufacturer's specifications or as commonly accomplished in the art or as described herein. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques may be used for chemical syntheses, chemical analyses, pharmaceutical preparation, formulation, and delivery, and treatment of patients.

### EXAMPLES

The invention now being generally described, it will be more readily understood by reference to the following examples, which are included merely for purposes of illustration of certain aspects and embodiments of the present invention, and are not intended to limit the invention.

### Example 1: NEO-201 mAb Targets and May be Used to Deplete Human Granulocytic Myeloid Derived Suppressor Cells

### Background

### Myeloid Derived Suppressor Cells

Myeloid derived suppressor cells (MDSC) are a heterogeneous population of immature myeloid cells that are increased in cancer, inflammation, and infection. Myeloid derived suppressor cells are associated with cancer evasion as well as tumor progression and metastasis by suppressing the antitumor immune response. The heterogeneous population of immature myeloid cells include monocytic-MDSCs (mMDSCs) and granulocytic-MDSCs (gMDSC) (Zilio S. and Serafini P., “Neutrophils and granulocytic MDSC: The Janus God of cancer immunotherapy”, Vaccine 2016; 4 (3):31; Aarts CEM and Kuijpers T W., “Neutrophils as myeloid-derived suppressor cells”, Eur J Clin Invest 2018; November,48 Suppl2:e12989.) Human MDSC express myeloid cell markers such as CD11b positive and CD33 positive, but usually are negative for HLA-DR, CD3, CD19 and CD57. Monocytic MDSCs are usually have HLA-DR negative, CD11b positive, CD33 positive and CD14 positive phenotype. Granulocytic MDSC are usually characterized by HLA-DR negative, CD11b positive, CD33 positive, CD15 positive phenotype (Zilio S. and Serafini P., “Neutrophils and granulocytic MDSC: The Janus God of cancer immunotherapy”, Vaccine 2016; 4 (3):31).

As discussed previously hereon, clinical studies have demonstrated the prognostic role of tumor infiltrating neutrophils, elevated blood neutrophils and elevated blood neutrophil/lymphocyte ratio and particularly that such cells are associated with poor clinical outcome in different human cancers. These results highlight the importance and relevance of neutrophils in cancer biology.

Neutrophils are primary inflammatory cells and essential to protect the host against invading pathogens such as bacteria and fungi. Recently, neutrophils have been shown high functional plasticity and can adopt protumor and antitumor activity. Protumor neutrophils function as repressors of adaptive immune responses in cancer. An expansion of immature and mature neutrophils has been observed to suppress T-cell proliferation. Protumor neutrophils are functionally related to the gMDSCs. Myeloid derived suppressor cells play an important part in suppression of host immune responses through several mechanisms such as production of (a) arginase 1, (b) release of reactive oxygen species (ROS), (c) release of nitric oxide and (d) secretion of suppressive cytokines (Donskov F. et al., “Immunomonitoring and prognostic relevance of neutrophils in clinical trials”, Seminars in Cancer Biology 2013; 23: 200-2071; Sagiv J et al., “Phenotypic diversity and plasticity in circulating neutrophil subpopulations in cancer”, Cell Reports 2015; 10:562-573.)

Granulocytes are derived from hematopoietic stem cells in the bone marrow which is controlled by granulocyte colony-stimulating factor (G-CSF). Under pathological conditions, MDSCs can be generated in the bone marrow in responses to the cancer and infection elicited factors such as G-CSF, GM-CSF, IL-6, IL-1-beta, prostaglandin E2 (PGE2), TNF-alpha and VEGF (Lechner M G, et al., “Characterization of cytokine-induced myeloid-derived suppressor cells from normal peripheral blood mononuclear cells”, J Immunol 2010; 185:2273-2284).

### NEO-201 Monoclonal Antibody

NEO-201 is a therapeutic IgG1 humanized mAb reactive against many different carcinomas, but not reactive against most normal epithelial tissues. No reactivity was observed with NEO-201 in subsets of hematopoietic cells except for CD15+ granulocytes and circulating Treg cells. Functional analysis revealed that NEO-201 can engage in ADCC and CDC to kill tumor cells. Previous studies showed that NEO-201 attenuates growth of human tumor xenografts in mice and demonstrates safety/tolerability in non-human primates with a transient decrease in neutrophils being the only adverse effect observed. A first in human clinical trial evaluating NEO-201 in adults with chemo-resistant solid tumors is ongoing at the NIH clinical Center (Fantini M et al., “Preclinical characterization of a novel monoclonal antibody NEO-201 for the treatment of human carcinoma”, Front Immunol 2018; 8:1899; Zeligs K P et al., “Evaluation of the anti-tumor activity of the humanized monoclonal antibody NEO-201 in preclinical models of ovarian cancer”, Front Oncol. 2020; 10:805).

NEO-201 recognizes tumor-associated variants of CEACAM5 and 6 carrying core-1 and/or extended core-1 O-glycans. CEACAM1 is a potent inhibitor of natural killer (NK) cell function; binding between CEACAM1 on NK cells and CEACAM1 or CEACAM5 on tumor cells inhibits activation signaling by NKG2D, which prevents NK cell cytolysis and permits tumor cells to evade NK killing (Fantini M, et al., “The monoclonal antibody NEO-201 enhances natural killer cell cytotoxicity against tumor cells through blockade of the inhibitory CEACAM5/CEACAM1 immune checkpoint pathway”, Cancer Biotherapy and Radiopharm 2020; 35(3):190-198).

### Materials and Methods

In Vitro Generation of Human gMDSCs

An EasySepi™ direct human neutrophil isolation kit (STEMCELL, Catalog #19257) was used for immunomagnetic isolation of neutrophils directly from whole blood from healthy donors, according to the manufacturer's protocol.

Isolated neutrophils were cultured in complete RPMI1640 medium at a concentration of 5×10⁵ cells/ml. Medium was supplemented with human IL-6 (10 ng/mL, PeproTech, Inc.) and GM-CSF (10 ng/mL, PeproTech, Inc.) for 7 days at 37° C. The medium and cytokines were refreshed every 2-3 days. 7-days cultured cells were collected from flow cytometry analysis of cell phenotype.

### Phenotypic Analysis by Flow Cytometry

The phenotype of the in vitro generated gMDSCs was evaluated for the expression of HLA-DR, CD33, CD66b, CD14, CD15 and NEO-201 target antigen by flow cytometry substantially according to Lechner et al. (Lechner M G et al., “Characterization of cytokine-induced myeloid-derived suppressor cells from normal peripheral blood mononuclear cells”, J Immunol 2010; 185:2273-2284). gMDSCs were first incubated with 1 μL per test of LIVE/DEAD Fixable Aqua (Thermo Fisher Scientific, Waltham, MA, USA) in 1 mL of 1× phosphate buffered saline (PBS) (VWR International, Radnor, PA, USA) for 30 min at 4° C. to accomplish live versus dead cell discrimination. Then, cells were washed with 1×PBS and incubated with 2-5 μL of Human TruStain FcX™ (BioLegend, San Diego, CA, USA) in 100 μL of 1×PBS at room temperature for 5-10 minutes. To detect surface markers, cells were then stained in 100 μL of 1×PBS+1% BSA (Teknova, Hollister, CA, USA) for 30 min at 4° C. with 2-4 L/sample of the following anti-human mAbs: HLA-DR-PE, CD33-APC, CD14-PerCP-Cy5.5, CD15-FITC, CD66b-PE-Cy7, NEO-201-Pacific Blue (BioLegend, San Diego, CA, USA). After staining, cells were washed twice with cold 1×PBS and examined using a FACSVerse flow cytometer (BD Biosciences, San Jose, CA, USA). Analysis of cellular fluorescence was performed using BD FACSuite software (BD Biosciences, San Jose, CA, USA). Positivity was determined by using fluorescence-minus-one controls.

### ADCC Assay

Flow cytometry was used for the analysis of ADCC activity against gMDSC mediated by NEO-201. The ADCC assay was conducted substantially according to Lechner et al. (Lechner M G et al., “Characterization of cytokine-induced myeloid-derived suppressor cells from normal peripheral blood mononuclear cells”, J Immunol 2010; 185:2273-2284).

For the ADCC assay, gMDSCs generated from neutrophils from 2 healthy donors were used as target cells. After 7 days of culture in complete RPM11640 medium supplemented with human IL-6 and GM-CSF, generated gMDSC were harvested and centrifuged at 1500 rpm for 5 minutes. Supernatant was then discarded, and pellet was washed with 1×PBS. Cells were then incubated with 1 μL per test of LIVE/DEAD Fixable Aqua in 1 mL of 1×PBS for 30 min at 4° C. to accomplish live versus dead cell discrimination. Then, cells were washed with 1×PBS and stained in 100 μL of 1×PBS+1% BSA for 30 min at 4° C. with 2-44L/sample of the following anti-human mAbs: HLA-DR-PE, CD33-APC. After staining, cells were washed twice with cold 1×PBS.

On the day of the ADCC assay, PMBCs from a different healthy donor were thawed and cultured in RPMI complete medium. PBMCs were used as effector cells and added to the tubes containing gMDSCs stained with both HLA-DR-PE and CD33-APC antibodies with or without NEO-201 (10 μg/mL) at effector:target (E:T) ratios of 100:1 and 50:1. In these experiments gMDSCs treated with medium alone were used as control.

Cells were then incubated at 37° C. for 4 h. After incubation, cells were washed twice with cold 1×PBS and examined using a FACSVerse flow cytometer (BD Biosciences, San Jose, CA, USA). Analysis of cellular fluorescence was performed using BD FACSuite software (BD Biosciences, San Jose, CA, USA). Positivity was determined by using fluorescence-minus-one controls. To assess the ADCC activity mediated by NEO-201, the percentage of CD33^{pos}/HLA-DR^{neg} viable cells in gMDSCs incubated with medium alone was compared to the percentage of CD33^{pos}/HLA-DR^{neg} viable cells incubated with PBMCs alone and with PBMCs plus NEO-201.

### Results

Phenotypic Analysis of gMDSCs Generated from Human Neutrophils

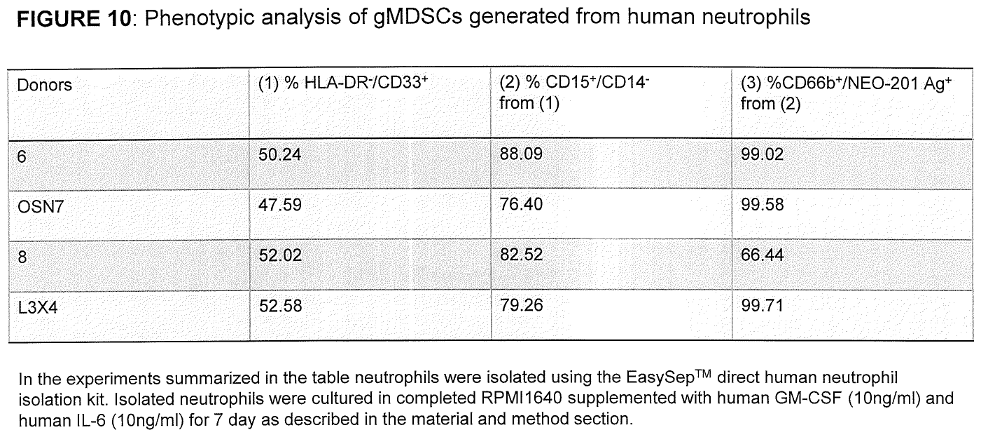

Whole blood from 4 normal donors were used in this investigation. As shown in the table in FIG. 10, 47.59 to 52.58% neutrophils treated with 10 ng/ml of human GM-CSF and 10 ng/ml of human IL-6 were HLA-DR negative and CD33 positive. 76.4% to 88.09% of the HLA-DR negative and CD33 positive population were CD15 positive and CD14 negative. 66.44% to 99.71% of the HLA-DR negative/CD33 positive/CD15 positive/CD14negative population were CD66 positive and NEO-201 positive.

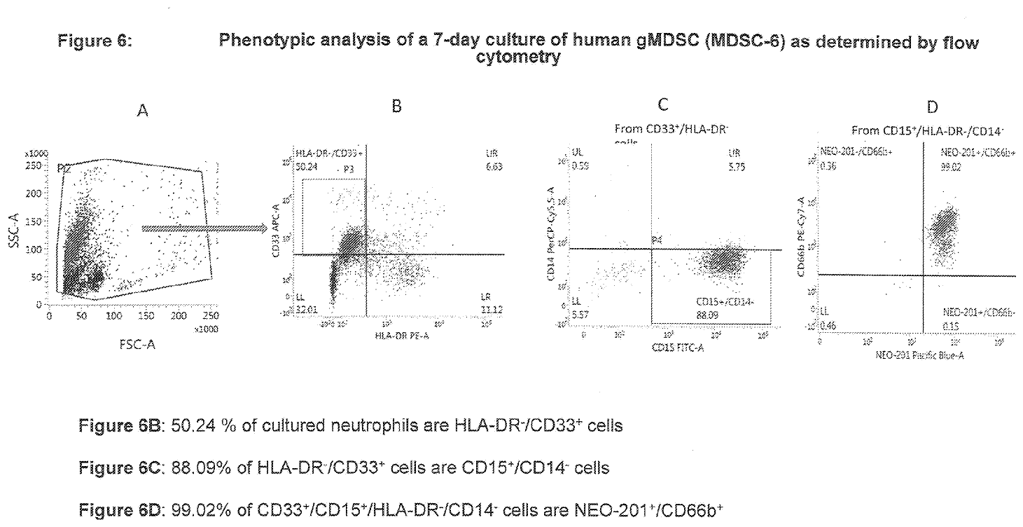

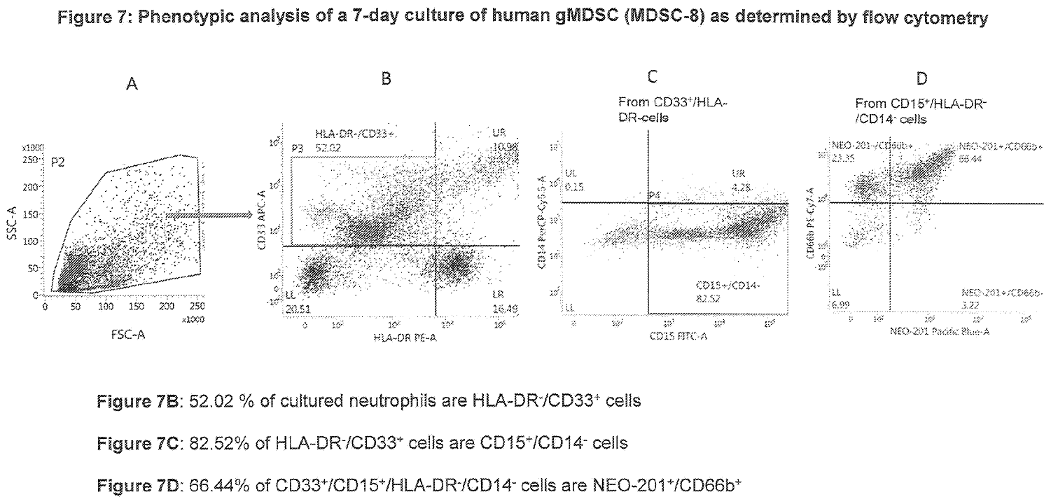

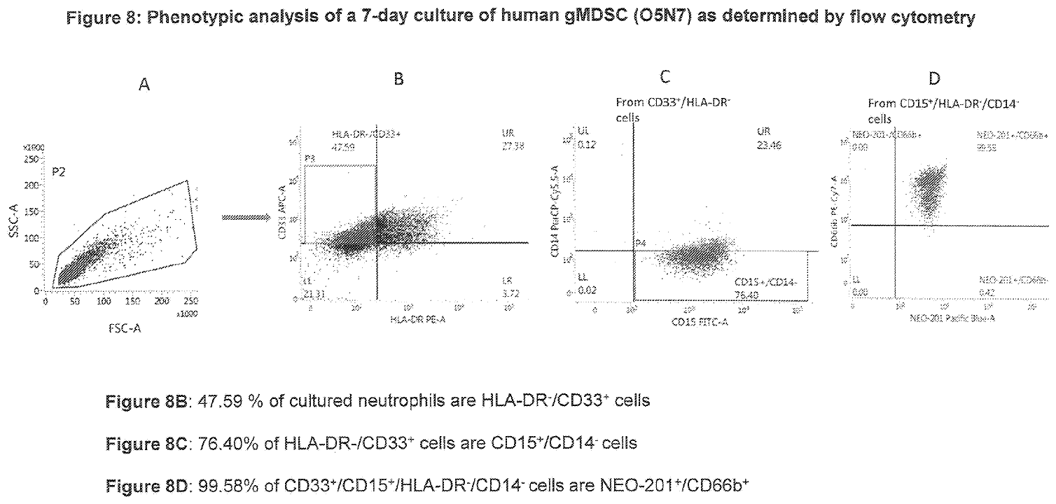

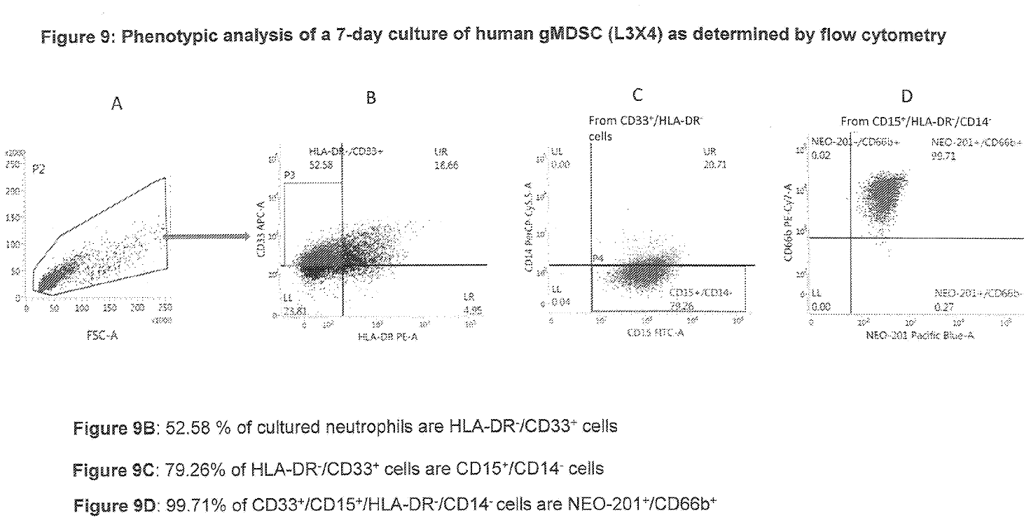

FIGS. 6-9 contain the flow cytometry analysis results for gMDSCs generated from GM-CSF and IL-6 treated neutrophils from 4 normal donors.

### ADCC Assay Results

To evaluate if NEO-201 is able to eliminate human gMDSCs through ADCC, gMDSCs generated from neutrophils from 2 healthy donors have been used as target cells in a ADCC assay performed by flow cytometry. The ADCC activity of NEO-201 was evaluated comparing the percentage of CD33^{pos}/HLA-DR^{neg} viable cells in gMDSCs incubated with medium alone with the percentage of CD33^{pos}/HLA-DR^{neg} viable cells incubated with PBMCs alone and with PBMCs plus NEO-201.

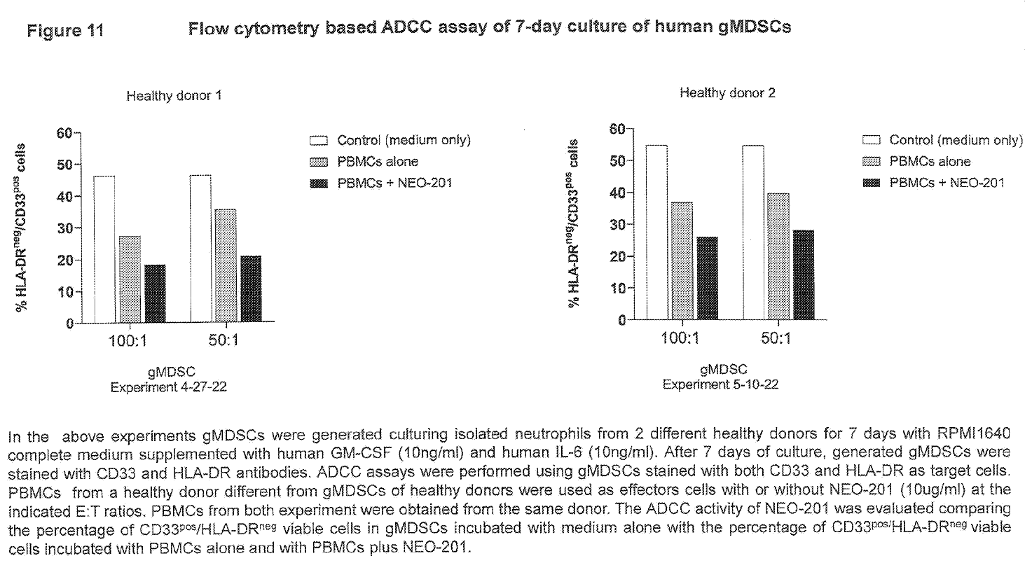

As shown in FIG. 11, when gMDSCs were incubated with PBMCs (E:T 100:1) plus NEO-201 we observed a reduction of 33.01% (18.29% vs 27.23%), and 29.5% (25.95% vs 36.83%) of CD33^{pos}/HLA-DR^{neg} viable cells compared to gMDSCs incubated with PBMCs alone (E:T 100:1) in healthy donor 1 and 2, respectively. Similar reduction of CD33^{pos}/HLA-DR^{neg} viable cells was observed comparing gMDSCs incubated with PBMCs (E:T 50:1) plus NEO-201 with gMDSCs incubated with PBMCs alone (E:T 50:1) in both healthy donors.

These data provide convincing evidence that NEO-201 is able to deplete or eliminate gMDSCs via ADCC mediated lysis in vitro. Based on these results we anticipate that NEO-201 should be useful alone and in combination with other actives, e.g., antibodies which target checkpoint inhibitors and other biologics or chemotherapeutics for alleviating immunosuppression and resistance to treatment caused by MDSCs. In particular, NEO-201 may be used to alleviate immunosuppression and resistance to treatment in individuals with cancer and chronic conditions involving MDSC-mediated immunosuppression and resistance to treatment.

### Example 2: Reduction of Percentage of Granulocytic Myeloid Derived Suppressor Cells (gMDSCs) in Peripheral Blood Mononuclear Cells (PBMCs) after Treatment with NEO-201 and Pembrolizumab

### Materials and Methods

Phenotypic Analysis of gMDSCs in PBMCs from Cancer Patients by Flow Cytometry

To investigate if NEO-201 is able to bind to and to deplete gMDSCs in cancer patients, PBMCs from 4 patients were profiled by flow cytometry for expression of specific gMDSCs markers, including HLA-DR, CD33, CD66b, CD14, CD15 and NEO-201. PBMCs were thawed and first incubated with 1 μL pertest of LIVE/DEAD Fixable Aqua (Thermo Fisher Scientific, Waltham, MA, USA) in 1 mL of 1× phosphate buffered saline (PBS) (VWR International, Radnor, PA, USA) for 30 min at 4° C. to accomplish live versus dead cell discrimination. Then, cells were washed with 1×PBS and incubated with 2-5 μL of Human TruStain FcX™ (BioLegend, San Diego, CA, USA) in 100 μL of 1×PBS at room temperature for 5-10 minutes. To gMDSCs markers, cells were then stained in 100 μL of 1×PBS+1% BSA (Teknova, Hollister, CA, USA) for 30 min at 4° C. with 2-4 μL/sample of the following anti-human mAbs: HLA-DR-PE, CD33-APC, CD14-PerCP-Cy5.5, CD15-FITC, CD66b-PE-Cy7, NEO-201-Pacific Blue (BioLegend, San Diego, CA, USA). After staining, cells were washed twice with cold 1×PBS and examined using a FACSVerse flow cytometer (BD Biosciences, San Jose, CA, USA). Analysis of cellular fluorescence was performed using BD FACSuite software (BD Biosciences, San Jose, CA, USA). Positivity was determined by using fluorescence-minus-one controls.

### Results

Phenotypic Analysis of gMDSCs from PBMCs of Cancer Patients

To evaluate if treatment with NEO-201 affected the percentage of circulating gMDSCs in cancer patients, PBMCs from 4 cancer patients enrolled in the Phase Ila clinical trial combining NEO-201 with Pembrolizumab in adults with chemo-resistant solid tumors who failed prior checkpoint inhibitor therapy (Clinical Trial NCT03476681), were used in this investigation.

Each cycle of treatment is 42 days in length consisting of 3 doses of NEO-2011V at 1.5 mg/kg every 2 weeks and one dose of Pembrolizumab 400 mg IV every 6 weeks.

Radiologic assessment, including CT, MRI, or PET-CT as appropriate, was performed prior to initial infusion and was repeated thereafter every 2 cycles (every 84 days) to correlate clinical response with modulation of percentage and function of immune cells, including gMDSCs.

The percentage of gMDSCs in PBMCs was analysed before to start the treatment with NEO-201 (C1D1 PRE), after 14 days of first infusion with NEO-201 (C1D15), before to cycle 2 (C2D1 PRE; 42 days after first infusion), and before of cycle 3 (C3D1 PRE; 84 days after first infusion) in 4 cancer patients.

gMDSC population, within alive PBMCs, was defined as HLA-DR^{neg}/CD33⁺/CD15⁺/CD14neg/CD66b⁺ cells.

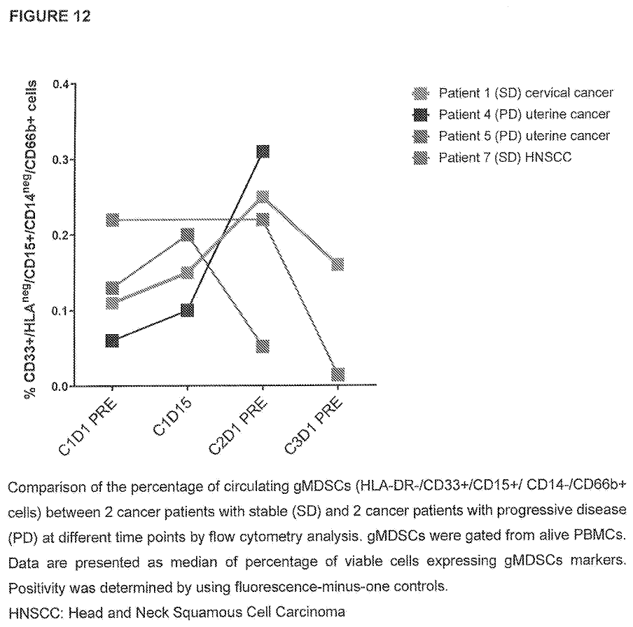

The results are contained in FIG. 12 and depict a comparison of the percentage of circulating gMDSCs (HLA-DR−/CD33+/CD15+/CD14−/CD66b+ cells) between 2 cancer patients with stable (SD) and 2 cancer patients with progressive disease (PD) at different time points by flow cytometry analysis. gMDSCs were gated from live PBMCs. Data are presented as the median of the percentage of viable cells expressing gMDSCs markers. Positivity was determined by using fluorescence-minus-one controls. In the figure “HNSCC” refers to “Head and Neck Squamous Cell Carcinoma”.

As shown in FIG. 12 one patient with Head and Neck Squamous Cell Carcinoma (HNSCC) showed a significant reduction of gMDSCs, i.e., a 93.64% reduction of gMDSCs after 84 days of treatment (C3D1 PRE) compared to baseline levels (C2D1 PRE: 0.22% vs 0.22%; C3D1 PRE: 0.014% vs 0.22%). This patient showed stable disease (SD) after treatment for more than 5 months after first infusion with NEO-201 and Pembrolizumab and he is still receiving treatment.

As further shown in FIG. 12 the other patient with SD (patient with cervical cancer) showed an increase of circulating gMDSCs at C1D15 and C2D1 PRE compared to baseline levels (C1D15: 0.15% vs 0.11%; C2D1 PRE: 0.25% vs 0.11%), but the percentage of gMDSCs started to decrease towards baseline levels at C3D1 (C3D1 PRE: 0.16% vs 0.11%). This patient showed SD after treatment for more than 8 months after first infusion with NEO-201 and Pembrolizumab, suggesting that the decrease in circulating gMDSCs started at C3D1 PRE may have continued beyond this time point and may have contributed to the stabilization of the disease.

Conversely, as further shown in FIG. 12, in one patient with uterine cancer (patient 4), the percentage of circulating gMDSCs increased after the treatment. This phenomenon correlates with the progression of the disease (PD) reported at the first re-staging (prior C3D1).

Also shown in FIG. 12, it was observed that another patient with uterine cancer (patient 5), who showed PD at the first re-staging, was found to have an initial increase at C1D15, followed by a reduction of 60% of circulating gMDSCs at C2D1 vs baseline (0.052% vs 0.13%), suggesting that the observed decrease of circulating gMDSCs, which is of significant clinical importance, may be not the only factor involved in the clinical response.

These clinical results provide convincing evidence that the administration of the NEC-201 antibody alone or in association with other treatments may be used to deplete gMDSCs in patients in need thereof, e.g., cancer patients adults with chemo-resistant solid tumors and/or who have failed prior checkpoint inhibitor therapy, and such treatment may reverse or substantially alleviate gMDSC-related tolerance or resistance to chemotherapy and/or checkpoint inhibitor therapies.

### Humanized NEO-201 Monoclonal Antibody Sequences

The sequences of the NEO-201 antibody used in these examples are as shown below:

H16C3-Abb* Heavy Chain:
(SEQ ID NO: 28)
MGWSCIIFFLVATATGVHS/QVQLVQSGAEVKKPGASVKVSCKASGYTFT
DYAMHWVRQAPGQRLEWMGLISTYSGDTKYNQNFQGRVTMTVDKSASTAY
MELSSLRSEDTAVYYCARGDYSGSRYWFAYWGQGTLVTVSS/ASTKGPSV
FPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSC
SVMHEALHNHYTQKSLSLSPGK
H16C3-Abb* Light Chain:
(SEQ ID NO: 29)
MGVPTQLLLLWLTVVVVRC/DIQMTQSPSSLSASVGDRVTITCQASENIY
GALNWYQRKPGKSPKLLIYGASNLATGMPSRFSGSGSGTDYTFTISSLQP
EDIATYYCQQVLSSPYTFGGGTKLEIKR/TVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC

The boundaries between the expression leader sequence, variable region, and constant region is delimited by a forward slash (“/”) in each sequence, and CDR sequences are shown in bold, underlined text. The antibody sequences used included the variable and constant regions shown. These include the heavy chain CDR1 of SEQ ID NO: 32, the heavy chain CDR2 of SEQ ID NO: 33, the heavy chain CDR3 of SEQ ID NO: 34, the light chain CDR1 of SEQ ID NO: 35, the light chain CDR2 of SEQ ID NO: 36, and the light chain CDR3 of SEQ ID NO: 37.

Each document cited herein is hereby incorporated by reference in its entirety.

What is claimed is:

1-102. (canceled)

103. A method of therapy, which comprises the administration of an antibody or antibody fragment which binds to glycosylated CEACAM 5 and CEACAM6 carrying core-1 and/or extended core-1 O-glycans but not to aglycosylated CEACAM 5 or aglycosylated CEACAM6; optionally NEO-201 or an antigen binding fragment thereof, which method results in one or more of the following:
(i) killing or ablating granulocyte derived myeloid derived suppressor cells (gMDSCs) in a patient in need thereof;
(ii) reversing tolerance and/or restoring innate immunity, e.g., innate antitumor immunity or innate anti-infectious immunity in a patient in need thereof by killing or ablating granulocyte myeloid derived suppressor cells (gMDSCs) in the patient;
(iii) reversing resistance or tolerance to an anti-cancer or anti-infectious agent treatment, e.g., an immune modulatory antibody, a checkpoint inhibitor antibody or fusion protein, or a chemotherapeutic agent, which resistance or tolerance involves granulocyte myeloid derived suppressor cells; or
(iv) treating or preventing cancer or infection reoccurrence by suppressing the proliferation of gMDSCs, thereby reestablishing innate immunity.

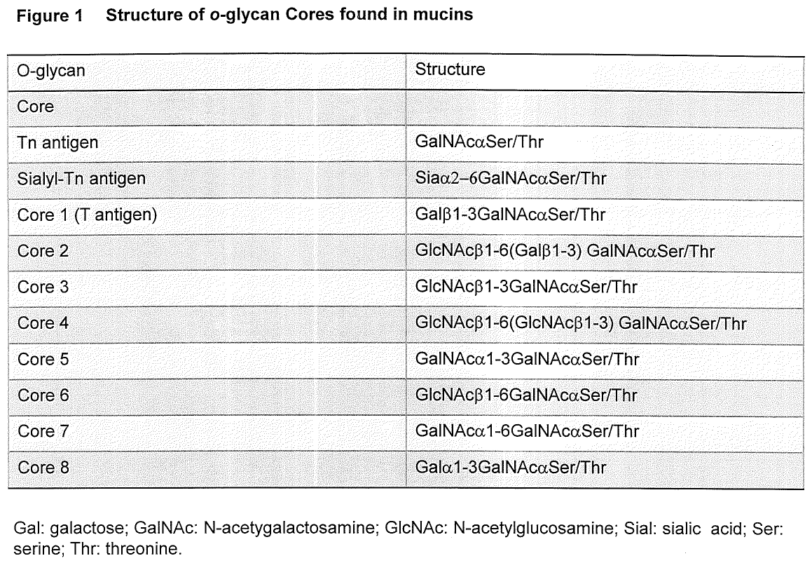

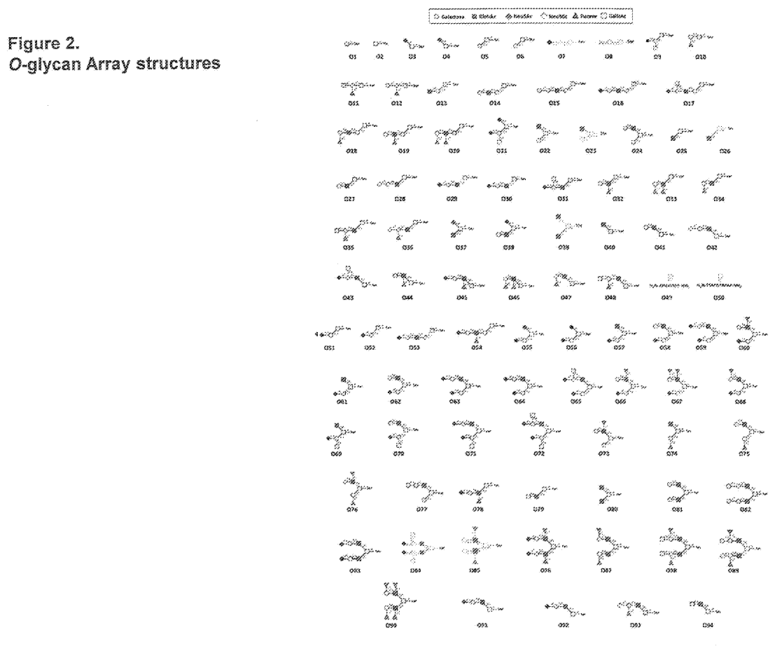

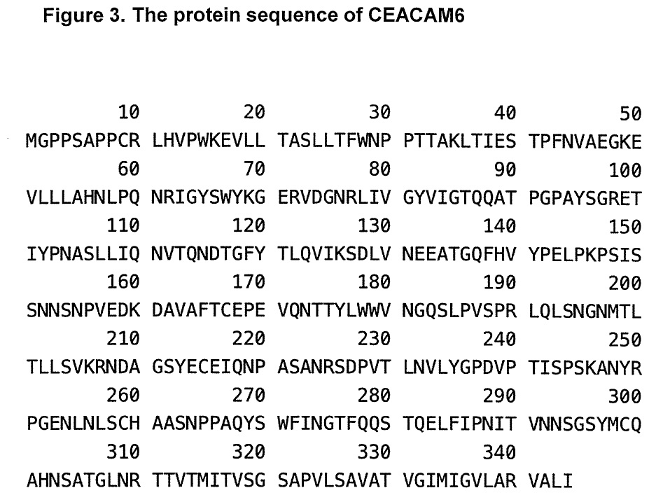

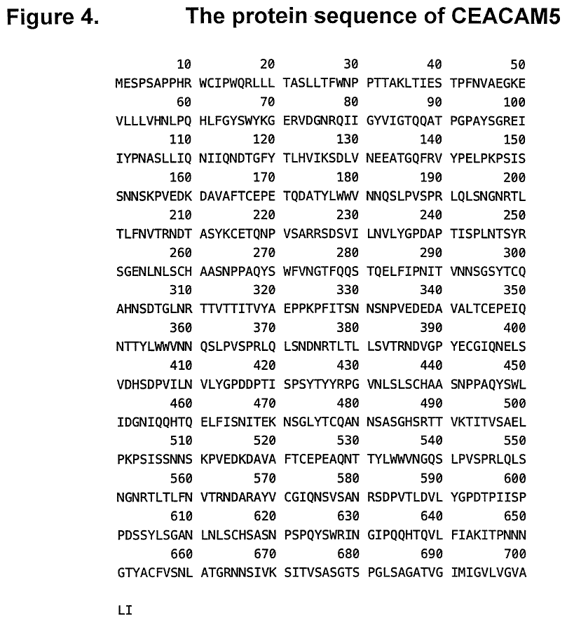

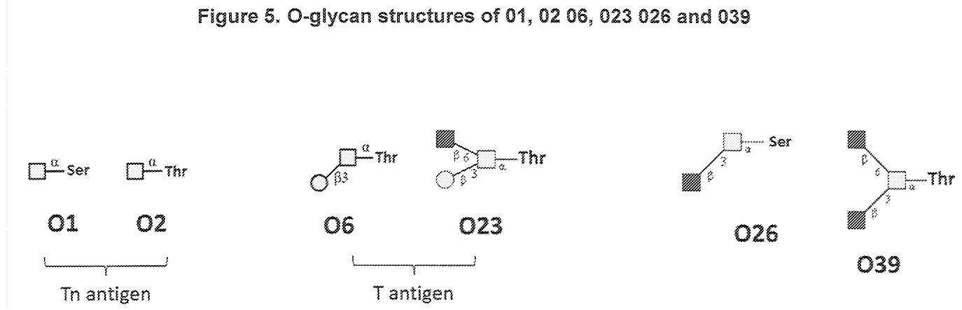

104. The method of claim 103, wherein
(i) said antibody or antibody fragment recognizes an O-glycosylated epitope binding to the threonine in the region of amino acids from 310 to 318 (RTTVTTITV) of CEACAM5 and to the Threonine and Serine in the region of amino acids 312 to 320 (TVTMITVSG) of CEACAM6;
(ii) the method of claim 103 (iii) further comprises the administration of an immune modulatory antibody, a checkpoint inhibitor antibody or fusion protein, or a chemotherapeutic agent in order to reverse such resistance or tolerance;
(iii) the method of claim 103 (iv) further comprises the administration of an immune modulatory antibody, a checkpoint inhibitor antibody or fusion protein, or a chemotherapeutic agent;
(iv) the gMDSCs express O-glycans selected from one or more of O1, O2, O6, O23, O26 and O39 O-glycans having the structure shown in the array in FIG. 2 and in FIG. 5;
(v) the gMDSCs express O6, O1 or O2 O-glycans having a structure shown in the array in FIG. 2 and/or in FIG. 5;
(vi) the gMDSCs express O6 O-glycans as shown in the array in FIG. 2 and/or in FIG. 5;
(vii) the gMDSCs express Tn antigens or Core 1, 2, 4 or 4 O-glycans having the structures shown in FIG. 1;
(viii) the patient has a cancer or infectious disease wherein the disease pathology and/or immunosuppression of innate immunity against the disease involves gMDSCs;
(ix) the antibody or antigen binding fragment is directly or indirectly linked to a cytotoxic agent, optionally a radionuclide or chemotherapeutic;
(x) the antibody or antigen binding fragment is directly or indirectly linked to a label, optionally a fluorescent or radioactive label;
(xi) the treated subject has a cancer where gMDSCs are involved in disease pathology, optionally wherein the treated cancer cells do not express or overexpress an antigen bound by NEO-201;
(xii) the treated subject has a cancer where gMDSCs are involved in disease pathology, optionally Adrenal Cancer, Anal Cancer, Bile Duct Cancer, Bladder Cancer, Bone Cancer, Brain/CNS Tumors In Adults, Brain/CNS Tumors In Children, Breast Cancer, Breast Cancer In Men, Cancer in Adolescents, Cancer in Children, Cancer in Young Adults, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Leukemia—Acute Lymphocytic (ALL) in Adults, Leukemia—Acute Myeloid (AML), Leukemia—Chronic Lymphocytic (CLL), Leukemia—Chronic Myeloid (CML), Leukemia—Chronic Myelomonocytic (CMML), Leukemia in Children, Liver Cancer, Lung Cancer, Lung Cancer—Non-Small Cell, Lung Cancer—Small Cell, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Skin Cancer—Basal and Squamous Cell, Skin Cancer—Melanoma, Skin Cancer—Merkel Cell, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor, optionally wherein the treated cancer cells do not express or overexpress an antigen bound by NEO-201;
(xiii) the treated subject has a cancer where gMDSCs are involved in disease pathology, optionally a cancer and/or a tumor selected from the group consisting of lung cancer, breast cancer, triple negative breast cancer (TNBC), colorectal cancer, liver cancer, stomach cancer, colon cancer, non-small cell lung cancer (NSCLC), bone cancer, pancreatic cancer, skin cancer, head or neck cancer, cutaneous or intraocular melanoma, uterine cancer, ovarian cancer, colorectal cancer, small intestine cancer, rectal cancer, anal cancer, fallopian tube cancer, endometrial cancer, cervical cancer, vaginal cancer, vulva cancer, Hodgkin's disease, esophageal cancer, small intestine cancer, lymph node cancer, bladder cancer, gallbladder cancer, endocrine cancer, thyroid cancer, parathyroid cancer, adrenal cancer, soft tissue sarcoma, urethra cancer, penis cancer, prostate cancer, adenocarcinoma, chronic or acute leukemia, lymphocytic lymphoma, bladder cancer, kidney or ureter cancer, renal cell carcinoma, renal pelvic carcinoma, central nervous system tumor, primary CNS tumor, spinal cord tumor, brainstem glioma, and pituitary adenoma, optionally wherein the treated cancer cells do not express or overexpress an antigen bound by NEO-201;
(xiv) the treated cancer or infection is not characterized by the expression of glycosylated CEACAM 5 and/or glycosylated CEACAM6 carrying core-1 and/or extended core-1 O-glycans; and/or is not characterized by the increased expression of glycosylated CEACAM 5 and/or glycosylated CEACAM6;
(xv) the treated subject has a cancer where gMDSCs are involved in disease pathology, and treatment elicits one or more of (i) increased T-cell response, (ii) increased antigen presentation, (iii) reduced proliferation of MDSCs and/or (iv) reduced Treg recruitment;
(xvi) the treated subject has stage I, stage II, stage Ill, or stage IV cancer involving gMDSCs;
(xvii) the antibody or fragment, optionally NEO-201, reduces, eliminates or slows or arrests the growth of tumors wherein in a patient wherein antitumor immunity was previous suppressed by gMDSCs, reduces tumor burden in the individual, inhibits tumor growth, and/or increases survival of the individual;
(xviii) the subject has an infectious condition wherein the disease pathology involves gMDSCs;
(xix) the subject has a bacterial infection involving gMDSCs, optionally Bacillus anthraces, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis and Enterococcus faecium, Escherichia coli (generally), Enterotoxigenic Escherichia coli (ETEC), Enteropathogenic E. coli, E. coli O157:H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia, Salmonella typhi, Salmonella typhimurium, Shigella sonnei, and/or Staphylococcus aureus infection;
(xx) the subject has a chronic or acute viral infection involving gMDSCs, optionally associated with Respiratory Viruses, such as, Adenoviruses, Avian influenza, Influenza virus type A, Influenza virus type B, Measles, Parainfluenza virus, Respiratory syncytial virus (RSV), Rhinoviruses, SARS-CoV, Gastro-enteric Viruses, such as, Coxsackie viruses, Enteroviruses, Poliovirus, Rotavirus, Hepatitis Viruses, such as, Hepatitis B virus, Hepatitis C virus, Bovine viral diarrhea virus (surrogate), Herpes Viruses, such as, Herpes simplex 1, Herpes simplex 2, Human cytomegalovirus, Varicella zoster virus, Retroviruses, such as, Human immunodeficiency virus 1 (HIV-1), Human immunodeficiency virus 2 (HIV-2), Simian immunodeficiency virus (SIV), Simian human immunodeficiency virus (SHIV), Viral Select Agents/Emerging Viral Pathogens, such as, Avian influenza, Dengue virus, Hantavirus, Hemorrhagic fever viruses, Lymphocytic choromeningitis virus, Smallpox virus surrogates, Cowpox, Monkeypox, Rabbitpox, Vaccinia virus, Venezuelan equine encephalomyelitis virus (VEE), West Nile virus, Yellow fever virus;
(xxi) the subject has a condition involving gMDSCs wherein gMDSCs are involved in suppressing innate immunity, optionally acquired immune deficiency syndrome (AIDS), acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, anti-synthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behcet's disease, Berger's disease, Bickerstaff's encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barre syndrome (GBS), Hashimoto's encephalopathy, Hashimoto's thyroiditis, heart disease, Henoch-Schönlein purpura, herpes gestationis (aka gestational pemphigoid), hidradenitis suppurativa, HIV infection, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenic purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosis, linear IgA disease (LAD), lupoid hepatitis (aka autoimmune hepatitis), lupus erythematosus, lymphomatoid granulomatosis, Majeed syndrome, malignancies including cancers (e.g., sarcoma, Kaposi's sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, ocular cicatricial pemphigoid, opsoclonus myoclonus syndrome, Ord's thyroiditis, palindromic rheumatism, PANDAS (pediatric autoimmune neuropsychiatric disorders associated with Streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, sarcoidosis, schizophrenia, Schmidt syndrome, Schnitzler syndrome, scleritis, scleroderma, sepsis, serum Sickness, Sjogren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome,) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis;
(xxii) the method includes detecting gMDSCs in the patient and monitoring said gMDSCs prior, during and after treatment has been completed and/or after the patient has gone into remission;
(xxiii) gMDSCs in the patient are detected prior to the treatment method to assess whether the patient will potentially benefit from NEO-201 treatment;
(xxiv) In (xxii) or (xxiii) the gMDSCs are detected in a biological sample using one or more ligands, e.g., antibodies that recognize specific biomarkers expressed on gMDSCs, optionally LOX-1, CD11b, CD15, CD66b and glycosylated CEACAM5 and CEACAM6 antigens recognized by NEO-201;
(xxv) in (xxii), (xxiii) or (xxiv) the number or concentration of gMDSCs in a sample of a subject with a cancer involving gMDSCs, wherein the patient is being treated for such cancer with NEO-201 alone or in combination with another therapeutic agent is administered to monitor the progression of the cancer (with or without treatment);
(xxvi) in (xxii), (xxiii), (xxiv) or (xxv) the number or concentration of gMDSCs in a sample of a subject with a cancer involving gMDSCs, wherein the patient is being treated for such cancer with NEO-201 alone or in combination with another therapeutic agent, is administered to determine whether NEO-201 may be beneficial in treating the cancer, alone or in combination with another therapeutic agent;
(xxvii) in (xxii), (xxiii), (xxiv), (xxv) or (xxvi) the number or concentration of gMDSCs in a sample of a subject with a cancer involving gMDSCs, is used to develop a dosing regimen of NEO-201 alone or in combination with another therapeutic agent;
(xxviii) in (xxii), (xxiii), (xxiv), (xxv), (xxvi) or (xxvii) the level of gMDSCs in a patient sample, such as a blood or biopsy sample, is used to determine cancer prognosis prior, during or after NEO-201 treatment, which method optionally comprises contacting said gMDSCs with a NEO-201 antibody;
(xxix) in (xxii), (xxiii), (xxiv), (xxv), (xxvi), (xxvii) or (xxviii) said detecting comprises cell sorting, optionally fluorescence activated cell sorting, thereby producing a sample enriched for and/or depleted of cells positive for NEO-201 antigen expression, e.g., gMDSCs;
(xxx) in (xxii), (xxiii), (xxiv), (xxv), (xxvi), (xxvii), (xxviii) or (xxix) the therapeutic method includes detecting and/or staining gMDSCs by contacting cells with a NEO-201 antibody and detecting cells that express NEO-201 wherein optionally NEO-201 is directly or indirectly labeled;
(xxxi) in any of (xxii) to (xxx) gMDSCs are isolated by contacting a patient sample with a support comprising a NEO-201 antibody and/or using other antibodies or ligands which recognize other gMDSC biomarkers, whereby said gMDSCs are retained on said support;
(xxxii) in any of (xxii) to (xxxi) the level of gMDSCs in a patient sample, such as a blood or biopsy sample, is used to determine whether a patient has or likely to develop gMDSC-mediated immunosuppression;
(xxxii) the method includes the administration of another therapeutic agent;
(xxxiii) the method includes the administration of at least one other therapeutic agent, wherein the administration of NEO-201 or other antibody which binds to glycosylated CEACAM5 and CEACAM6 carrying core-1 and/or extended core-1 O-glycans but not to aglycosylated CEACAM5 or aglycosylated CEACAM6 with the at least one other therapeutic agent potentiates the efficacy of the at least one other therapeutic agent, optionally wherein the other therapeutic comprises another therapeutic antibody, checkpoint inhibitor, chemotherapeutic and/or comprises immune cells, optionally CAR-T or CAR-NK cells, or the other therapeutic comprises another moiety which (i) ablates gMDSCs, (ii) a moiety which promotes the differentiation of gMDSCs, (iii) a moiety which inhibits the migration of gMDSCs, (iv) an epigenetic therapy which targets gMDSCs, moiety, or (v) a chemotherapeutic which targets gMDSCs or a combination of one or more of the foregoing;
(xxxiv) the method comprises the administration of NEO-201 and another therapeutic agent, and NEO-201, based on its ability to deplete gMDSCs potentiates the efficacy of the other therapeutic by potentiating innate immunity, e.g., innate anti-tumor or anti-infectious agent responses, optionally in a subject previously resistant to treatment with the other therapeutic;
(xxxv) the method includes the administration of additional therapeutic agent(s) which include, without limitation, peptides, nucleic acid molecules, small molecule compounds, antibodies and derivatives thereof;
(xxxvi) the method includes the administration of additional therapeutic agent(s), wherein such additional therapeutic agent(s) include immune checkpoint inhibitors, optionally an anti-PD-1 antibody, an anti-PD-L1 antibody, an anti-CTLA-4 antibody, an anti-CD28 antibody, an anti-TIGIT antibody, an anti-LAGS antibody, an anti-TIM3 antibody, an anti-GITR antibody, an anti-4-1BB antibody, or an anti-OX-40 antibody and/or said additional therapeutic agent(s) include one that targets adenosine A2A receptor (AZAR), B7-H3 (also known as CD276); B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA), optionally wherein the immune checkpoint inhibitors target the PD-1 axis and/or CTLA-4;
(xxxvii) the method includes the administration of additional therapeutic agent(s) wherein such additional therapeutic agent(s) include a CSF-1/1R binding agent or inhibitor;
(xxxviii) the method includes the administration of additional therapeutic agent(s) wherein such additional therapeutic agent(s) include (a) microtubule inhibitors, topoisomerase inhibitors, platins, alkylating agents, and anti-metabolites; (b) MK-2206, ON 013105, RTA 402, BI 2536, Sorafenib, ISIS-STAT3Rx, a microtubule inhibitor, a topoisomerase inhibitor, a platin, an alkylating agent, an anti-metabolite, paclitaxel, gemcitabine, doxorubicin, vinblastine, etoposide, 5-fluorouracil, carboplatin, altretamine, aminoglutethimide, amsacrine, anastrozole, azacytidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and/or vinorelbine;
(c) 1-D-ribofuranosyl-1,2,4-triazole-3 carboxamide, 9->2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir; (d) a PD-1 inhibitor or anti-PD-1 antibody such as KEYTRUDA® (pembrolizumab), OPDIVO® (nivolumab), or LIBTAYO (cemiplimab); (e) a PD-L1 inhibitor or anti-PD-L1 antibody such as TECENTRIQ (atezolizumab), IMFINZI (durvalumab), or BAVENCIO (avelumab); or (f) a CTLA-4 inhibitor or anti-CTLA-4 antibody such as YERVOY® ipilimumab;
(xxxix) the patient has been determined to be resistant to treatment with one or more actives because of gMDSC-mediated immunosuppression prior to NEO-201 treatment;
(xl) the patient has been determined to be resistant to treatment with a therapeutic antibody, optionally one that targets a checkpoint inhibitor prior to treatment with NEO-201;
(xli) the patient has been determined to be resistant to treatment with a PD-1 or CTLA-4 antagonist, optionally an antibody or fusion protein prior to treatment with NEO-201;
(xlii) the patient has developed a resistance and/or no longer responds to treatment said other therapeutic agent, e.g., a chemotherapeutic and/or a check point inhibitor, prior to treatment with NEO-201;
(xliii) the patient after treatment with NEO-201 clinically responds to the other active, optionally another therapeutic antibody or fusion protein, further optionally one that targets a checkpoint inhibitor and/or immune cells, optionally CAR-T or CAR-NK cells;
(xliv) the patient after treatment with NEO-201 clinically responds to the other active, optionally chemotherapy and/or another therapeutic antibody or a fusion protein, further optionally a PD-1 antagonist antibody such as pembrolizumab, nivolumab, cemiplimab, atezolizumab, Atezolizumab, Dostarlimab, durvalumab, lambrolizumab, or avelumab;
(xlv) the patient after treatment with NEO-201 clinically responds to the other active, optionally another therapeutic antibody or fusion protein, which targets CTLA-4, optionally Yervoy or tremelimumab and/or immune cells, optionally CAR-T or CAR-NK cells;
(xlvi) said NEO-201 antibody comprises the VH and VL CDR sequences contained in SEQ ID NO: 28 and SEQ ID NO: 29;
(xlvii) said NEO-201 antibody comprises a variable heavy chain sequence having at least 90% identity to SEQ ID NO: 38;
(xlviii) said NEO-201 antibody comprises a variable light chain sequence having at least 90% identity to SEQ ID NO: 39;
(xlix) said NEO-201 antibody comprises a variable heavy chain sequence having at least 90% identity to SEQ ID NO: 38 and a variable light chain sequence having at least 90% identity to SEQ ID NO: 39;
(l) said NEO-201 antibody comprises a heavy chain sequence having at least 90% identity to amino acids 20-470 of SEQ ID NO: 28 and a light chain sequence having at least 90% identity to amino acids 20-233 of SEQ ID NO: 29;
(li) said NEO-201 antibody or antibody fragment comprises or consists of the heavy chain sequence of amino acids 20-470 of SEQ ID NO: 28 and the light chain sequence of amino acids 20-233 of SEQ ID NO: 29;
(lii) said NEO-201 antibody or antibody fragment comprises a human IgG1 constant domain;
(liii) said NEO-201 antibody or antibody fragment is humanized;
(liv) said NEO-201 antibody or antibody fragment is conjugated to another moiety;
(lv) said NEO-201 antibody or antibody fragment is conjugated to another cytotoxic moiety, label, radioactive moiety, or affinity tag;
(lvi) said antibody or antibody fragment, preferably a NEO-201 antibody, is comprised in a chimeric antigen receptor (CAR) which is administered to the treated subject;
(lvii) said antibody or antibody fragment is comprised in a multispecific or bispecific antibody which targets at least one other antigen, optionally another tumor antigen or an antigen expressed on an immune cell, optionally wherein said other antigen is a checkpoint inhibitor or cytokine or hormone or growth factor;
(lviii) said antibody or antibody fragment is administered as an immune cell, optionally a human T or NK cell, which immune cell expresses a CAR comprising said antibody or said antibody fragment; or
(lix) any combination of (i) to (lviii).

105. A method of treatment according to claim 103 which includes the administration of a NEO-201 antibody which results in one or more of the following: (i) killing gMDSCs in a patient, optionally wherein the patient is being treated with CAR-T or CAR-NK cells, (ii) treating or preventing or reversing gMDSC mediated immunosuppression in a patient, (iii) potentiating the efficacy of CAR-T or CAR-NK therapy by administering NEO-201 in combination therewith, wherein the CAR may target any of the antigens disclosed herein.

106. The method of claim 105, which
(i) further comprises the administration of another therapeutic agent, optionally wherein said other agent is selected from (a) microtubule inhibitors, topoisomerase inhibitors, platins, alkylating agents, and anti-metabolites; (b) MK-2206, ON 013105, RTA 402, BI 2536, Sorafenib, ISIS-STAT3Rx, a microtubule inhibitor, a topoisomerase inhibitor, a platin, an alkylating agent, an anti-metabolite, paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and/orvinorelbine; (c) 1-D-ribofuranosyl-1,2,4-triazole-3 carboxamide, 9->2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir; (d) a PD-1 inhibitor or anti-PD-1 antibody such as KEYTRUDA® (pembrolizumab), OPDIVO® (nivolumab), or LIBTAYO (cemiplimab); (e) a PD-L1 inhibitor or anti-PD-L1 antibody such as TECENTRIQ (atezolizumab), IMFINZI (durvalumab), or BAVENCIO (avelumab); or (f) a CTLA-4 inhibitor or anti-CTLA-4 antibody such as YERVOY® ipilimumab, or said other agent comprises CAR-T or CAR-NK cells; or
(ii) the method further comprises the administration of an anti-cancer vaccine or CAR-T or CAR-NK cells.

107. A method of killing gMDSCs in vitro, comprising contacting a tissue, organ or cell sample suspected of comprising gMDSCs with a NEO-201 antibody.

108. The method of claim 107, wherein
(i) the tissue, organ or cell sample is obtained from a patient with a cancer or infectious disease condition;
(ii) the tissue, organ or cell sample is a bone marrow sample from an autologous or allogeneic donor;
(iii) the method further comprises contacting said gMDSCs with complement;
(iv) said gMDSCs are killed by ADCC or CDC;
(v) the method further comprises contacting said gMDSCs with effector cells, optionally wherein said effector cells comprise natural killer cells;
(vi) said gMDSCs are killed by ADCC;
(vii) said NEO-201 antibody is coupled to a cytotoxic moiety; or
(viii) any combination of (i) to (vii).

109. A method of detecting gMDSCs, comprising detecting the expression of the NEO-201 antigen by said gMDSCs and optionally one or more other gMDSC biomarkers, optionally wherein the level of gMDSCs in a patient sample, such as a blood or biopsy sample, is used to determine cancer prognosis or a treatment regimen.

110. The method of claim 109, which comprises contacting said gMDSCs with a NEO-201 antibody, wherein optionally said NEO-201 antibody is directly or indirectly coupled to a label; optionally wherein said detecting comprises cell sorting, optionally fluorescence activated cell sorting.

111. A method of staining gMDSCs, comprising contacting cells with a NEO-201 antibody, optionally wherein said NEO-201 antibody is directly or indirectly coupled to a label.

112. A method of isolating gMDSCs, comprising isolating cells that express the NEO-201 antigen and optionally at least one other gMDSC biomarker, optionally by contacting a sample containing gMDSCs with a NEO-201 antibody, optionally wherein said NEO-201 antibody is directly or indirectly labeled, further optionally wherein said sample is or comprises blood or bone marrow or a tumor biopsy sample.

113. The method of claim 107, further comprising
(i) separating NEO-201 positive cells from NEO-201 negative cells, optionally wherein said gMDSCs are isolated by cell sorting, optionally fluorescence activated cell sorting or said gMDSCs are isolated by contacting sample with a support comprising a NEO-201 antibody, whereby said gMDSCs are retained on said support;
(ii) said NEO-201 antibody comprises at the CDR sequences contained in SEQ ID NO: 28 and SEQ ID NO: 29;
(iii) said NEO-201 antibody comprises a variable heavy chain sequence having at least 90% identity to SEQ ID NO: 38;
(iv) said NEO-201 antibody comprises a variable light chain sequence having at least 90% identity to SEQ ID NO: 39;
(v) said NEO-201 antibody comprises a variable heavy chain sequence having at least 90% identity to SEQ ID NO: 38 and a variable light chain sequence having at least 90% identity to SEQ ID NO: 39;
(vi) said NEO-201 antibody comprises a heavy chain sequence having at least 90% identity to amino acids 20-470 of SEQ ID NO: 28 and a light chain sequence having at least 90% identity to amino acids 20-233 of SEQ ID NO: 29;
(vii) said NEO-201 antibody comprises all six of the CDR sequences contained in SEQ ID NO: 28 and SEQ ID NO: 29;
(viii) said NEO-201 antibody comprises a human IgG1 constant domain, optionally a human IgG1 constant domain comprising at least one mutation which enhances or inhibits one or more effector functions, optionally FcR binding, FcRN binding, glycosylation, complement (C1_{q}) binding, phagocytosis, Antibody dependent cellular cytoxicity (ADCC), complement dependent cytotoxicity (CDC), antibody mediated neutralization, opsonization, or any combination of the foregoing;
(ix) said NEO-201 antibody is humanized;
(x) said NEO-201 antibody is conjugated to another moiety;
(xi) said NEO-201 antibody is conjugated to another cytotoxic moiety, label, radioactive moiety, or affinity tag; or
(xii) any combination of (i) to (xi).

114. The method of claim 108, further comprising
(i) separating NEO-201 positive cells from NEO-201 negative cells, optionally wherein said gMDSCs are isolated by cell sorting, optionally fluorescence activated cell sorting or said gMDSCs are isolated by contacting sample with a support comprising a NEO-201 antibody, whereby said gMDSCs are retained on said support;
(ii) said NEO-201 antibody comprises at the CDR sequences contained in SEQ ID NO: 28 and SEQ ID NO: 29;
(iii) said NEO-201 antibody comprises a variable heavy chain sequence having at least 90% identity to SEQ ID NO: 38;
(iv) said NEO-201 antibody comprises a variable light chain sequence having at least 90% identity to SEQ ID NO: 39;
(v) said NEO-201 antibody comprises a variable heavy chain sequence having at least 90% identity to SEQ ID NO: 38 and a variable light chain sequence having at least 90% identity to SEQ ID NO: 39;
(vi) said NEO-201 antibody comprises a heavy chain sequence having at least 90% identity to amino acids 20-470 of SEQ ID NO: 28 and a light chain sequence having at least 90% identity to amino acids 20-233 of SEQ ID NO: 29;
(vii) said NEO-201 antibody comprises all six of the CDR sequences contained in SEQ ID NO: 28 and SEQ ID NO: 29;
(viii) said NEO-201 antibody comprises a human IgG1 constant domain, optionally a human IgG1 constant domain comprising at least one mutation which enhances or inhibits one or more effector functions, optionally FcR binding, FcRN binding, glycosylation, complement (Clq) binding, phagocytosis, Antibody dependent cellular cytoxicity (ADCC), complement dependent cytotoxicity (CDC), antibody mediated neutralization, opsonization, or any combination of the foregoing;
(ix) said NEO-201 antibody is humanized;
(x) said NEO-201 antibody is conjugated to another moiety;
(xi) said NEO-201 antibody is conjugated to another cytotoxic moiety, label, radioactive moiety, or affinity tag; or
(xii) any combination of (i) to (xi).

115. The method of claim 109, further comprising
(i) separating NEO-201 positive cells from NEO-201 negative cells, optionally wherein said gMDSCs are isolated by cell sorting, optionally fluorescence activated cell sorting or said gMDSCs are isolated by contacting sample with a support comprising a NEO-201 antibody, whereby said gMDSCs are retained on said support;
(ii) said NEO-201 antibody comprises at the CDR sequences contained in SEQ ID NO: 28 and SEQ ID NO: 29;
(iii) said NEO-201 antibody comprises a variable heavy chain sequence having at least 90% identity to SEQ ID NO: 38;
(iv) said NEO-201 antibody comprises a variable light chain sequence having at least 90% identity to SEQ ID NO: 39;
(v) said NEO-201 antibody comprises a variable heavy chain sequence having at least 90% identity to SEQ ID NO: 38 and a variable light chain sequence having at least 90% identity to SEQ ID NO: 39;
(vi) said NEO-201 antibody comprises a heavy chain sequence having at least 90% identity to amino acids 20-470 of SEQ ID NO: 28 and a light chain sequence having at least 90% identity to amino acids 20-233 of SEQ ID NO: 29;
(vii) said NEO-201 antibody comprises all six of the CDR sequences contained in SEQ ID NO: 28 and SEQ ID NO: 29;
(viii) said NEO-201 antibody comprises a human IgG1 constant domain, optionally a human IgG1 constant domain comprising at least one mutation which enhances or inhibits one or more effector functions, optionally FcR binding, FcRN binding, glycosylation, complement (Clq) binding, phagocytosis, Antibody dependent cellular cytoxicity (ADCC), complement dependent cytotoxicity (CDC), antibody mediated neutralization, opsonization, or any combination of the foregoing;
(ix) said NEO-201 antibody is humanized;
(x) said NEO-201 antibody is conjugated to another moiety;
(xi) said NEO-201 antibody is conjugated to another cytotoxic moiety, label, radioactive moiety, or affinity tag; or
(xii) any combination of (i) to (xi).

116. The method of claim 110, further comprising
(i) separating NEO-201 positive cells from NEO-201 negative cells, optionally wherein said gMDSCs are isolated by cell sorting, optionally fluorescence activated cell sorting or said gMDSCs are isolated by contacting sample with a support comprising a NEO-201 antibody, whereby said gMDSCs are retained on said support;
(ii) said NEO-201 antibody comprises at the CDR sequences contained in SEQ ID NO: 28 and SEQ ID NO: 29;
(iii) said NEO-201 antibody comprises a variable heavy chain sequence having at least 90% identity to SEQ ID NO: 38;
(iv) said NEO-201 antibody comprises a variable light chain sequence having at least 90% identity to SEQ ID NO: 39;
(v) said NEO-201 antibody comprises a variable heavy chain sequence having at least 90% identity to SEQ ID NO: 38 and a variable light chain sequence having at least 90% identity to SEQ ID NO: 39;
(vi) said NEO-201 antibody comprises a heavy chain sequence having at least 90% identity to amino acids 20-470 of SEQ ID NO: 28 and a light chain sequence having at least 90% identity to amino acids 20-233 of SEQ ID NO: 29;
(vii) said NEO-201 antibody comprises all six of the CDR sequences contained in SEQ ID NO: 28 and SEQ ID NO: 29;
(viii) said NEO-201 antibody comprises a human IgG1 constant domain, optionally a human IgG1 constant domain comprising at least one mutation which enhances or inhibits one or more effector functions, optionally FcR binding, FcRN binding, glycosylation, complement (C1₉) binding, phagocytosis, Antibody dependent cellular cytoxicity (ADCC), complement dependent cytotoxicity (CDC), antibody mediated neutralization, opsonization, or any combination of the foregoing;
(ix) said NEO-201 antibody is humanized;
(x) said NEO-201 antibody is conjugated to another moiety;
(xi) said NEO-201 antibody is conjugated to another cytotoxic moiety, label, radioactive moiety, or affinity tag; or
(xii) any combination of (i) to (xi).

117. The method of claim 111, further comprising
(i) separating NEO-201 positive cells from NEO-201 negative cells, optionally wherein said gMDSCs are isolated by cell sorting, optionally fluorescence activated cell sorting or said gMDSCs are isolated by contacting sample with a support comprising a NEO-201 antibody, whereby said gMDSCs are retained on said support;
(ii) said NEO-201 antibody comprises at the CDR sequences contained in SEQ ID NO: 28 and SEQ ID NO: 29;
(iii) said NEO-201 antibody comprises a variable heavy chain sequence having at least 90% identity to SEQ ID NO: 38;
(iv) said NEO-201 antibody comprises a variable light chain sequence having at least 90% identity to SEQ ID NO: 39;
(v) said NEO-201 antibody comprises a variable heavy chain sequence having at least 90% identity to SEQ ID NO: 38 and a variable light chain sequence having at least 90% identity to SEQ ID NO: 39;
(vi) said NEO-201 antibody comprises a heavy chain sequence having at least 90% identity to amino acids 20-470 of SEQ ID NO: 28 and a light chain sequence having at least 90% identity to amino acids 20-233 of SEQ ID NO: 29;
(vii) said NEO-201 antibody comprises all six of the CDR sequences contained in SEQ ID NO: 28 and SEQ ID NO: 29;
(viii) said NEO-201 antibody comprises a human IgG1 constant domain, optionally a human IgG1 constant domain comprising at least one mutation which enhances or inhibits one or more effector functions, optionally FcR binding, FcRN binding, glycosylation, complement (C1_{q}) binding, phagocytosis, Antibody dependent cellular cytoxicity (ADCC), complement dependent cytotoxicity (CDC), antibody mediated neutralization, opsonization, or any combination of the foregoing;
(ix) said NEO-201 antibody is humanized;
(x) said NEO-201 antibody is conjugated to another moiety;
(xi) said NEO-201 antibody is conjugated to another cytotoxic moiety, label, radioactive moiety, or affinity tag; or
(xii) any combination of (i) to (xi).

118. The method of claim 112, further comprising
(i) separating NEO-201 positive cells from NEO-201 negative cells, optionally wherein said gMDSCs are isolated by cell sorting, optionally fluorescence activated cell sorting or said gMDSCs are isolated by contacting sample with a support comprising a NEO-201 antibody, whereby said gMDSCs are retained on said support;
(ii) said NEO-201 antibody comprises at the CDR sequences contained in SEQ ID NO: 28 and SEQ ID NO: 29;
(iii) said NEO-201 antibody comprises a variable heavy chain sequence having at least 90% identity to SEQ ID NO: 38;
(iv) said NEO-201 antibody comprises a variable light chain sequence having at least 90% identity to SEQ ID NO: 39;
(v) said NEO-201 antibody comprises a variable heavy chain sequence having at least 90% identity to SEQ ID NO: 38 and a variable light chain sequence having at least 90% identity to SEQ ID NO: 39;
(vi) said NEO-201 antibody comprises a heavy chain sequence having at least 90% identity to amino acids 20-470 of SEQ ID NO: 28 and a light chain sequence having at least 90% identity to amino acids 20-233 of SEQ ID NO: 29;
(vii) said NEO-201 antibody comprises all six of the CDR sequences contained in SEQ ID NO: 28 and SEQ ID NO: 29;
(viii) said NEO-201 antibody comprises a human IgG1 constant domain, optionally a human IgG1 constant domain comprising at least one mutation which enhances or inhibits one or more effector functions, optionally FcR binding, FcRN binding, glycosylation, complement (C1_{q}) binding, phagocytosis, Antibody dependent cellular cytoxicity (ADCC), complement dependent cytotoxicity (CDC), antibody mediated neutralization, opsonization, or any combination of the foregoing;
(ix) said NEO-201 antibody is humanized;
(x) said NEO-201 antibody is conjugated to another moiety;
(xi) said NEO-201 antibody is conjugated to another cytotoxic moiety, label, radioactive moiety, or affinity tag; or
(xii) any combination of (i) to (xi).
